(19) United States
(12) Patent Application Publication
(10) Pub. No.: US 2022/0267416 A1
(43) Pub. Date: Aug. 25, 2022

(54) HIV BINDING AGENTS

(71) Applicant: Lausanne University Hospital, Lausanne (CH)

(72) Inventors: Giuseppe Pantaleo, Lausanne (CH); Craig Fenwick, Lausanne (CH)

(73) Assignee: Lausanne University Hospital, Lausanne (CH)

(21) Appl. No.: 17/626,854

(22) PCT Filed: Jul. 15, 2020
(86) PCT No.: PCT/IB2020/056668

§ 371 Date: Jan. 13, 2022

Related U.S. Application Data
(60) Provisional application No. 62/874,042, filed on Jul. 15, 2019.
(60) Provisional application No. 62/874,057, filed on Jul. 15, 2019.

(51) Int. Cl. C07K 16/10; A61P 31/18; G01N 33/569

(52) U.S. Cl. CPC C07K 16/1063; A61P 31/18; G01N 33/56988; C07K 2317/76; C07K 2317/92; C07K 2317/515; C07K 2317/31; G01N 2333/162; C07K 2317/33; C07K 2317/51; C07K 2317/21

(57) ABSTRACT

This disclosure relates to LN02M binding agents with specificity for HIV and to methods for using the same to treat, prevent and/or ameliorate HIV infection and/or AIDS. In some embodiments, this disclosure provides a binding agent(s) comprising a variable region shown in FIGS. 6A through 6E; amino acid sequence of any mutant of FIGS. 7A through 7D and/or FIGS. 8A through 8F, and any effective (e.g., HIV neutralization) combination thereof; any one or more of SEQ ID NOS. 3-92, 95-233, 248-482, or 491-699; and/or combinations thereof.

### RELATED APPLICATIONS

This application claims priority to U.S. Ser. No. 62/874,042 filed Jul. 15, 2019 and U.S. Ser. No. 62/874,057 filed on Jul. 15, 2019, each of which being incorporated into this disclosure in their entireties.

### FIELD OF THE DISCLOSURE

This disclosure relates to binding agents with specificity for human immunodeficiency virus (HIV), methods for making the same, and to methods for using the same to treat and/or prevent HIV infection.

### BACKGROUND OF THE DISCLOSURE

As we enter the fourth decade of the HIV epidemic, significant advances have been made in the understanding of HIV pathogenesis and in the development of potent and safe antiviral drugs. More than 30 antiviral drugs have been registered and the impact of combination antiretroviral therapy (ART) on both morbidity and mortality has been remarkable. However, despite the long-term suppression of HIV replication achieved in patients with optimal adherence to ART, HIV invariably rebounds after interruption of therapy. Furthermore, successful therapy does not induce or allow restoration/development of virus-specific immune responses capable of controlling HIV replication in the absence of ART. Thus, life-long ART is needed to control HIV replication and associated disease in the large majority of HIV infected subjects.

A number of immunological interventions have been investigated in the past and currently being further developed with the goal to achieve HIV functional cure, wherein viral replication is suppressed without sustained antiviral therapy. Therapeutic vaccine strategies have been the primary intervention strategy investigated but the results have shown modest efficacy in experimental animal models and patients with the exception of a CMV-based vector HIV vaccine (50% efficacy in the NHP model). Recent studies have generated interesting results on the possibility of using anti-envelope broad neutralizing antibodies (bNabs) as therapeutic agents in HIV infection.

There is a need in the art for additional reagents for targeting HIV, especially neutralizing antibodies, and methods for using the same. This disclosure addresses those needs by providing reagents and methods that may be used to target HIV and cells and/or tissues infected by and/or harboring the same.

### BRIEF DESCRIPTION OF THE DRAWINGS

In the following a brief description of the appended figures will be given. The figures are intended to illustrate the present invention in more detail. However, they are not intended to limit the subject matter of the invention in any way.

FIG. 1 shows an overview of LN02 mutations that confer either an improved potency, modest to no effect on potency or reduced neutralization potency relative to wild type LN02 in a TZM-bl reporter assay performed with the BaL HIV-1 virus. The wild type sequence for LN02 heavy and light chain are aligned with the closely related germline sequences and the individual amino acid substitutions listed below. Substitutions that induce an increase in neutralization potency by >1.4-fold are shown in black text with a grey background, no significant change in neutralization activity are in black text alone and substitutions that induce a loss in potency against the BaL virus are in white text with a black background.

FIG. 2 shows the neutralization activity presented as IC80 values for LN02 bNab with heavy chain mutations profiled against a global panel of eight pseudo-typed HIV-1 viruses. Neutralization activity was calculated using concentration response inhibition curves for each of the antibodies and IC80 values for each of the pseudo-typed viruses is indicated by the symbol in the legend. Where the IC80 was above the maximum concentration tested, a value of 20 μg/ml was used in the graph as a point of reference for the individual bNabs.

FIG. 3 shows the neutralization activity presented as IC80 values for LN02 bNab with light chain mutations profiled against a global panel of eight pseudo-typed HIV-1 viruses. Neutralization activity was calculated using concentration response inhibition curves for each of the antibodies and IC80 values for each of the pseudo-typed viruses is indicated by the symbol in the legend. Where the IC80 was above the maximum concentration tested, a value of 20 μg/ml was used in the graph as a point of reference for the individual bNabs.

FIG. 4 shows the neutralization activity presented as IC80 values for select single and multiple mutations in LN02 bNab along with 3BNC117, 101074 and VRC01 bNabs profiled against a global panel of eight pseudo-typed viruses. Neutralization activity was calculated using concentration response inhibition curves for each of the antibodies and IC80 values for each of the pseudo-typed viruses is indicated by the symbol in the legend. Where the IC80 was above the maximum concentration tested, a value of 20 μg/ml was used in the graph as a point of reference for the individual bNabs.

FIG. 5 shows the concentration response curves for select LN02 bNab variants with percent neutralization of the indicated pseudo-typed HIV-1 viruses in a TZM-bl luciferase reporter assay. The SVA-MLV pseudo-virus used as a negative control demonstrates that the bNabs tested do not exhibit non-specific inhibition.

FIG. 6. FIG. 6A. Exemplary LN02 variable heavy chain amino acid sequences.

FIG. 6B. Additional exemplary LN02 variable heavy chain amino acid sequences. FIG. 6C. Additional exemplary LN02 variable heavy chain amino acid sequences. FIG. 6D.

Exemplary LN02 variable light chain amino acid sequences. FIG. 6E. Additional exemplary LN02 variable light chain amino acid sequences.

FIG. 7. FIG. 7A. Exemplary LN02 variable heavy chain amino acid sequences.

FIG. 7B. Additional exemplary LN02 variable heavy chain amino acid sequences. FIG. 7C. Additional exemplary LN02 variable heavy chain amino acid sequences. FIG. 7D.

Exemplary LN02 variable heavy chain amino acid sequences. FIG. 7E. Additional exemplary LN02 variable heavy chain amino acid sequences.

FIG. 8. FIG. 8A. Exemplary LN02 variable light chain amino acid sequences.

FIG. 8B. Additional exemplary LN02 variable light chain amino acid sequences. FIG. 8C. Additional exemplary LN02 variable light chain amino acid sequences. FIG. 8D. Exemplary LN02 variable light chain amino acid sequences. FIG. 8E. Additional exemplary LN02 variable light chain amino acid sequences. FIG. 8F. Additional exemplary LN02 variable light chain amino acid sequences.

FIG. 9. FIG. 9A. Neutralization of AC10 pseudovirus. FIG. 9B. Neutralization of 25710 pseudovirus. FIG. 9C. Neutralization of CH119 pseudovirus. FIG. 9D. Neutralization of TRO.11 pseudovirus. FIG. 9E. Neutralization of 246F3 pseudovirus. FIG. 9F. Neutralization of CE1176 pseudovirus. FIG. 9G. Neutralization of CN155 pseudovirus. FIG. 9H. Neutralization of BJOX pseudovirus. FIG. 9I. Neutralization of CN155 pseudovirus.

### SUMMARY OF THE DISCLOSURE

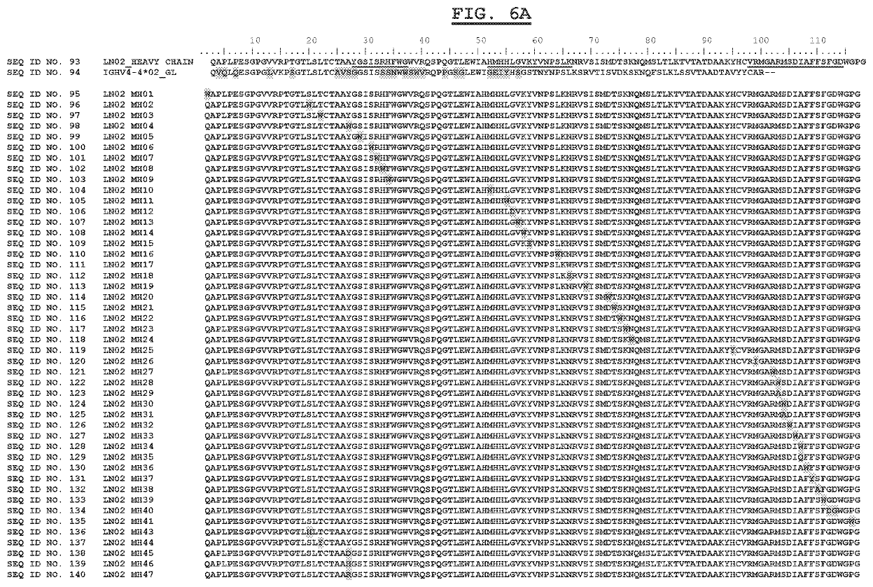

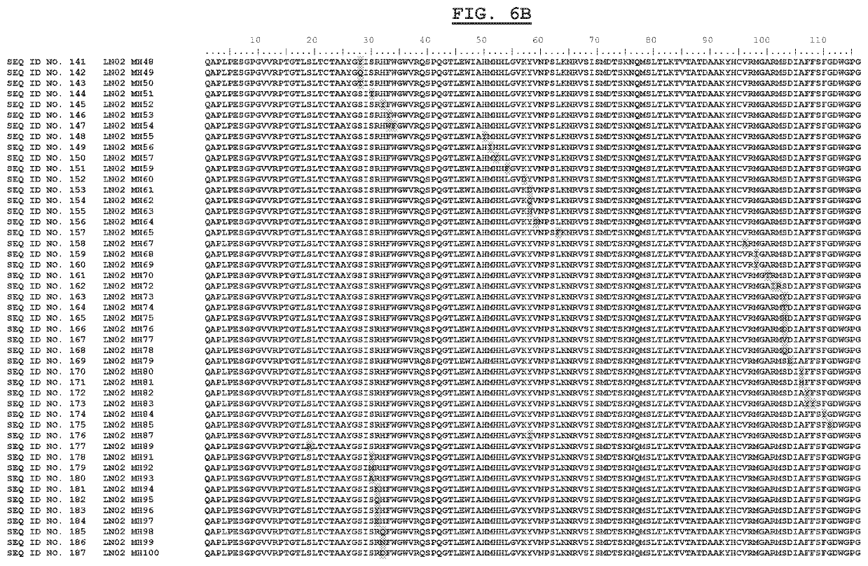

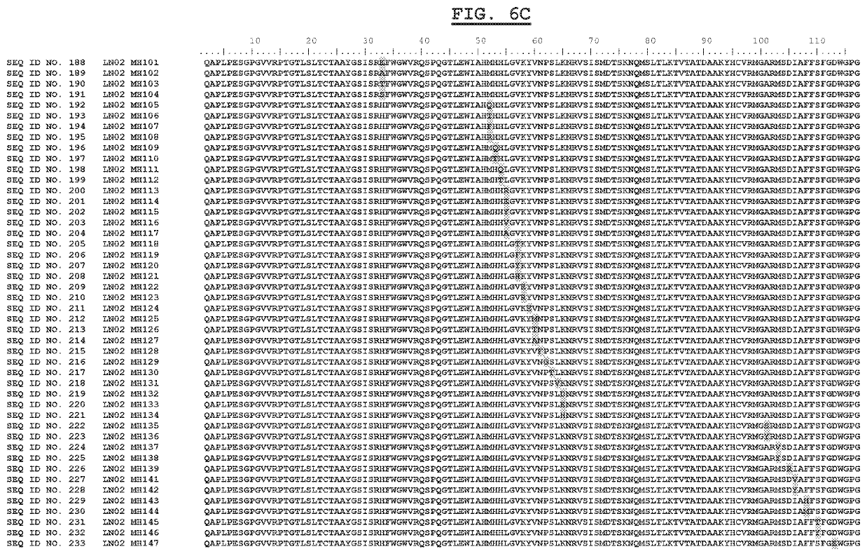

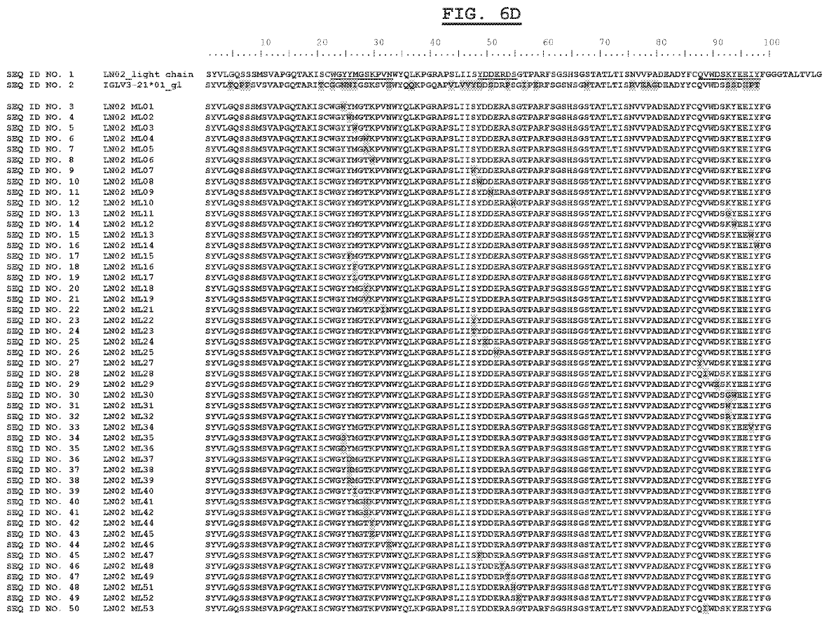

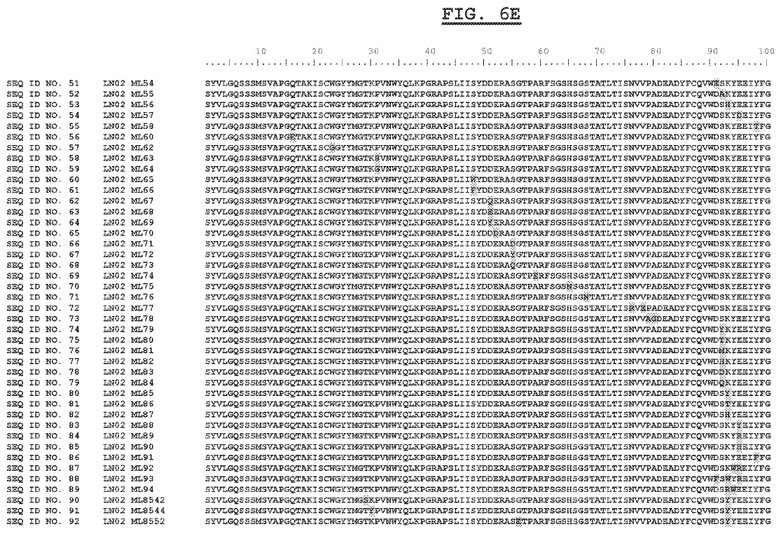

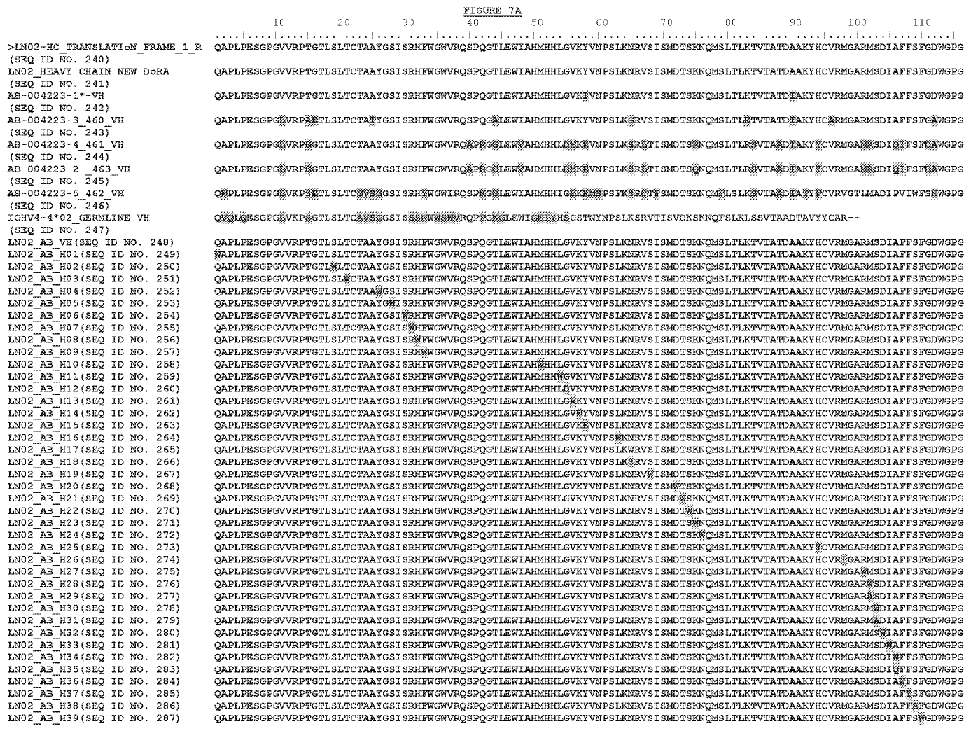

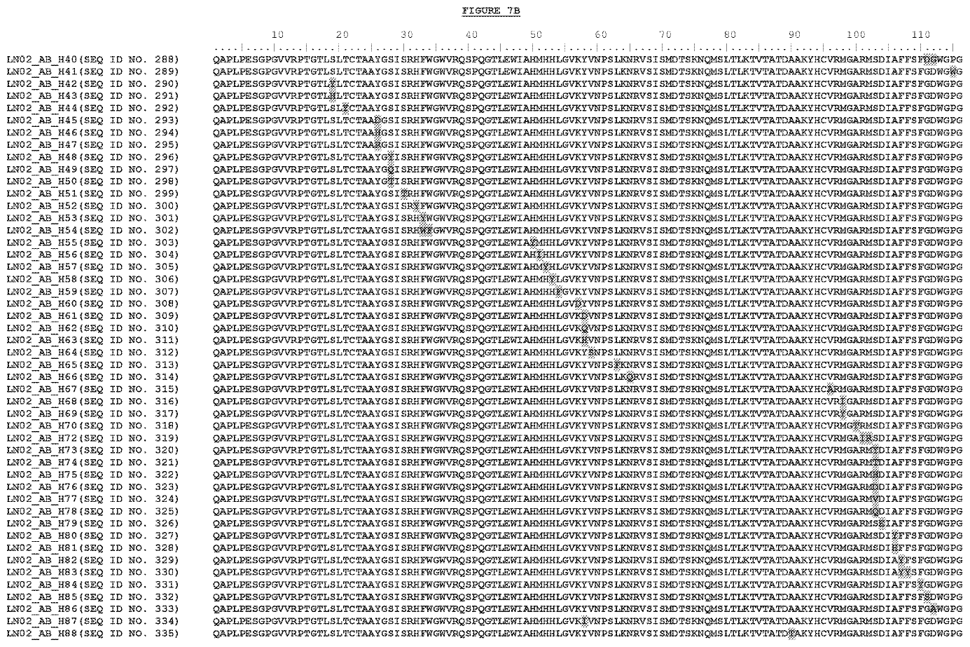

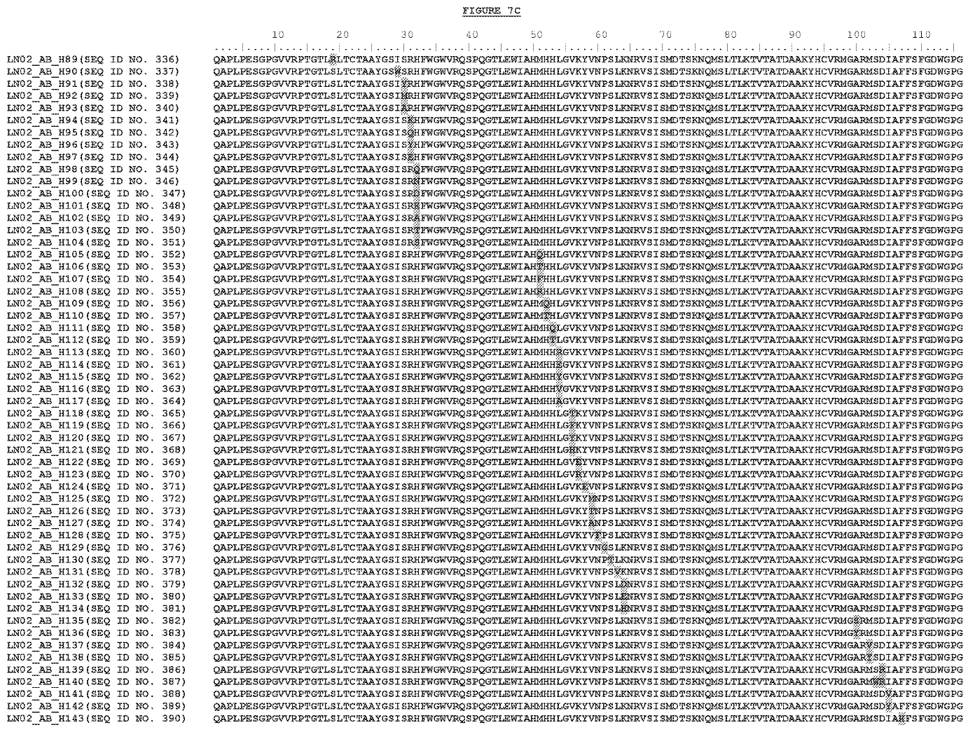

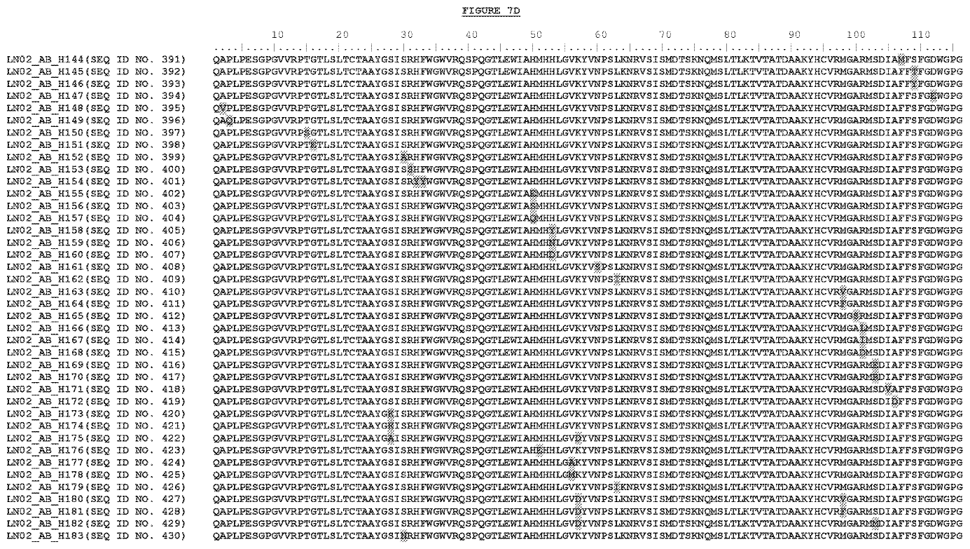

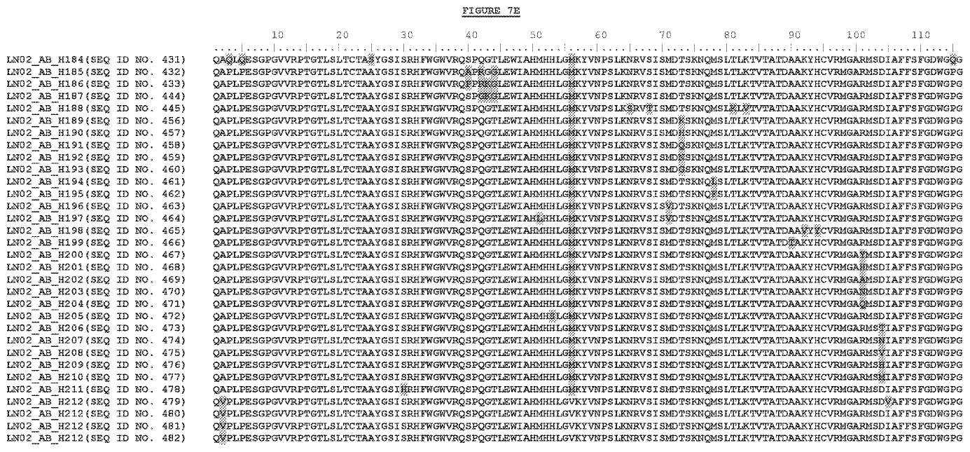

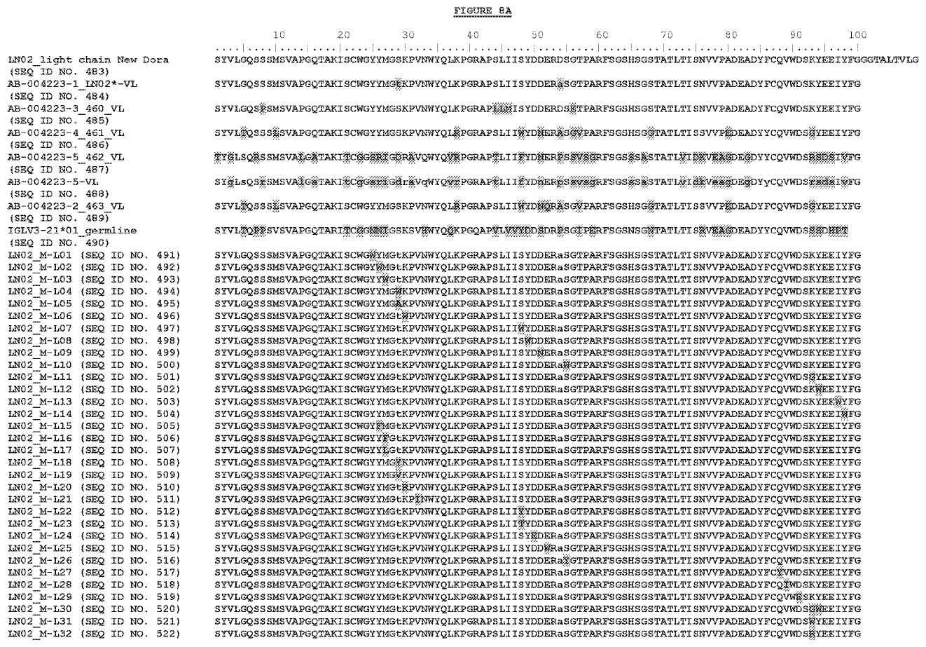

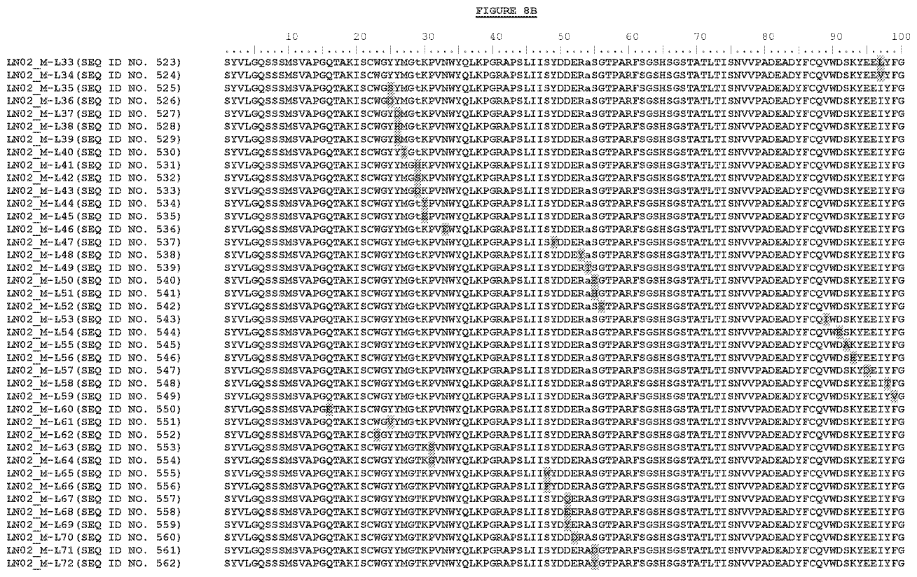

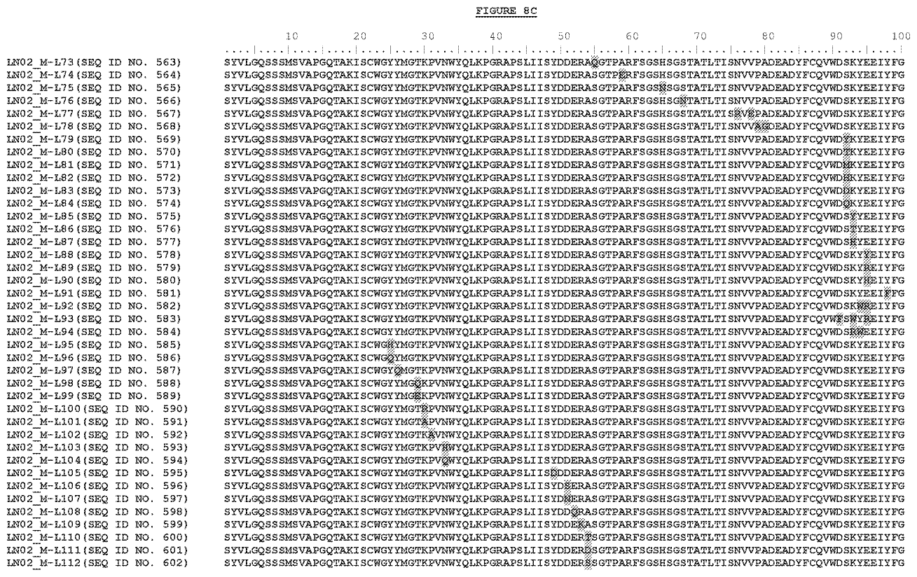

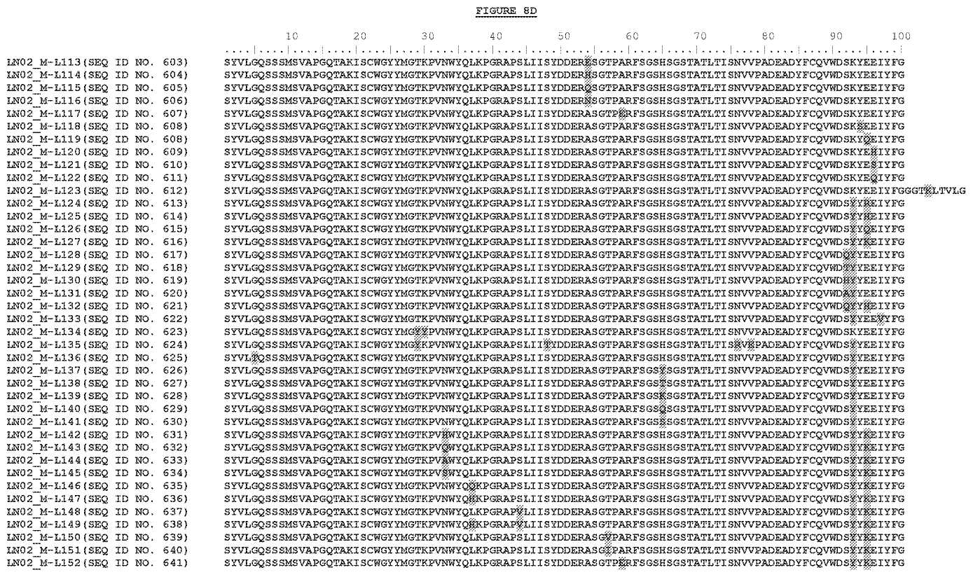

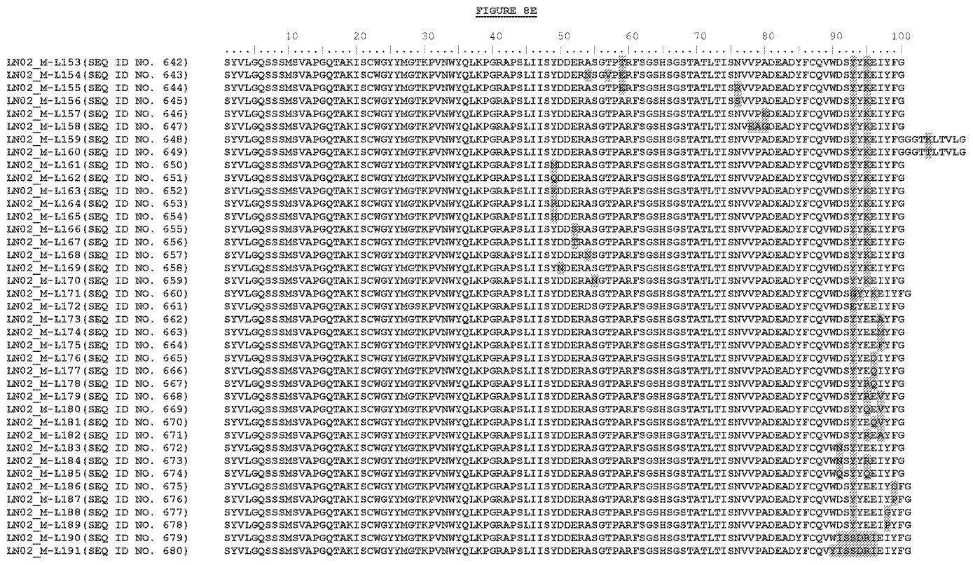

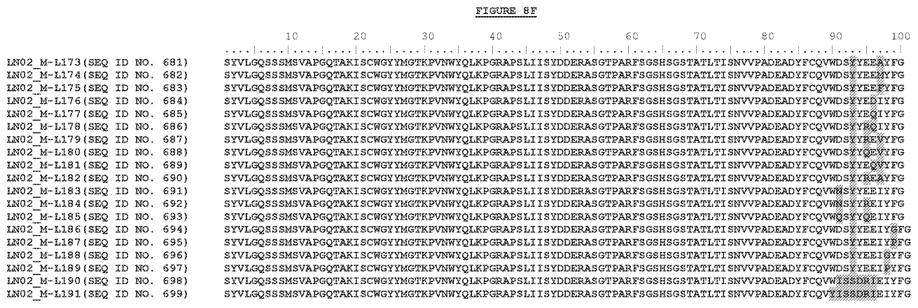

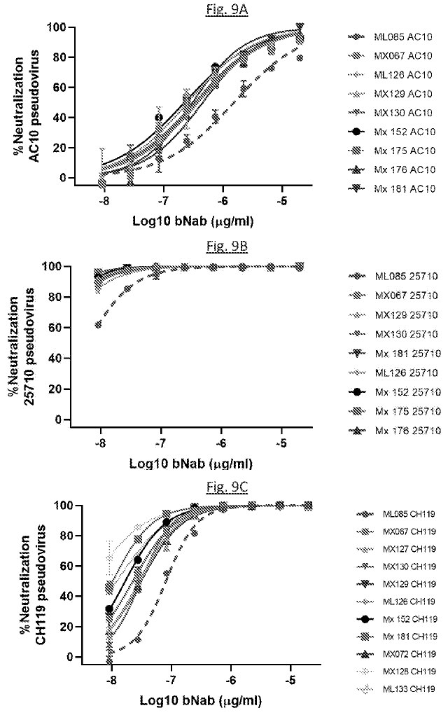

This disclosure relates to binding agents with specificity for human immunodeficiency virus (HIV), methods for producing such binding agents, as well as methods for using such binding agents to treat, prevent and/or ameliorate HIV infection. In some embodiments, this disclosure provides a binding agent(s) comprising a variable region shown in FIGS. 6A through 6E; amino acid sequence of any mutant of FIGS. 7A through 7D and/or FIGS. 8A through 8F, and any effective (e.g., HIV neutralization) combination thereof; any one or more of SEQ ID NOS. 3-92, 95-233, 248-482, or 491-699, and any effective (e.g., HIV neutralization) combination thereof; a combination of light and heavy chains shown in Table 9 (i.e., ML085, Mx152, MX067, MX129, MX130, ML126, Mx175, Mx176, and Mx181); a combination of light and heavy chains shown in Tables 10A through 10C, 11, 12A through 12D, 13A through 13D, or 14; as well as variants thereof. Reagents and methods for producing and/or using the same are also disclosed. Other embodiments are also contemplated as will be apparent to those of ordinary skill in the art from this disclosure.

### DETAILED DESCRIPTION

This disclosure relates to binding agents having binding affinity for human immunodeficiency virus (HIV). In some embodiments, the binding agent can bind HIV antigens on viral particles per se or on the surface of cells in vitro and/or in vivo. The binding agents may also bind isolated HIV antigens and/or fragments and/or derivatives thereof, typically in vitro. Also provided are methods for using such binding agents to diagnose, treat, prevent and/or ameliorate one or more diseases associated with HIV. For instance, the binding agents may be antibodies (e.g., monoclonal antibodies) that may react with and/or bind to the epitopes of HIV or polypeptides thereof. The binding agents may be useful for treating disease caused by HIV, such as Acquired Immune Deficiency Syndrome (AIDS). In some embodiments, the binding agents described herein may selectively target and/or eliminate HIV and/or HIV-infected cells containing HIV (e.g., replication competent HIV) and/or expressing proteins thereof. In some embodiments, such cells may be reservoirs for replication competent HIV. In some embodiments, binding agents having, for instance, different specificities (e.g., recognizing different epitopes) may be combined to HIV activity such as infection, replication and/or spread to other cells. In some embodiments, the binding agents described herein may also provide for the selective elimination and/or suppression of HIV or HIV-expressing cells. In some embodiments, the binding agents described herein may be used to suppress and/or eliminate HIV and/or HIV-expressing cells to treat, for instance, HIV infection and/or AIDS. Other embodiments, uses and the like are described below.

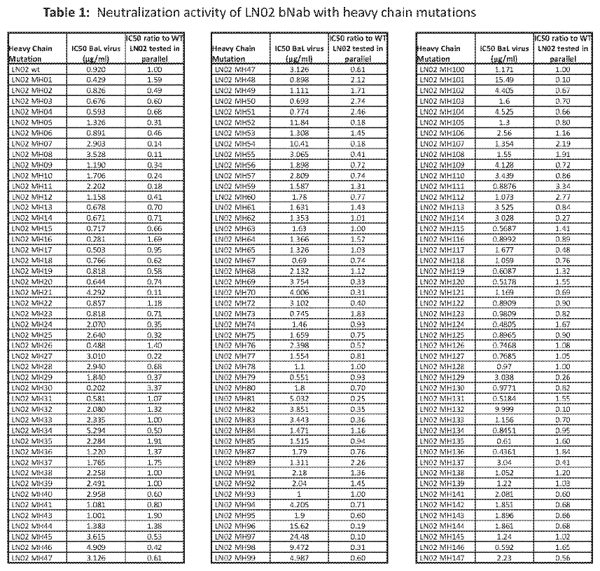

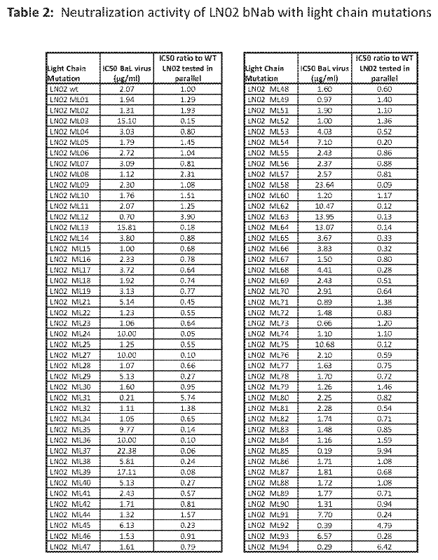

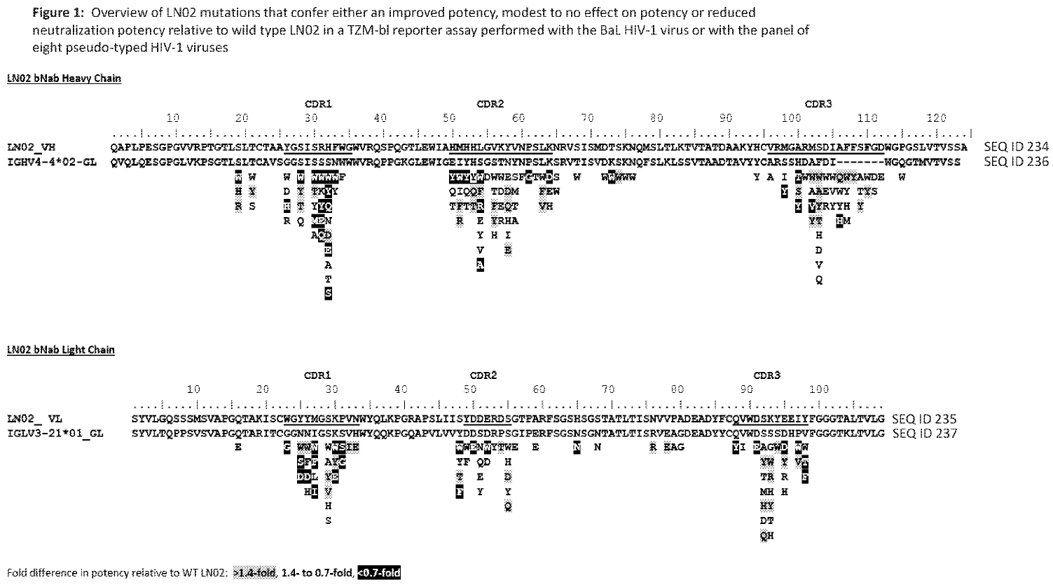

The binding agents may be antibodies such as monoclonal antibodies. As shown in the examples herein, the techniques discussed below have been used to identify a fully human mAb termed “LN02”, having particular amino acid sequences and characteristics that have described here (e.g., FIGS. 1 and 6A-E) and elsewhere. Variants of the LN02 binding agents (modified LN02 binding agents (“LN02M”)) are described herein and shown in the examples. The LN02M binding agents were prepared using recombinant molecular biology techniques. In some embodiments, the LN02M binding agents are described by referencing the amino acid and/or nucleic acid sequences corresponding to the variability and/or complementarity determining regions (“CDRs”) thereof. A CDR is known in the art to comprise amino acid sequences within the variable region identified in accordance with the definitions of the Kabat, Chothia, the accumulation of both Kabat and Chothia, AbM, contact, and/or conformational definitions or any method of CDR determination well known in the art. antibody modeling software (now Accelrys®), or the “contact definition” of CDRs based on observed antigen contacts described by MacCallum et al., 1996, J. Mol. Biol., 262:732-745. In the “conformational definition” of CDRs, the positions of the CDRs may be identified as the residues that make enthalpic contributions to antigen binding (Makabe et al., 2008, Journal of Biological Chemistry, 283:1156-1166). Still other CDR boundary definitions may not strictly follow one of the above approaches, but may nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions. In one embodiment, the skilled artisan will understand the meaning and characteristics (including the amino acid sequence) of the CDRs of LN02M binding agents from this disclosure by, for instance, with reference to those illustrated in FIG. 1 (e.g., SEQ ID NOS. 234 and 235; see also SEQ ID NOS. 1 and 93) and discussed below.

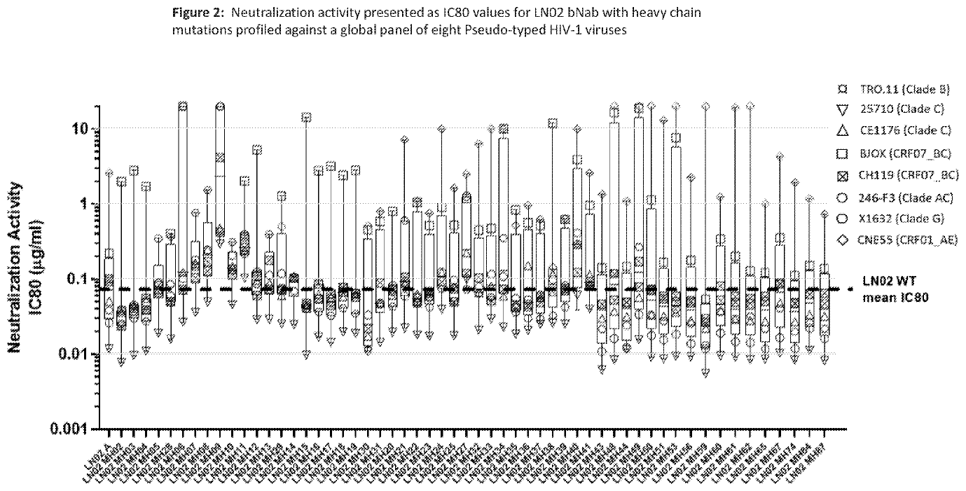

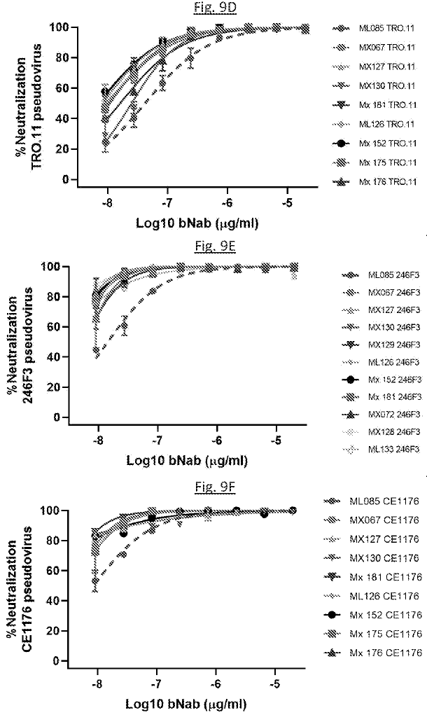

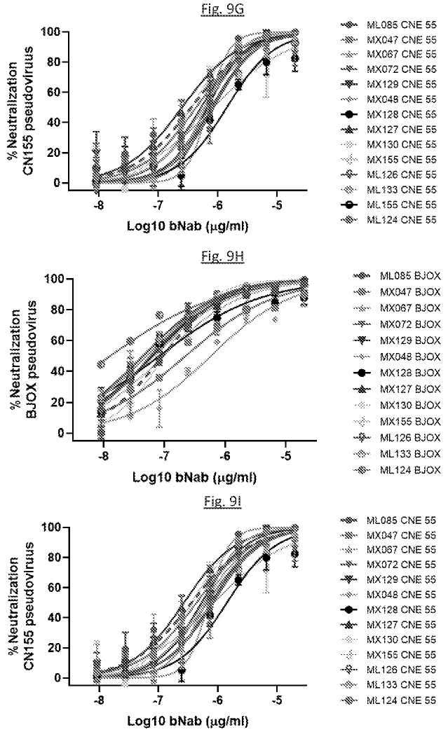

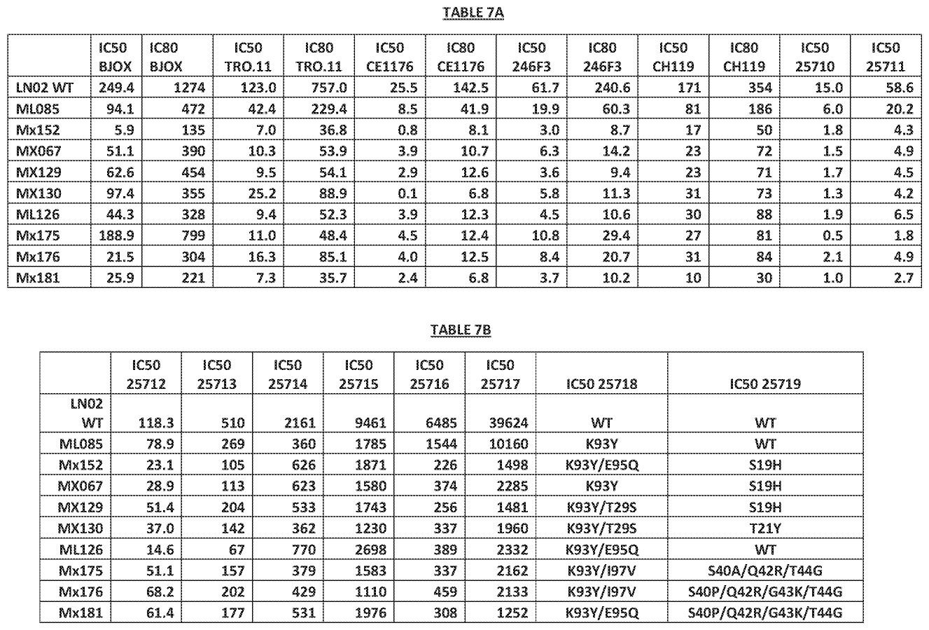

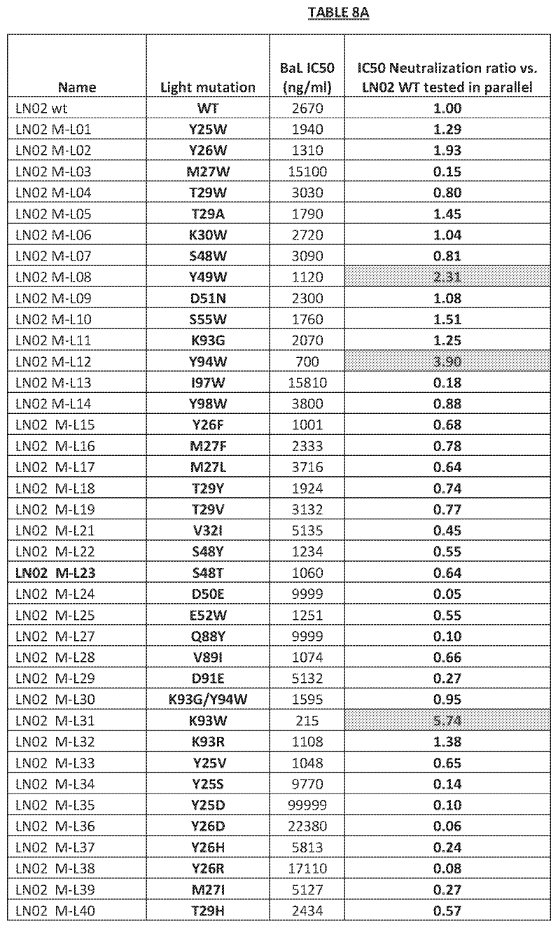

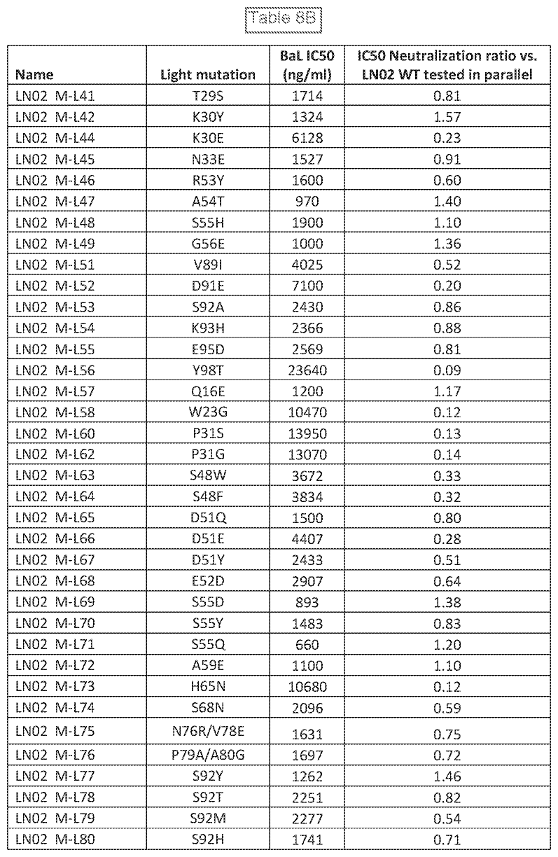

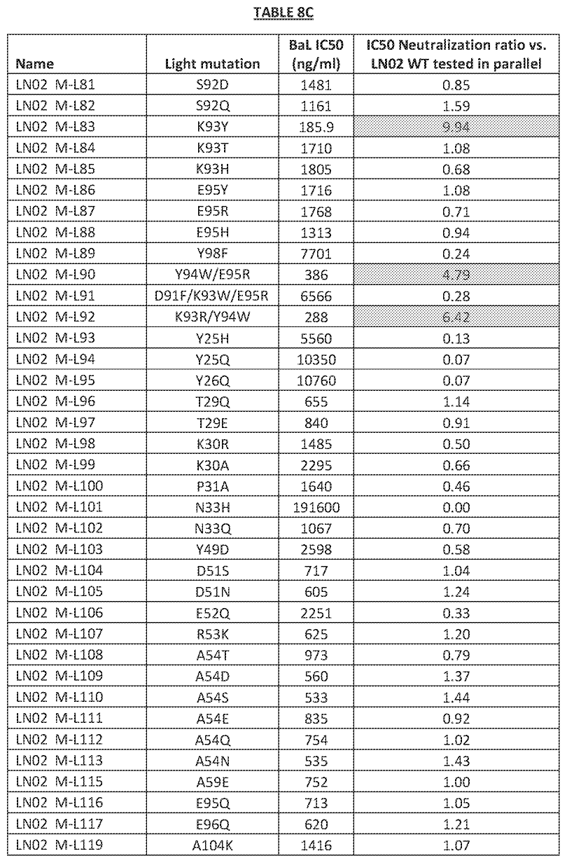

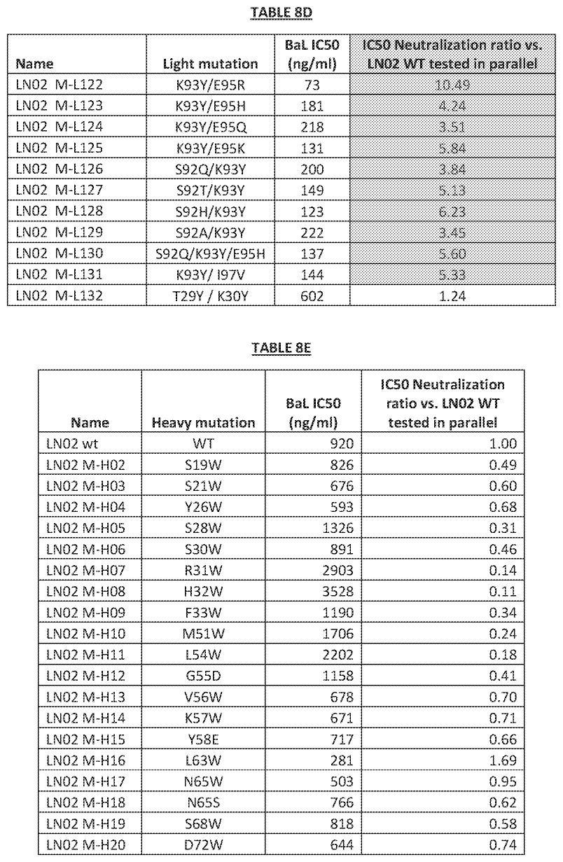

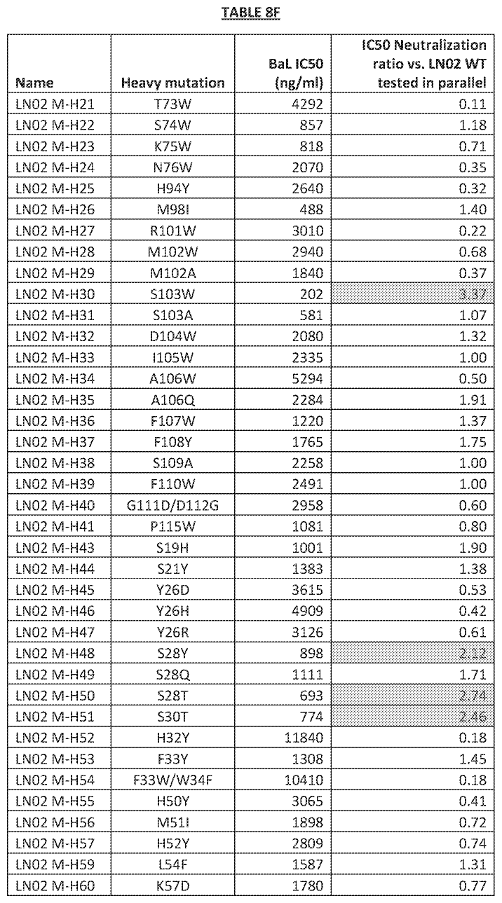

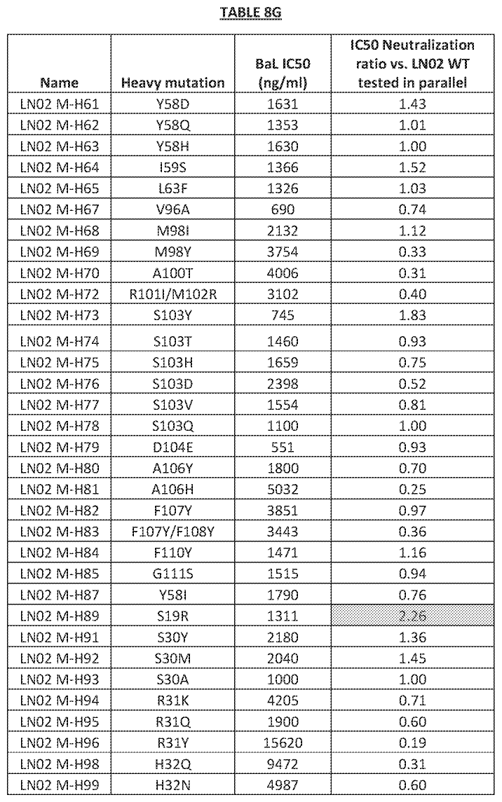

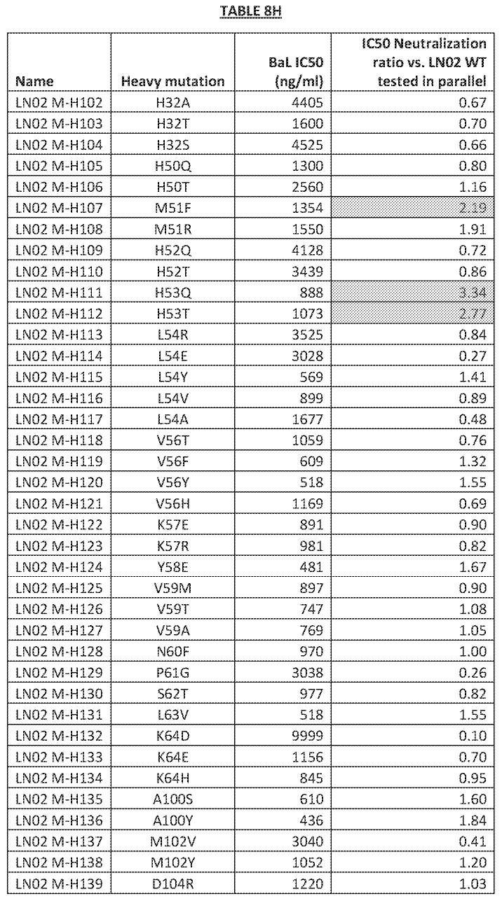

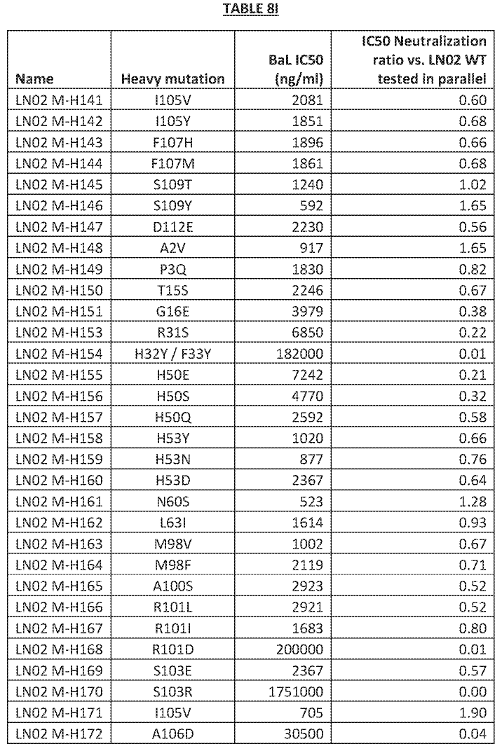

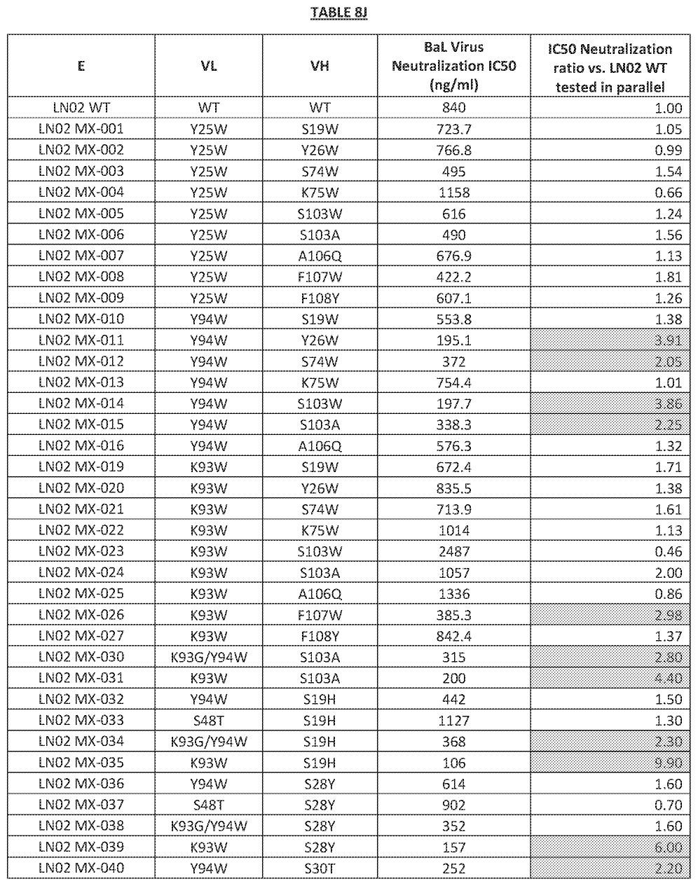

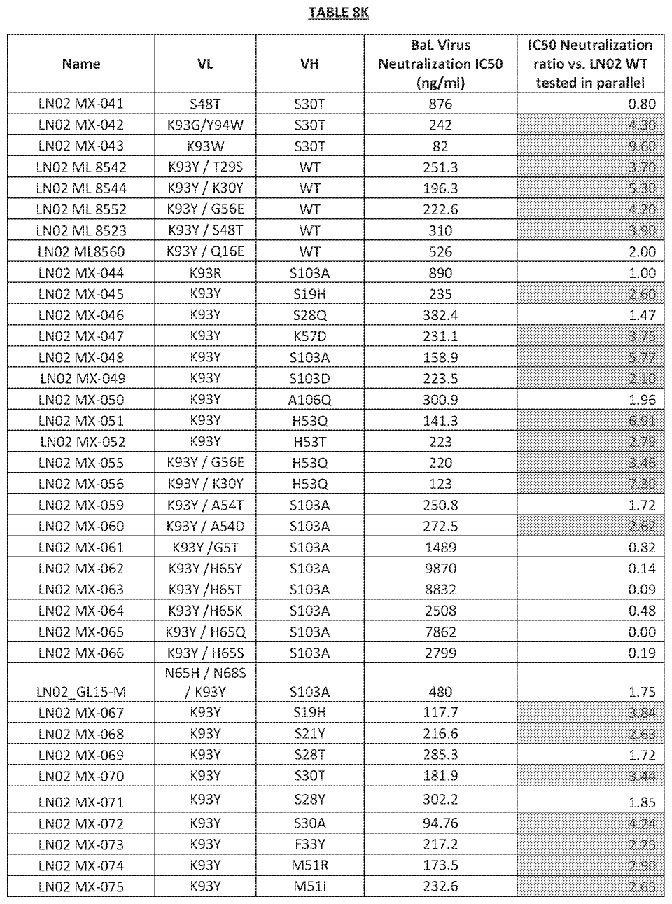

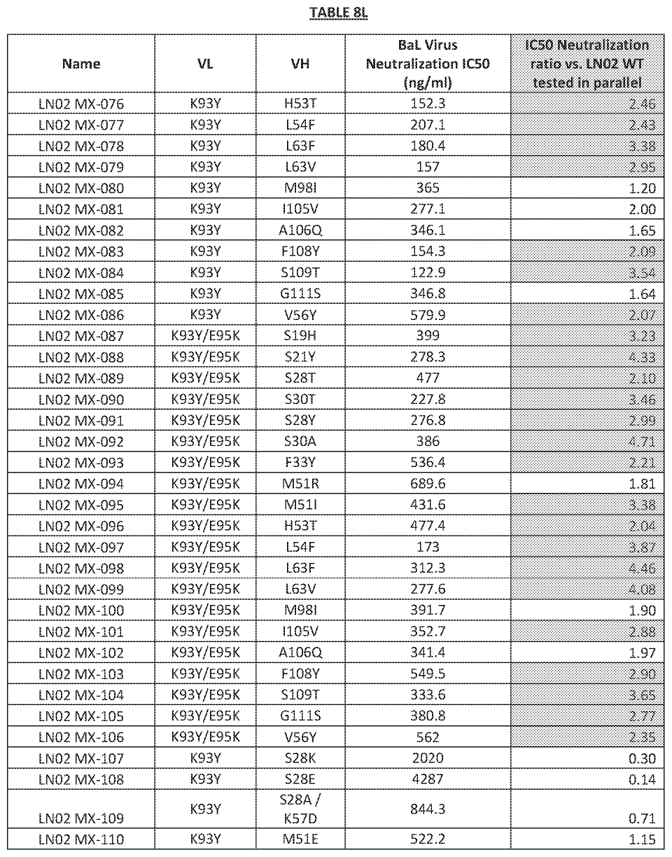

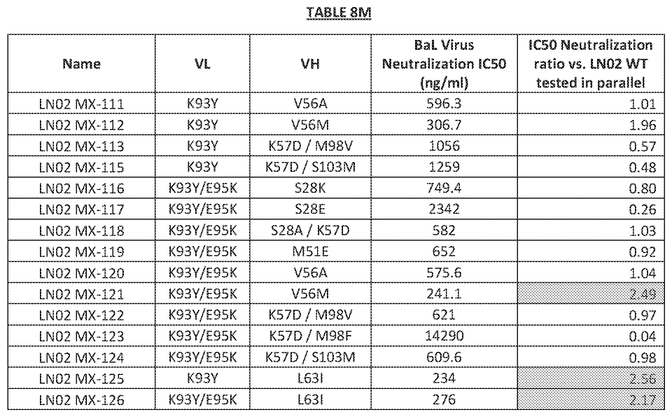

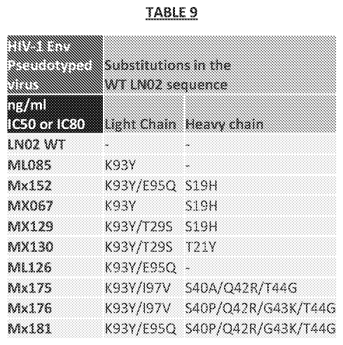

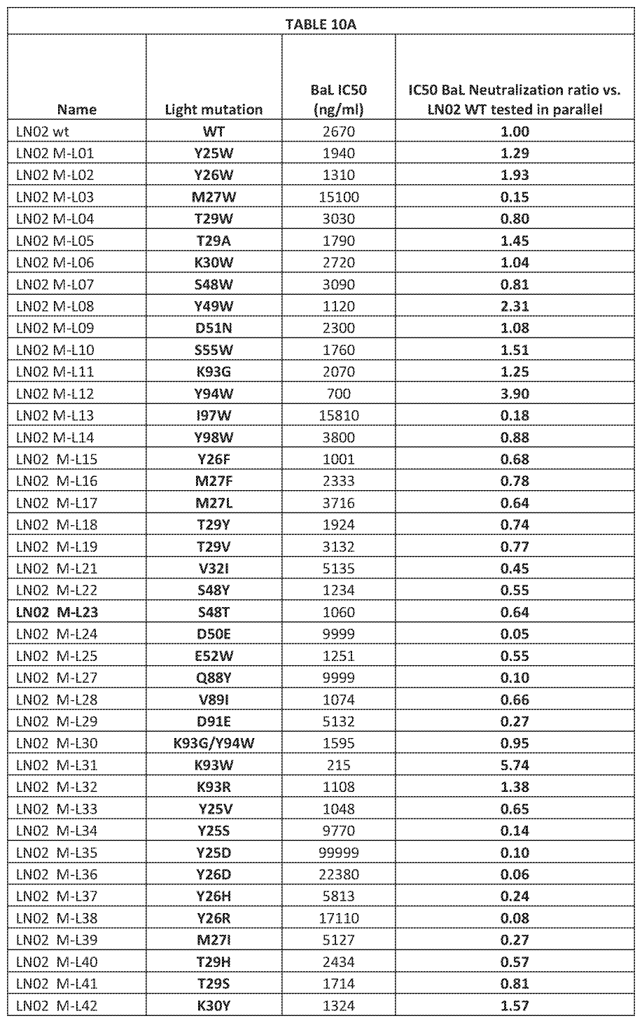

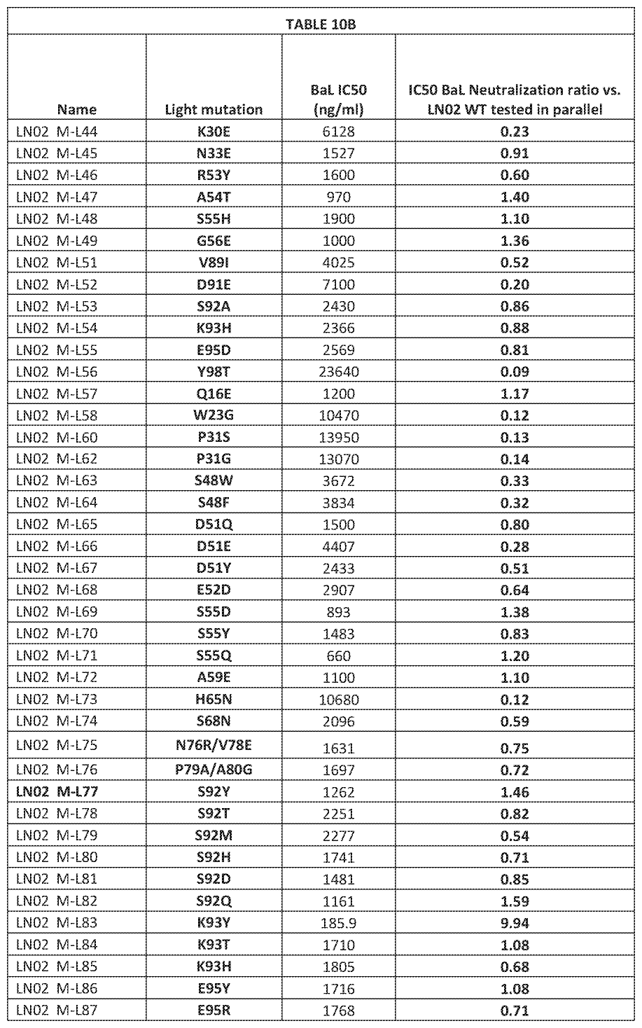

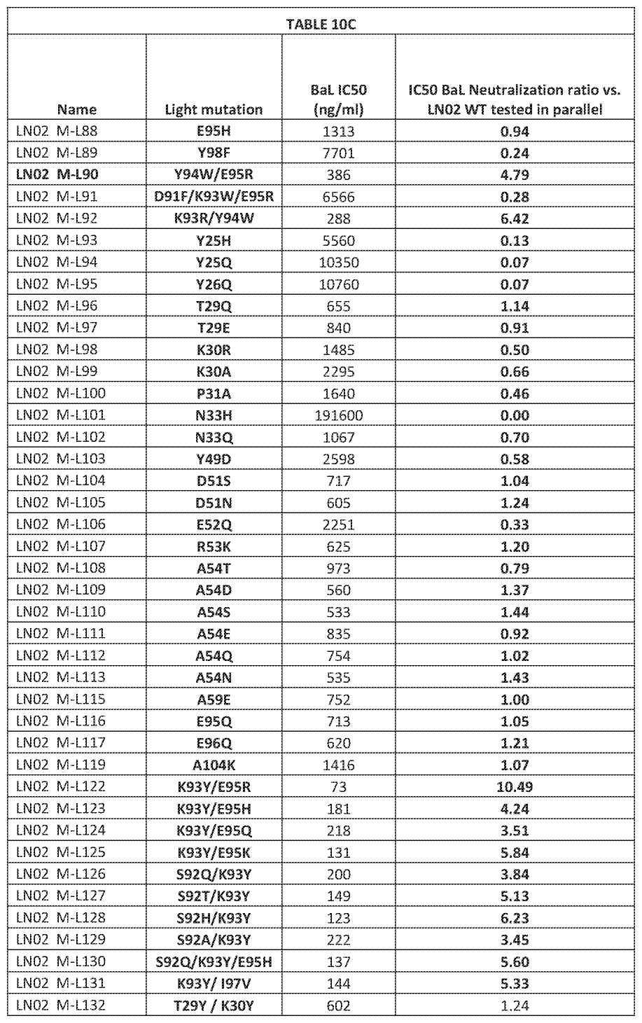

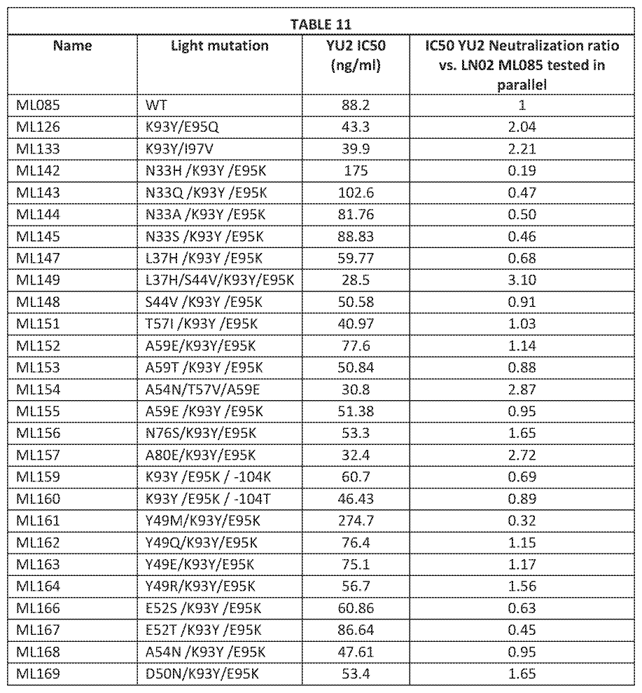

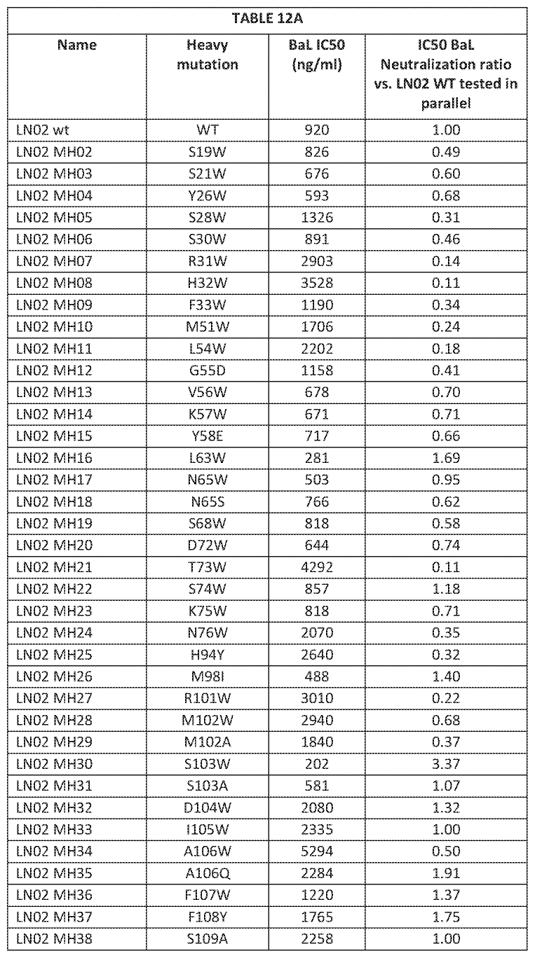

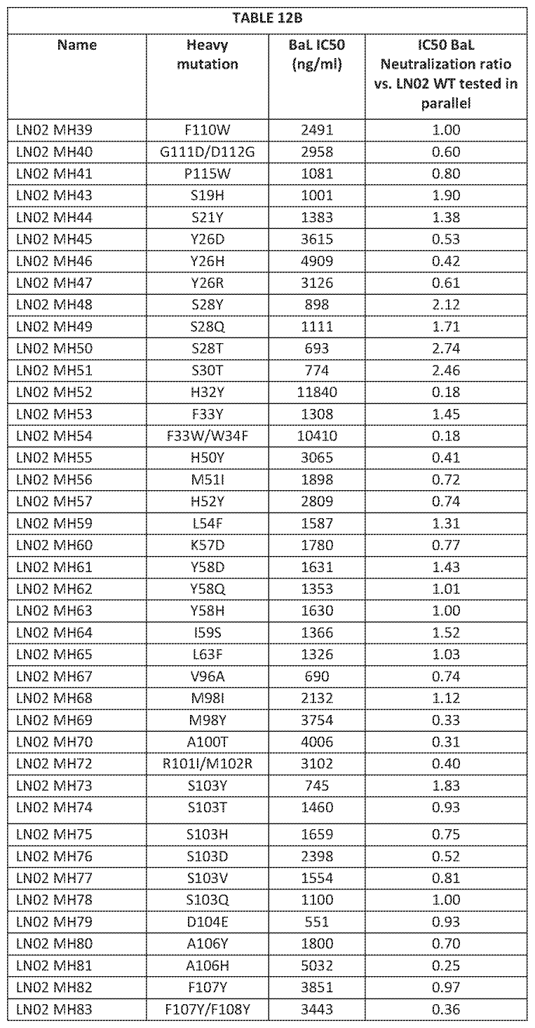

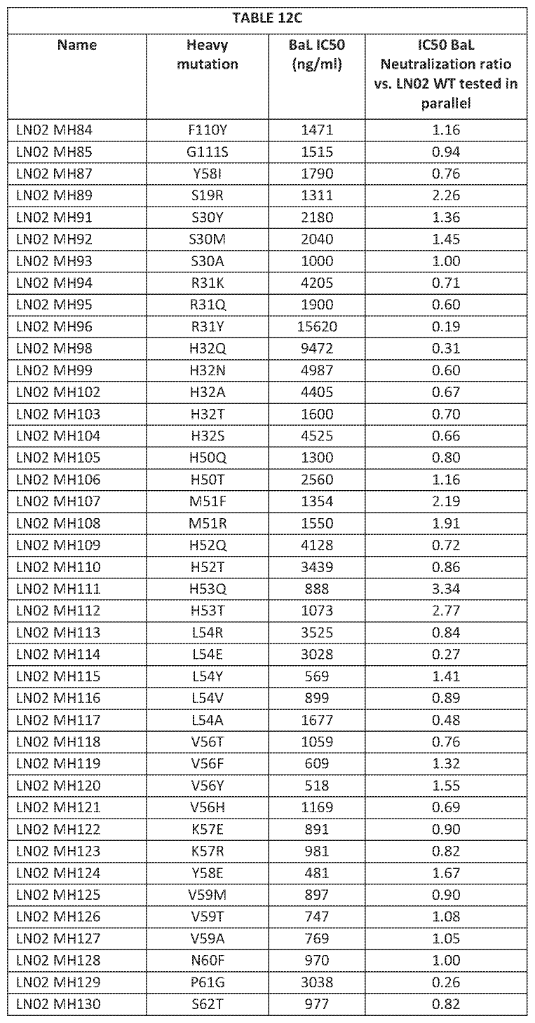

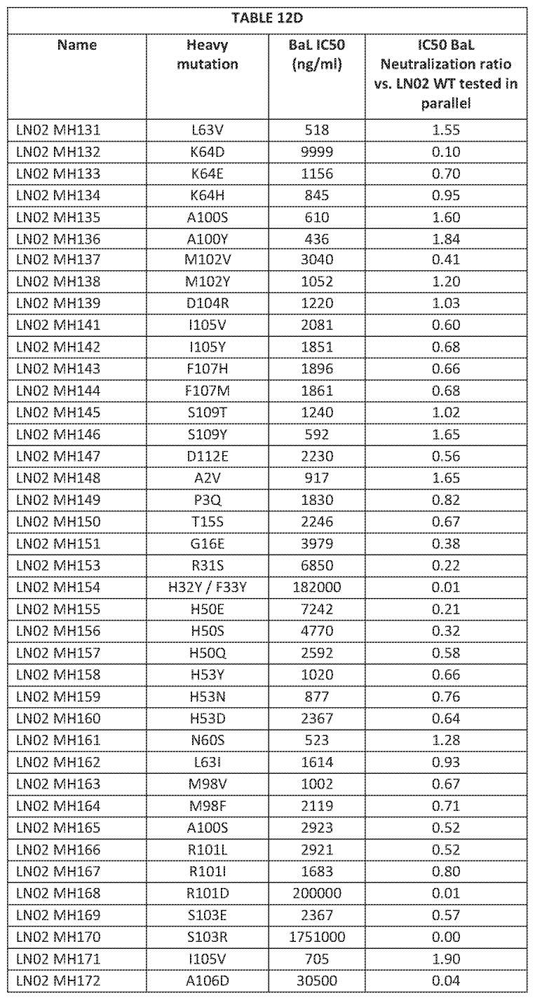

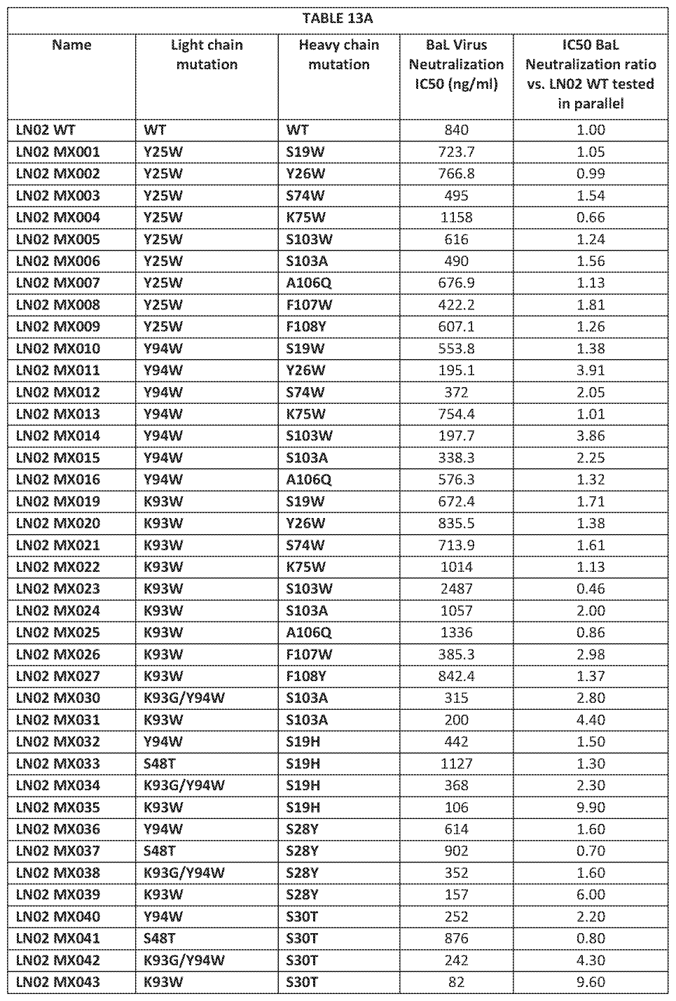

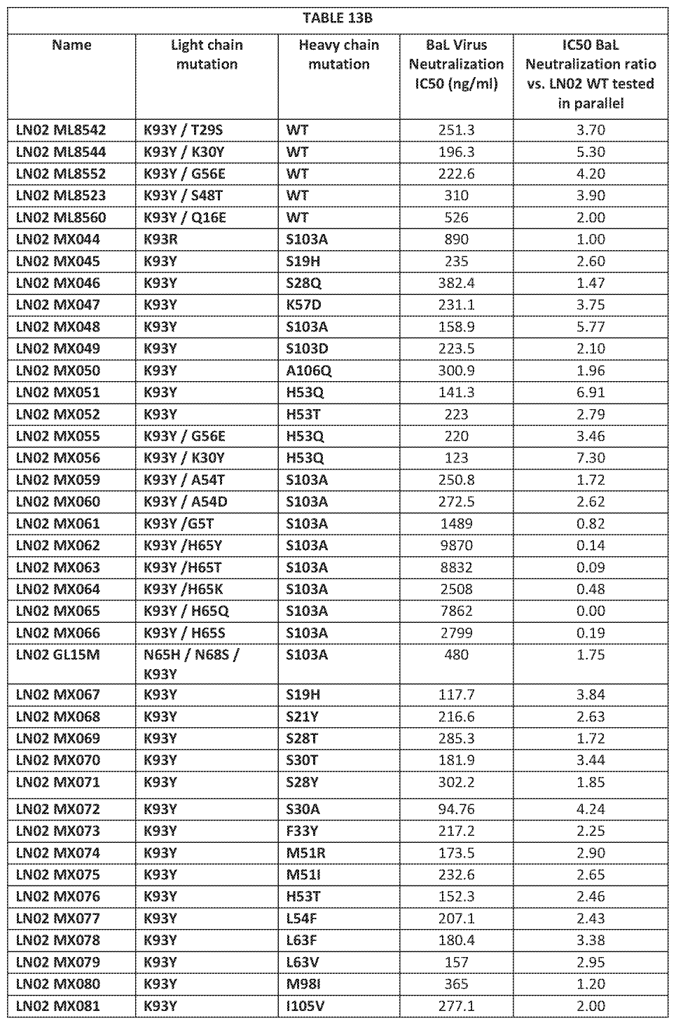

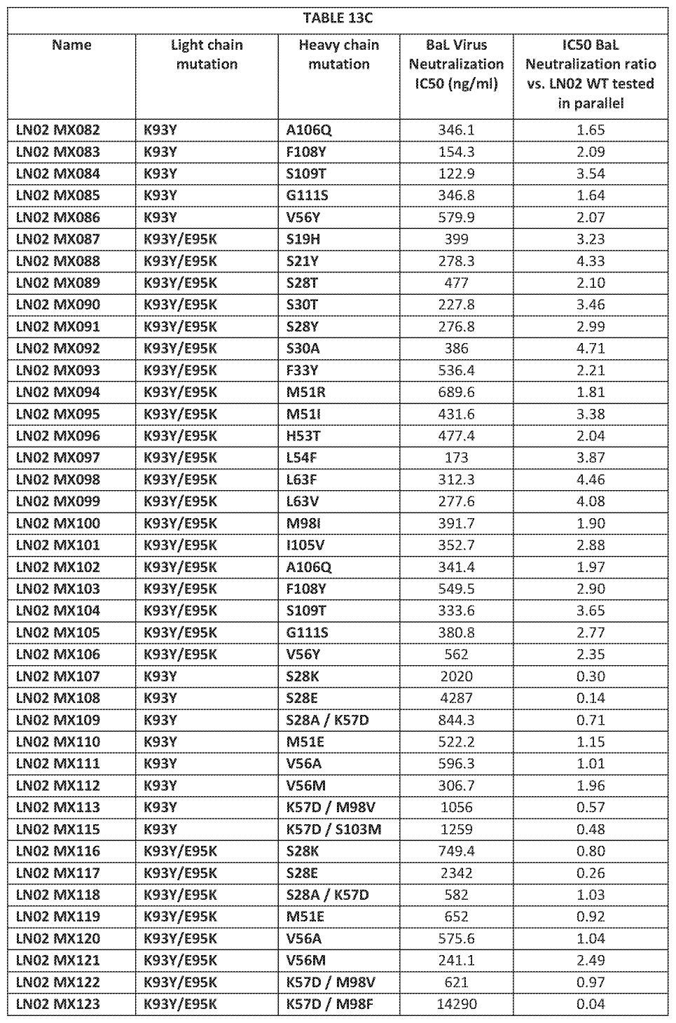

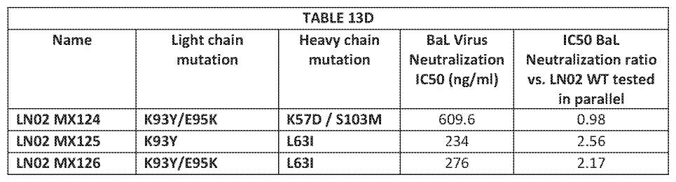

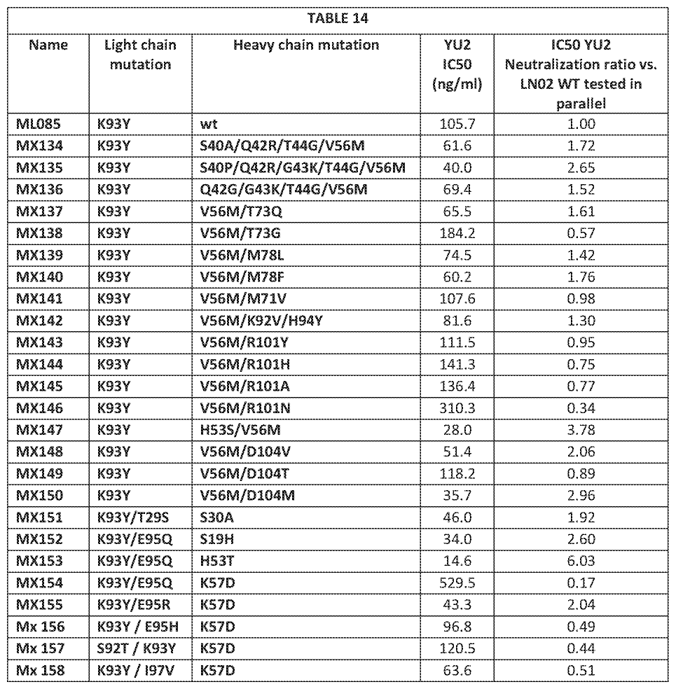

Exemplary, and preferred, amino acid sequences of the three heavy chain CDRs (CDRH1, CDRH2, CDRH3), three light chain CDRs (CDRL1, CDRL2, CDRL3), V_{H} and V_{L} domains of the LN02M binding agents (e.g., antibodies) are summarized below and in FIG. 1. Specific examples, and in some embodiments, preferred, LN02M binding agents, and/or portions thereof, are described in SEQ ID NOS. 3-92, SEQ ID NOS. 95-233 and FIGS. 6A-E. The functional phenotype (e.g., particular activities) of exemplary LN02M binding agents are also described in Tables 1-3, as well as FIGS. 2-5, and in the Examples section below. In some embodiments, the LN02M binding agents are antibodies including one or more of the CDRs described in FIG. 1 and/or FIGS. 6A-E and/or SEQ ID NOS. 3-92, and/or SEQ ID NOS. 95-233, and/or SEQ ID NOS. 248-482, and/or SEQ ID NOS. 491-699, and/or having the functional phenotype of those described in any of Tables 1-3 and/or 1-14, and/or as illustrated by FIGS. 2-5 and/or FIG. 9. In some preferred embodiments, the LN02M binding agents comprise one or more of the CDRs illustrated in FIG. 1, such as any one or more of SEQ ID NOS. 3-92, and/or SEQ ID NOS. 95-233, and the functional phenotypes described in Tables 1-3, as well as FIGS. 2-5. In some preferred embodiments, the LN02M binding agents comprise one of more of the polypeptide sequences of SEQ ID NOS. 3-92, SEQ ID NOS. 95-233, SEQ ID NOS. 248-482, and/or SEQ ID NOS. 491-699, and/or shown in FIGS. 6A-E, 7A-E, 8A-F, as well as the functional phenotypes described in any of Tables 1-3 and/or 7-14, and/or FIGS. 2-5 and/or 9.

In some embodiments, the LN02M binding agent comprises a modified LN02 CDRH1 (YGSISRHFWG), corresponding to amino acids 26-35 of the LN02_VH amino acid sequence illustrated in FIG. 1 (SEQ ID NO.: 234), and numbered left to right as Y1, G2, S3, 14, S5, R6, H7, F8, W9, and G10, may comprise any one or more, including all, or in some embodiments one or more conservative variants thereof, of the following substitutions: Y1 by W, D, H, or R; S3 by W, Y, T, or Q; S5 by W, T, Y, M or A; R6 by W, K, Y, E or Q; H7 by W, Y, Q, N, D, E, A, T or S; F8 by W or Y; and/or W9 by F. Particularly preferred substitutions to LN02 CDRH1 (YGSISRHFWG), selected on the basis of HIV neutralization potency relative to wild-type LN02 (i.e., >1.4-fold increase (e.g., FIG. 1)), are S3 to Y or T; S5 to T; and/or, H7 to D.

In some embodiments, the LN02M binding agent comprises a modified LN02 CDRH2 (HMHHLGVKYVNPSLK), corresponding to amino acids 50-64 of the LN02_VH amino acid sequence illustrated in FIG. 1 (SEQ ID NO.: 234), and numbered left to right as H1, M2, H3, H4, L5, G6, V7, K8, Y9, V10, N11, P12, S13, L14 and K15, may comprise any one or more, including all, or in some embodiments one or more conservative variants thereof, of the following substitutions: H1 by Y, Q or T; M2 by W, I, F or R; H3 by Y, Q or T; H4 by Y, Q or T; L5 by W, F, R, E, Y, V or A; G6 by D; V7 by W, T, F, Y or H; K8 by W, D, E or R; Y9 by D, Q, H, I or E; V10 by S, M, T or A; N11 by F; P12 by G; S13 by T; L14 by W, F or V; and/or, K15 by D, E or H. Particularly preferred substitutions to LN02 CDRH2 (HMHHLGVKYVNPSLK), selected on the basis of HIV neutralization potency relative to wild-type LN02 (i.e., >1.4-fold increase (e.g., FIG. 1)), are M2 by F or R; H4 by Q or T; V7 by F or Y; Y9 by D, Q, or E; and/or L14 by F or V.

In some embodiments, the LN02M binding agent comprises a modified LN02 CDRH3 (VRMGARMSDIAFFSFGD), corresponding to amino acids 96-112 of the LN02M_VH amino acid sequence illustrated in FIG. 1 (SEQ ID NO.: 234), and numbered left to right as V1, R2, M3, G4, A5, R6, M7, S8, D9, 110, A11, F12, F13, S14, F15, G16, and D17, may comprise any one or more, including all, or in some embodiments one or more conservative variants thereof, of the following substitutions: V1 by A; M3 by I or Y; A5 by T, S or Y; R6 by W; M7 by W, A, V or Y; S8 by W, A, Y, T, H, D, V or Q; D9 by W, E or R; 110 by W, V or Y; A11 by Q, W, Y or H; F12 by W, Y, H or M; F13 by Y; S14 by A, T or Y; F15 by W or Y; G16 by D or S; and/or, D17 by E. Particularly preferred substitutions to LN02 CDRH3 (VRMGARMSDIAFFSFGD), selected on the basis of HIV neutralization potency relative to wild-type LN02 (i.e., >1.4-fold increase (e.g., FIG. 1)), are A5 by S; M7 by W or Y; S8 by W, A, Y, or T; A11 by Q; F12 by W; F13 by Y; S14 by Y; and/or F15 by Y.

In some embodiments, the LN02M binding agent comprises a modified LN02 CDRL1 (WGYYMGSKPVN), corresponding to amino acids 23-33 of the LN02M_VL sequence illustrated in FIG. 1 (SEQ ID NO.: 235), and numbered left to right as W1, G2, Y3, Y4, M5, G6, S7, K8, P9, V10, and N11, may comprise any one or more, including all, or in some embodiments one or more conservative variants thereof, of the following substitutions: W1 by G; Y3 by W, S or D; Y4 by W, F, D or H; M5 by W, F, L or I; S7 by W, A, Y, V, H or S; K8 by W, Y or E; P9 by S or G; V10 by I; and/or, N11 by E. Particularly preferred substitutions to LN02 CDRL1 (WGYYMGSKPVN), selected on the basis of HIV neutralization potency relative to wild-type LN02 (i.e., >1.4-fold increase (e.g., FIG. 1)), are Y3 by W; Y4 by W; S7 by Y or V; K8 by Y; V10 by I; and/or, N11 by E.

In some embodiments, the LN02M binding agent comprises a modified LN02 CDRL2 (YDDERDS), corresponding to amino acids 49-55 of the LN02M_VL sequence illustrated in FIG. 1 (SEQ ID NO.: 235), and numbered left to right as Y1, D2, D3, E4, R5, D6, and S7, may comprise any one or more, including all, or in some embodiments one or more conservative variants thereof, of the following substitutions: Y1 by W or F; D2 by E; D3 by N, Q, E or Y; E4 by W or D; R5 by Y; D6 by T; and S7 by W, H, D, Y or Q. Particularly preferred substitutions to LN02 CDRL2 (YDDERDS), selected on the basis of HIV neutralization potency relative to wild-type LN02 (i.e., >1.4-fold increase (e.g., FIG. 1)), are D6 by T; and/or S7 by D or Q.

In some embodiments, the LN02M binding agent comprises a modified LN02 CDRL3 (QVWDSKYEEIY), corresponding to amino acids 88-98 of the LN02M_VL sequence illustrated in FIG. 1 (SEQ ID NO.: 235), and numbered left to right as CDRL3 Q1, V2, W3, D4, S5, K6, Y7, E8, E9, 110, and Y11, may comprise any one or more, including all, or in some embodiments one or more conservative variants thereof, of the following substitutions: Q1 by Y; V2 by I; D4 by E; S5 by A, Y, T, M, H, D and Q; K6 by G, W, R, H, Y, Tor H; Y7 by W; E8 by D, Y, R or H; 110 by W or V; and Y11 by W, Tor F. Particularly preferred substitutions to LN02 CDRL3 (QVWDSKYEEIY), selected on the basis of HIV neutralization potency relative to wild-type LN02 (i.e., >1.4-fold increase (e.g., FIG. 1)), are S5 by Y, H, or Q; K6 by W, or Y; Y7 by W; E8 by Y; and/or 110 by V.

In some embodiments, an LN02M binding agent can comprise modifications to the LN02 variable heavy and/or variable light chain regions amino acid sequences outside the CDRs, as those CDRs are illustrated in FIG. 1. For instance, in some embodiments, with reference to SEQ ID NO. 234, S19 can be substituted by W, H or R; T21 can be substituted by W, Y or S; S68, D72, T73, S74, K75, or N76 can be substituted by W; N65 can be substituted by S or W; H94 can be substituted by Y; and/or P105 can be substituted by W. Particularly preferred substitutions to the LN02 variable heavy chain (with reference to SEQ ID NO. 234) outside of the CDRs, selected on the basis of HIV neutralization potency relative to wild-type LN02 (i.e., >1.4-fold increase (e.g., FIG. 1)), are S19 by H or R; T21 by Y; and/or S74 by W. In some embodiments, with reference to SEQ ID NO. 235, Q16 can be substituted by E; S48 can be substituted by W, Y, T or F; G56 can be substituted by E; A59 can be substituted by E; H65 can be substituted by N; S68 can be substituted by N; N76 can be substituted by R; V78 can be substituted by E; P79 can be substituted by A; and/or, A80 can be substituted by G. Particularly preferred substitutions to the LN02 variable light chain (with reference to SEQ ID NO. 235) outside of the CDRs, selected on the basis of HIV neutralization potency relative to wild-type LN02 (i.e., >1.4-fold increase (e.g., FIG. 1)), are S48 by T; N76 by R; and/or V78 by E. Any one of more of these substitutions to the LN02 variable heavy and variable light chains (with reference to SEQ ID NOS. 234 and 235, respectively) outside of the CDRs can be included in a binding agent comprising any of the LN02M CDRs described above. Additional conservative substitutions to such amino acid sequences can also be utilized as would be understood by those of ordinary skill in the art.

In some embodiments, an LN02M binding agent can comprise an LN02M polypeptide modified as illustrated in FIG. 1. In some embodiments, the LN02M binding agent can be a polypeptide comprising the amino acid sequences described in any of FIGS. 6A-E. Preferred LN02M polypeptides, based on the potency data presented in Tables 1-2, are those exhibiting greater neutralizing activity than wild-type LN02 (fold increase indicated in parenthesis), including those comprising the LN02M variable heavy chain regions MH01 (1.59) (SEQ ID NO. 95), MH16 (1.69) (SEQ ID NO. 110), MH22 (1.18) (SEQ ID NO. 116), MH26 (1.40) (SEQ ID NO. 120), MH30 (3.37) (SEQ ID NO. 124), MH32 (1.32) (SEQ ID NO. 126), MH35 (1.91) (SEQ ID NO. 129), MH36 (1.37) (SEQ ID NO. 130), MH37 (1.75) (SEQ ID NO. 131), MH43 (1.90) (SEQ ID NO. 136), MH44 (1.38) (SEQ ID NO. 137), MH48 (2.12) (SEQ ID NO. 141), MH49 (1.71) (SEQ ID NO. 142), MH50 (2.74) (SEQ ID NO. 143), MH51 (2.46) (SEQ ID NO. 144), MH53 (1.45) (SEQ ID NO. 146), MH59 (1.31) (SEQ ID NO. 151), MH61 (1.43) (SEQ ID NO. 153), MH64 (1.52) (SEQ ID NO. 156), MH68 (1.12) (SEQ ID NO. 159), MH73 (1.83) (SEQ ID NO. 163), MH84 (1.16) (SEQ ID NO. 174), MH89 (2.26) (SEQ ID NO. 177), MH91 (1.36) (SEQ ID NO. 178), MH92 (1.45) (SEQ ID NO. 179), MH106 (1.16) (SEQ ID NO. 193), MH107 (2.19) (SEQ ID NO. 194), MH108 (1.91) (SEQ ID NO. 195), MH111 (3.34) (SEQ ID NO. 198), MH112 (2.77) (SEQ ID NO. 199), MH115 (1.41) (SEQ ID NO. 202), MH119 (1.32) (SEQ ID NO. 206), MH120 (1.55) (SEQ ID NO. 207), MH124 (1.67) (SEQ ID NO. 211), MH 131 (1.55) (SEQ ID NO. 218), MH135 (1.60) (SEQ ID NO. 222), MH136 (1.84) (SEQ ID NO. 223), MH138 (1.20) (SEQ ID NO. 225), and/or MH146 (1.65) (SEQ ID NO. 232); and/or the LN02M variable light chain regions ML01 (1.29) (SEQ ID NO. 3), ML02 (1.93) (SEQ ID NO. 4), ML05 (1.45) (SEQ ID NO. 7), ML08 (2.31) (SEQ ID NO. 10), ML10 (1.51) (SEQ ID NO. 12), ML11 (1.25) (SEQ ID NO. 13), ML12 (3.90) (SEQ ID NO. 14), ML31 (5.74) (SEQ ID NO. 31), ML32 (1.38) (SEQ ID NO. 32), ML44 (1.57) (SEQ ID NO. 42), ML49 (1.40) (SEQ ID NO. 47), ML51 (1.10) (SEQ ID NO. 48), ML52 (1.36) (SEQ ID NO. 49), ML60 (1.17) (SEQ ID NO. 56), ML71 (1.38) (SEQ ID NO. 66), ML73 (1.20) (SEQ ID NO. 68), ML74 (1.10) (SEQ ID NO. 69), ML79 (1.46) (SEQ ID NO. 74), ML84 (1.59) (SEQ ID NO. 79), ML85 (9.94) (SEQ ID NO. 80), ML92 (4.79) (SEQ ID NO. 87), and ML94 (6.42) (SEQ ID NO. 89); and/or conservatively substituted variants and/or fragments thereof. Particularly preferred LN02M binding agents are those comprising LN02 MH30 (SEQ ID NO. 95), LN02 MH111 (SEQ ID NO. 95), LN02 ML12 (SEQ ID NO. 95), LN02 ML31 (SEQ ID NO. 95), LN02 ML85 (SEQ ID NO. 95), LN02 ML92 (SEQ ID NO. 95), and LN02 ML94 (SEQ ID NO. 95), each of which exhibits greater than three-fold improved neutralization potency against the BaL virus relative the LN02 wild type control (Tables 1-2), and/or conservatively substituted variants and/or fragments thereof.

A binding agent of this disclosure can comprise any of the modified CDRH1, CDRH2, CDRH3, CDRL1, CDRL2, and CDRL3 amino acid sequences described above, or conservatively substituted variants thereof. Such a binding agent may be a polypeptide, such as an antibody, as described in more detail below.

A binding agent of this disclosure may comprise, for example, any one or more of the amino acid sequences (i.e., polypeptide sequences) of SEQ ID NOS. 3-92, SEQ ID NOS. 95-233, and/or those shown in FIGS. 1 and 6A-E; and/or or a conservatively substituted variant thereof. Fragments and/or derivatives (e.g., comprising substituted amino acids, such as conservative substitutions) thereof are also disclosed. In some embodiments, a binding agent of this disclosure may comprise one or more (i.e., one, two, three, four, five, six or seven) of SEQ ID NOS. 3-92, SEQ ID NOS. 95-233, and/or those shown in FIGS. 6A-E, provided the binding agent exhibits the functional characteristics described herein (e.g., as shown in FIGS. 2-5 and the Examples section). In preferred embodiments, the binding agent comprises at least one of the modified CDRs illustrated in FIG. 1; at least one of SEQ ID NOS. 3-92 and at least one of SEQ ID NOS. 95-233; and/or at least one of the amino acid sequences illustrated in FIGS. 6A-E; even more preferably wherein such LN02M binding agents exhibit one or more of the properties presented in any of Tables 1-3 and/or FIGS. 2-5, and/or described in the Examples section herein. Together, the modified LN02 amino acid sequences disclosed herein can be referred to as the “LN02M variable region and/or CDR and/or non-CDR amino acid sequences”, which refers to the LN02M amino acid sequences illustrated in FIGS. 1 and 6A-E as well as SEQ ID NOS. 3-92 and 95-233. The LN02M variable region and/or CDR and/or non-CDR amino acid sequences typically include a sequence of at least six amino acid residues (e.g., a sequence of at least any of seven, eight, nine, ten, 11, 12, 13, 14 or 15 amino acid residues).

Combinations of LN02 CDRs with LN02M CDRs, as well as modified amino acid sequences outside of CDRs, can be combined with one another as desired, typically while maintaining the functional characteristics such as HIV pseudo-virus neutralization (as illustrated by Tables 1-3 and 7-14, FIGS. 2-5 and 9, and the Examples). Exemplary combinations are described in Table 4, and also include combinations of: LN02M variable light chain ML01 (SEQ ID NO. 3) with LN02M variable heavy chain MH02 (SEQ ID NO. 96), MH04 (SEQ ID NO. 98), MH22 (SEQ ID NO. 116), MH23 (SEQ ID NO. 117), MH30 (SEQ ID NO. 124), MH31 (SEQ ID NO. 125), MH35 (SEQ ID NO. 129), MH36 (SEQ ID NO. 130), and/or MH37 (SEQ ID NO. 131); LN02M variable light chain ML12 (SEQ ID NO. 14) with LN02M variable heavy chain MH02 (SEQ ID NO. 96), MH04 (SEQ ID NO. 98), MH22 (SEQ ID NO. 116), MH23 (SEQ ID NO. 117) MH30 (SEQ ID NO. 124), MH31 (SEQ ID NO. 125), MH35 (SEQ ID NO. 129), MH36 (SEQ ID NO. 130), and/or MH37 (SEQ ID NO. 131); LN02M variable light chain ML23 (SEQ ID NO. 24) with LN02M variable heavy chain MH31 (SEQ ID NO. 125), MH43 (SEQ ID NO. 136), MH48 (SEQ ID NO. 141), and MH51 (SEQ ID NO. 144); LN02M variable light chain ML30 (SEQ ID NO. 30) with LN02M variable heavy chain MH31 (SEQ ID NO. 125), MH43 (SEQ ID NO. 136), MH48 (SEQ ID NO. 141), or MH51 (SEQ ID NO. 144); LN02M variable light chain ML31 (SEQ ID NO. 31) with LN02M variable heavy chain MH02 (SEQ ID NO. 96), MH04 (SEQ ID NO. 98), MH22 (SEQ ID NO. 116), MH23 (SEQ ID NO. 117), MH30 (SEQ ID NO. 124), MH31 (SEQ ID NO. 125), MH35 (SEQ ID NO. 129), MH36 (SEQ ID NO. 130), MH37 (SEQ ID NO. 131), MH43 (SEQ ID NO. 136), MH48 (SEQ ID NO. 141), or MH51 (SEQ ID NO. 144); LN02M variable light chain ML32 (SEQ ID NO. 32) with LN02M variable heavy chain MH31 (SEQ ID NO. 125); LN02M variable light chain ML85 (SEQ ID NO. 80) with LN02M variable heavy chain MH31 (SEQ ID NO. 125), MH35 (SEQ ID NO. 129), MH43 (SEQ ID NO. 136), MH49 (SEQ ID NO. 142), MH60 (SEQ ID NO. 152), MH76 (SEQ ID NO. 166), MH111 (SEQ ID NO. 198), or MH112 (SEQ ID NO. 199); and/or conservatively substituted variants and/or fragments thereof. Particularly preferred LN02M binding agents are LN02 ML8542 (SEQ ID NO. 99) and LN02 MX048 (a combination of LN02 ML85 (SEQ ID NO. 80) and LN02 MH31 (SEQ ID NO. 125) (Table 4); and/or conservatively substituted variants and/or fragments thereof. Other combinations are also contemplated herein as will be understood by those of ordinary skill in the art.

In some embodiments, the binding agent can be a monoclonal antibody (mAb) or a fragment or derivative thereof. In some embodiments, the binding agent may be an HIV-binding fragment of such a monoclonal antibody (mAb). In some embodiments, one or more LN02M CDR(s), and/or an amino acid sequence comprising such CDR(s), and in some embodiments other modified sequences present outside the CDR regions (see, e.g., FIG. 1) cloned into an IgG (e.g., IgG1 or IgG3) backbone (e.g., framework) using standard techniques. Other suitable embodiments may be derived by those of ordinary skill in the art from this disclosure.

It is preferred that the binding agent (e.g., antibody, or the antigen binding fragment thereof), comprises one or more amino acid sequences having at least 70%, at least 75%, at least 80%, at least 85%, at least 88%, at least 90%, at least 92%, at least 95%, at least 96%, at least 97%, at least 98% or at least 99% identity to at least one LN02M variable region and/or CDR and/or non-CDR amino acid sequences (e.g., the modified CDRs illustrated in FIG. 1; at least one of SEQ ID NOS. 3-92 or 491-699 and at least one of SEQ ID NOS. 95-233 or 248-482; and/or at least one of the amino acid sequences illustrated in FIG. 6A-E, 7A-E, or 8A-F), and even more preferably wherein a LN02M binding agent further exhibits one or more of the properties presented in any of Tables 1-3 and/or 7-14 and/or FIGS. 2-5 and/or 9, and/or described in the Examples section herein. As discussed below, identities of less than 100% may result from the natural or synthetic substitution of one or more amino acids with another amino acid(s), as in a conservative substitution (see, e.g., Table 4). Various combinations of the LN02M variable region and/or CDR and/or non-CDR amino acid sequences are also contemplated and can be useful for similar purposes (e.g., as an anti-HIV antibody) as may ascertained by one of ordinary skill in the art using the techniques described herein or as may be otherwise available to those of ordinary skill in the art. In preferred embodiments, the LN02M binding agents described herein can bind HIV and/or cells infected by HIV and/or expressing HIV proteins. In some especially preferred embodiments, the LN02M binding agents described herein can neutralize HIV (e.g., perform as neutralizing binding agents (e.g., antibodies)). In preferred embodiments, the LN02M binding agents can both bind HIV and/or cells infected by HIV and/or expressing HIV proteins, and neutralize HIV.

The LN02M variable region and/or LN02M CDR and/or non-CDR amino acid sequences may be used in combination with one or more other variable region/CDR amino acid sequences available to those of ordinary skill in the art. Such variable region/CDR amino acid sequences may alternatively and/or also be adjoined to one or more types of constant region polypeptides of an antibody molecule. For instance, the LN02M CDRs can be adjoined to or associated with the constant regions of any antibody molecule of the same or a different species (e.g., human, goat, rat, sheep, chicken) and/or antibody subtype of that from which the CDR amino acid sequence was derived. For instance, an exemplary binding agent LN02M may be, or may be derived from, one having about the same neutralizing activity and/or binding the same or similar epitopes and/or exhibiting about the same affinity as another binding agent comprising one or more LN02M variable region and/or CDR and/or non-CDR amino acid sequences. The binding agent may comprise an antibody heavy and/or a light chain that each comprises one or more constant and/or variable regions. Any of the amino acid sequences described herein (e.g., the LN02M variable region and/or CDR and/or non-CDR amino acid sequences), and/or any fragments and/or derivatives thereof, may also be combined with any other variable region and/or CDR in any order and/or combination to form new binding agents, e.g., hybrid and/or fusion binding agents, and/or inserted into other heavy and/or light chain variable regions using standard techniques.

This disclosure also provides for the use of such binding agents to isolate, identify, and/or target HIV and/or cells harboring and/or infected by HIV and/or expressing HIV antigens. In certain embodiments, such binding agents may be reactive against HIV antigens such as proteins expressed on the surface of cells. In some embodiments, the binding agent(s) is an antibody (antibodies). The term “antibody” or “antibodies” may refer to whole or fragmented antibodies in unpurified or partially purified form (e.g., hybridoma supernatant, ascites, polyclonal antisera) or in purified form. The antibodies may be of any suitable origin or form including, for example, murine (e.g., produced by murine hybridoma cells), or expressed as humanized antibodies, chimeric antibodies, human antibodies, and the like. For instance, antibodies may be wholly or partially derived from human (e.g., IgG (IgG1, IgG2, IgG3, IgG4), IgM, IgA (IgA1 and IgA2), IgD, and IgE), canine (e.g., IgGA, IgGB, IgGC, IgGD), chicken (e.g., IgA, IgD, IgE, IgG, IgM, IgY), goat (e.g., IgG), mouse (e.g., IgG, IgD, IgE, IgG, IgM), pig (e.g., IgG, IgD, IgE, IgG, IgM), and/or rat (e.g., IgG, IgD, IgE, IgG, IgM) antibodies, for instance. Methods of preparing, utilizing and storing various types of antibodies are well-known to those of skill in the art and would be suitable in practicing the present invention (see, for example, Harlow, et al. Antibodies: A Laboratory Manual, Cold Spring Harbor Laboratory, 1988; Harlow, et al. Using Antibodies: A Laboratory Manual, Portable Protocol No. 1, 1998; Kohler and Milstein, Nature, 256:495 (1975)); Jones et al. Nature, 321:522-525 (1986); Riechmann et al. Nature, 332:323-329 (1988); Presta (Curr. Op. Struct. Biol., 2:593-596 (1992); Verhoeyen et al. (Science, 239:1534-1536 (1988); Hoogenboom et al., J. Mol. Biol., 227:381 (1991); Marks et al., J. Mol. Biol., 222:581 (1991); Cole et al., Monoclonal Antibodies and Cancer Therapy, Alan R. Liss, p. 77 (1985); Boerner et al., J. Immunol., 147(1):86-95 (1991); Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); as well as U.S. Pat. Nos. 4,816,567; 5,545,807; 5,545,806; 5,569,825; 5,625,126; 5,633,425; and, 5,661,016). In certain applications, the antibodies may be contained within hybridoma supernatant or ascites and utilized either directly as such or following concentration using standard techniques. In other applications, the antibodies may be further purified using, for example, salt fractionation and ion exchange chromatography, or affinity chromatography using Protein A, Protein G, Protein A/G, and/or Protein L ligands covalently coupled to a solid support such as agarose beads, or combinations of these techniques. The antibodies may be stored in any suitable format, including as a frozen preparation (e.g., −20° C. or −70° C.), in lyophilized form, or under normal refrigeration conditions (e.g., 4° C.). When stored in liquid form, for instance, it is preferred that a suitable buffer such as Tris-buffered saline (TBS) or phosphate buffered saline (PBS) is utilized. In some embodiments, the binding agent may be prepared as an injectable preparation, such as in suspension in a non-toxic parenterally acceptable diluent or solvent. Suitable vehicles and solvents that may be utilized include water, Ringer's solution, and isotonic sodium chloride solution, TBS and/or PBS, among others. Such preparations may be suitable for use in vitro or in vivo may be prepared as is known in the art and the exact preparation may depend on the particular application.

The binding agents described herein are not, however, in any way limited to antibodies (i.e., whole antibodies). For example, the binding agent may be any compound exhibiting similar binding properties as another (e.g., a mimetic). For example, an exemplary binding agent may be one that binds HIV and/or can compete with another binding agent having specificity therefor (e.g., a monoclonal antibody such as a LN02M antibody). In some embodiments, the mimetic may exhibit substantially the same affinity in binding assays as the binding agent (e.g., monoclonal antibody) to which it is being compared. The affinity a particular binding agent may be measured by any suitable assay including but not limited to FACS staining of cell surface HIV antigens (e.g., polypeptides). One binding agent may be said to have “substantially the same affinity” as another where the measurements (e.g., nm) are within about any of 1-20, 1-5, 5-10, 10-15, or 15-20 percent of one another. Exemplary mimetics may include, for example, organic compounds that specifically bind HIV, or an affibody (Nygren, et al. FEBS J. 275 (11): 2668-76 (2008)), affilin (Ebersbach, et al. J. Mol. Biol. 372 (1): 172-85 (2007)), affitin (Krehenbrink, et al. J. Mol. Biol. 383 (5): 1058-68 (2008)), anticalin (Skerra, A. FEBS J. 275 (11): 2677-83 (2008)), avimer (Silverman, et al. Nat. Biotechnol. 23 (12): 1556-61 (2005)), DARPin (Stumpp, et al. Drug Discov. Today 13 (15-16): 695-701 (2008)), Fynomer (Grabulovski, et al. J. Biol. Chem. 282 (5): 3196-3204 (2007)), Kunitz domain peptide (Nixon, et al. Curr. Opin. Drug Discov. Devel. 9 (2): 261-8 (2006)), and/or a monobody (Koide, et al. Methods Mol. Biol. 352: 95-109 (2007)). Other mimetics may include, for example, a derivative of an antibody such as, for example, an F_{ab}, F_{ab2}, Fab′ single chain antibody, Fᵥ, single domain antibody, mono-specific antibody, bi-specific antibody, tri-specific antibody, multi-valent antibody, chimeric antibody, canine-human chimeric antibody, canine-mouse chimeric antibody, antibody comprising a canine Fc, humanized antibody, human antibody, caninized, CDR-grafted antibody, shark antibody, nanobody, camelid antibody, microbody, and/or intrabody; and/or derivative thereof. Other binding agents are also provided herein as would be understood by one of ordinary skill in the art.

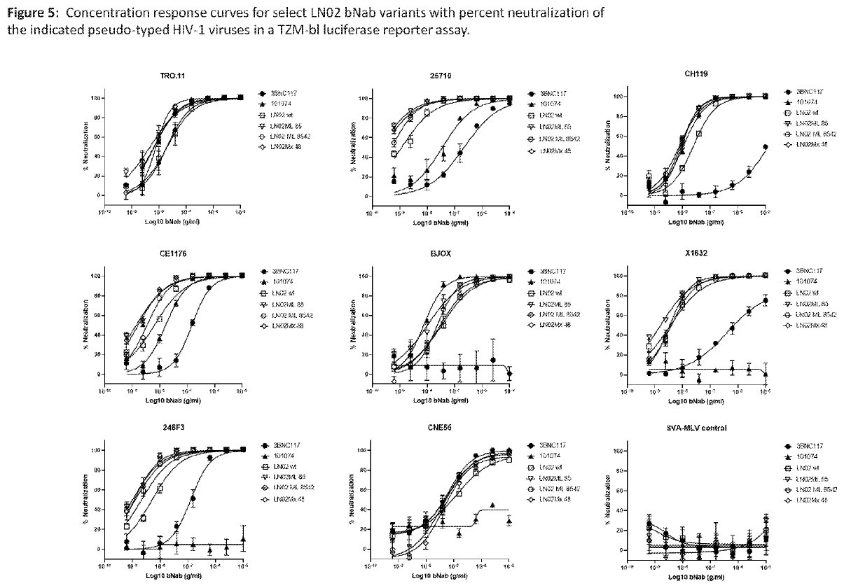

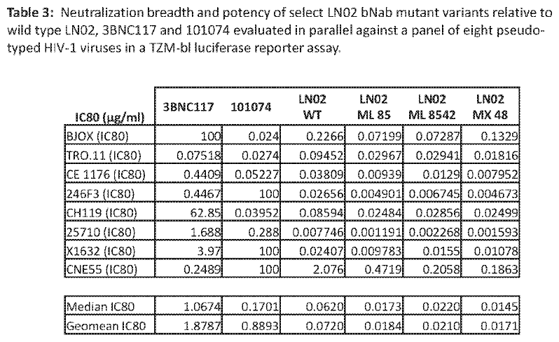

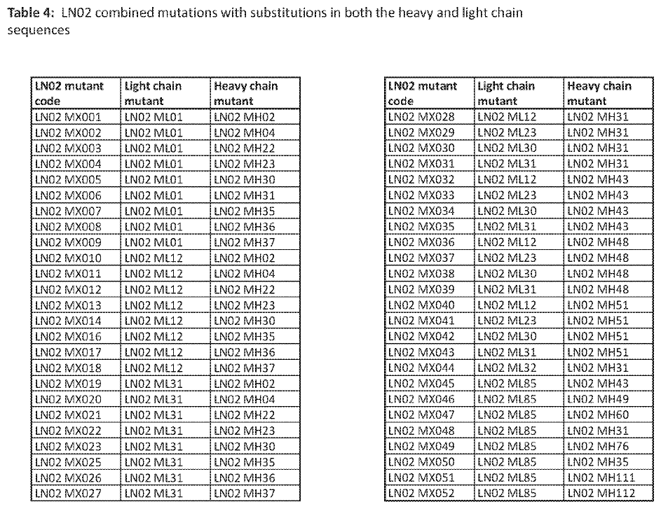

Any method known to those of ordinary skill in the art may be used to generate binding agents having specificity for (e.g., binding to) HIV. For instance, to generate and isolate monoclonal antibodies an animal such as a mouse may be administered (e.g., immunized) with one or more HIV proteins. Animals exhibiting serum reactivity to HIV expressed on activated human T lymphocytes (as determined by, for instance, flow cytometry and/or microscopy) may then be selected for generation of anti-HIV hybridoma cell lines. This may be repeated for multiple rounds. Screening may also include, for instance, affinity binding and/or functional characterization to identify the binding agent as being specific for HIV. In some embodiments, such as in the Examples herein, human beings may be screened for the expression of antibodies against HIV. In some embodiments, plasma samples of human beings infected by HIV may be screened to identify persons expressing anti-HIV antibodies, and in particular, neutralizing antibodies. Neutralizing antibody-producing cells of such persons may then be isolated, followed by the isolation and characterization of the antibodies produced thereby (e.g., as in the examples herein). A neutralizing antibody may be one that exhibits the ability to neutralize, or inhibit, infection of cells by HIV. In general, a neutralization assay typically measures the loss of infectivity of the virus through reaction of the virus with specific antibodies. Typically, a loss of infectivity is caused by interference by the bound antibody with any of the virus replication steps including but not limited to binding to target cells, entry, and/or viral release. The presence of un-neutralized virus is detected after a predetermined amount of time, e.g., one, two, three, four, five, six, seven, eight, nine, 10, 12 or 14 days, by measuring the infection of target cells using any of the systems available to those of ordinary skill in the art (e.g., a luciferase-based system). A non-limiting example of a neutralization assay may include combining a given amount of a virus or pseudovirus (see below) and different concentrations of the test or control (typically positive and negative controls assayed separately) antibody or antibodies are mixed under appropriate conditions (e.g., one (1) hour at room temperature) and then inoculated into an appropriate target cell culture (e.g., TZM-bl cells). For instance, binding agent-producing cells (e.g., B cells producing antibodies) may be assayed for the production of HIV-1 neutralizing antibodies by seeding such cells in separate plates as single cell micro-cultures on human feeder cells in the presence of Epstein-Barr Virus (EBV) (which also stimulate polyclonally memory B cells), a cocktail of growth factors (e.g., TLR9 agonist CpG-2006, IL-2 (1000 IU/ml), IL-6 (10 ng/ml), IL-21 (10 ng/ml), and anti-B cell receptor (BCR) goat antibodies (which trigger BCRs). After an appropriate time (e.g., 14 days), supernatants of such cultures may tested in a primary luciferase-based screening system using two or more representative HIV-1 viruses or pseudoviruses that productively infect such cells. The pseudoviruses may be incubated with B cell culture supernatants for an appropriate time and temperature (e.g., one (1) h at 37% (5% CO2)) before the addition of host cells (e.g., 3000 TZM-bl cells). Incubation for an appropriate time (e.g., 72 hours) may then follow, after which the supernatant may be removed and Steadylite reagent (Perkin Elmer) added (e.g., 15 μl). Luciferase activity may then determined (e.g., five minutes later) on a Synergy microplate luminometer (BioTek). Decreased luciferase activity relative to a negative control typically indicates virus neutralization. Neutralization assays such as these, suitable for analyzing binding agents of this disclosure, are known in the art (see, e.g., Montefiori, D. C. Curr. Protocol. Immunol. Chapter 12, Unit 12.11 (2005); Edmonds, et al. Virology, 408(1): 1-13 (2010); Seaman, et al. J. Virol. 84(3): 1439-1452 (2010); Pace, et al. PNAS USA, 110(33): 13540-13545 (2013)). In some embodiments, test samples may be screened for the presence of antibodies able to neutralize a panel of HIV pseudoviruses (e.g., eight (8) HIV-1 pseudoviruses from the Global Panel of HIV-1 reference strains as conducted in the examples herein (those pseudoviruses being TRO.11 (B), 246F3 (AC), BJOX2000 (CRF007_BC), CE1176 (C), CH119 (CRF07_BC), CNE55 (CRF01_AE), 25710 (C), and X1632 (see, e.g., FIGS. 2-5) but not of the control virus SVA-MLV at about 10 μg/ml or less (e.g., FIG. 5); DeCamp, A. et al. Global panel of HIV-1 Env reference strains for standardized assessments of vaccine-elicited neutralizing antibodies. J Virol 88, 2489-2507 (2014)). Neutralization of a larger panel of psuedoviruses may also be tested; for instance, de Camp et al. describe a group of 12 pseudoviruses (also known as HIV-1 Env Reference Strains): 398F1, 25710, CNE8, TRO11, X2278, BJOX2000, X1632, CE1176, 246F3, CH119, CE0217, and CNE55. In some embodiments, a panel of ten HIV isolates may be tested and a bNab may be identified as one that neutralizes six, seven, eight, nine members of a panel of nine pseudoviruses; or six, seven, eight, nine, 10, 11 or 12 members of a panel of 12 pseudoviruses. Screening of larger panels of such pseudoviruses (e.g., a panel of 57 pseudoviruses as in the examples herein) may also be carried out. In one embodiment, then, an exemplary panel of 57 pseudoviruses used in the examples against which test samples may be tested for neutralizing antibodies may include, for instance, those shown in FIGS. 2-5 (e.g., Glade A (T/F), Glade B, Glade B (T/F), Glade BC, Glade C, Glade C (T/F), Glade E (T/F) or Glade G). In some embodiments, neutralization may be determined as a measure of the concentration (e.g., μg/ml) of monoclonal antibody capable of neutralizing any of about 50%, 60%, 70%, 80%, 90%, 95%, or 99% of viral infection (as may be measured by percent neutralization and/or by determining an “IC₅₀” and/or “IC₈₀” value). In some embodiments, a binding agent may be considered neutralizing if it is able to neutralize 50% of viral infection at a concentration of, for instance, about any of 10⁻⁵, 10⁻⁴, 10⁻³, 10⁻², 10¹, 10°, 10¹, 10², or 10³ μg/ml (e.g., an IC₈₀ value as shown in in FIGS. 2-5). In some embodiments, the ability of a neutralizing antibody to neutralize viral infection may be expressed as a percent neutralization (e.g., 10%, 20%, 30%, 40%, 50%, 60%, 70%, 80%, 90%, 95%, or 99% (e.g., as in FIG. 5)). And in some embodiments, as in the Examples herein, the ability of a neutralizing antibody to neutralize viral infection may be expressed as, and, in preferred embodiments, the IC₅₀ and/or IC₈₀ value is below 25 μg/ml, and is even more preferably below about any of 15, 10, 5, 2, 1, 0.5, 0.25, 0.1, 0.05, or 0.01 μg/ml (see, e.g., FIGS. 2-5). Other measures of neutralization may also be suitable as may be determined by those of ordinary skill in the art.

In some embodiments, the binding agents described herein may be broadly neutralizing antibodies (bNabs) identified in biological samples (e.g., plasma) obtained from HIV-infected persons. As mentioned above and shown in the examples herein, such bNabs may be identified by testing plasma samples of patients chronically infected by HIV (preferably those naïve to antiretroviral therapy) for the ability to neutralize multi-Glade HIV isolates (e.g., initially using a nine or 12-member panel and then a larger panel (e.g., 57 members) of pseudoviruses)). In some embodiments, the samples may be derived from patients known to be “Elite Controllers” with viremia <50 HIV RNA copies per ml of plasma. Screening procedures such as these may result in the identification of patients that may serve as lymph node donors for the subsequent isolation and characterization of B cells producing bNabs. In carrying out such screening assays, neutralizing activity is typically compared to a negative control such as murine leukemia virus (MLV) pseudovirus.

In some embodiments, germinal center and memory IgG B cells of patients expressing neutralizing binding agents (e.g., antibodies) may be isolated and further studied. In some embodiments, the cells may be sorted separately according to IgG (e.g., IgA and IgM negative cells), CD19, and CD38 expression (germinal center B cells are CD38 positive) and interrogated for the production of HIV-1 neutralizing antibodies. For instance, highly pure IgG memory B cells and IgG germinal cells may be seeded in separate plates as single cell micro-cultures on human feeder cells in the presence of Epstein-Barr Virus (EBV) (which also stimulate polyclonally memory B cells) and a cocktail of growth factors and the like (e.g., composed TLR9 agonist CpG-2006, IL-2 (1000 IU/ml), IL-6 (10 ng/ml), IL-21 (10 ng/ml), and anti-BCR goat antibodies (B cell receptor (BCR) triggering)). Supernatants of such cultures (e.g., from day 14 cultures) may then be tested in a primary screening (e.g., using a 384-well based HIV-1 pseudoviruses neutralization assay using in parallel two strains, CE1176 and BJOX2000, representative of Glade C and CRF07, as shown in the examples herein). Neutralization assays may be carried out using any suitable host cells (e.g., TZM-bl cells (Seaman, et al. J. Virol. 84(3): 1439-52 (2010); NIH AIDS Reagent Program Catalog Number 8129)). HIV-1 pseudoviruses resulting in a significant output relative light units (RLU) (e.g., of 50-100×10⁴ RLU) (i.e., indicating productive infection of cells) may then incubated with B cell culture supernatants for an appropriate time and temperature (e.g., one (1) h at 37% (5% CO2)) before the addition of host cells (e.g., 3000 TZM-bl cells). Incubation for an appropriate time (e.g., 72 hours) typically follows, after which the supernatant may be removed and Steadylite reagent (Perkin Elmer) added (e.g., 15 μl). Luciferase activity may then be detected (e.g., five minutes later) on a Synergy microplate luminometer (BioTek). Decreased luciferase activity indicates a lesser amount of virus being released by the cells and virus neutralization. For instance, if the base RLU for a particular pseudovirus is 50-100×10⁴ RLU, a neutralizing antibody may be determined to decrease the RLU for that pseudovirus to 25-50×10⁴ RLU (i.e., a 50% decrease), or less. Using such systems, supernatants capable of cross-neutralizing strains may be identified, further harvested, and tested for their ability to neutralize other pseudoviruses.

The antibodies derived from such neutralizing antibody-containing cultures may then be further characterized by determining the amino acid and nucleotide sequences of the antibody variable and complementarity determining regions (CDRs) regions. Using these techniques, the HIV-neutralizing binding agent termed “LN02” was identified as an IgG3-type fully human monoclonal antibody having the CDR, VH and VL sequences shown in FIG. 1 (comprising SEQ ID NOS. 234, 235). As described herein, variants of LN02 have now been developed and shown to exhibit surprising functional properties (e.g., increased HIV pseudovirus neutralization). In some embodiments, the variable heavy chain (V_{H}) and variable light chain (VI) genes of a binding agent may then be cloned into an IgG expression vector of the same or a different isotype. As shown in the examples, for instance, nucleic acids encoding LN02M variable region and/or CDR and/or non-CDR amino acid sequences were cloned into IgG1 backbone, and the recombinant IgG1-based antibody was produced by transfecting appropriate host cells (e.g., Expi293F cells). The antibody full-length IgG1-based antibody may then be purified using standard techniques (e.g., a full-length IgG1-based antibody may be purified using a recombinant protein-A column (GE-Healthcare)). The recombinantly-produced IgG1 antibody may then be tested against any of a panel of pseudoviruses such as any of those described herein (e.g., the Global Panel of nine (9) HIV-1 reference pseudoviruses used in the examples) on an appropriate host cell (e.g., TZM-bl cells). In preferred embodiments, the binding agent will exhibit the ability to neutralize a majority (i.e., at least about 50% or greater) of the pseudovirus panel members (e.g., comprising nine, 12 or 118 members) without neutralizing a negative control virus (e.g., MLV pseudovirus). It is preferred that the binding agent exhibit the ability to neutralize a majority of such viruses (e.g., neutralization of greater than about 50%, such as any of about 60%, 70%, 80%, 90%, 95%, 99%, or 100%) with IC₅₀ and/or IC₈₀ values considered neutralizing (see below). For example, in some embodiments, a binding agent of this disclosure may exhibit neutralization of HIV-1 pseudoviruses TRO.11 (Clade B), 25710 (Clade C), CE1176, BJOX (CRF07_BC), CH119 (CRF07_BC), 246-F3 (Clade AC), X1632 (Clade G), CNE55 (CRF01_AE), and/or CD0217 (Clade C). In some embodiments, neutralization of the HIV-1 pseudoviruses viruses may be observed where the antibody concentration is from 10⁻²−10° μg/ml (i.e., 10 ng/ml to 1 μg/ml), or between 10⁰-10¹ μg/ml (i.e., 1-10 μg/ml). In some such embodiments, the percent neutralization by the binding agent is at least about 50%. In some embodiments, infection of one HIV-1 isolate is considered neutralized by a binding agent (e.g., antibody) at an IC₅₀ and/or IC₈₀ of less than 25 μg/ml, if infection of at least one isolate of this isolate is neutralized with an IC₅₀ of less than 25 μg/ml. In some embodiments, the binding agent may be considered neutralizing where HIV-1 pseudoviruses listed in are considered neutralized at an IC₅₀ of less than 25 μg/ml, such as about 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml, 0.1 μg/ml, 0.09 μg/ml, 0.08 μg/ml, 0.07 μg/ml, 0.06 μg/ml, 0.05 μg/ml, 0.04 μg/ml, 0.03 μg/ml, 0.02 μg/ml, or 0.01 μg/ml; and/or an IC₅₀ of between about 0.001 to about 10 μg/ml. In preferred embodiments, the binding agent may neutralize HIV-1 pseudovirus strains TRO.11 (Clade B), 25710 (Clade C), CE1176, BJOX (CRF07_BC), CH119 (CRF07_BC), 246-F3 (Clade AC), X1632 (Clade G), CNE55 (CRF01_AE), and/or CD0217 (Clade C); and/or, of ID MS208.A1, Q23.17, Q769.d22, Q842.d12, Q259.d2.17, 0260.v5.c36, 191955_A11, 191084 B7-19, TRO.11, 6535.3, REJ04541.67, SC422661.8, QH0692.42, TRJ04551.58, RHPA4259.7, PVO.4, SCO5 8C11 2344, CNE17, CNE19, CNE20, CNE21, Du422.1, CAP210.2.00.E8, ZM249M.PB6, HIV-001428-2.42, ZM214M.PL15, CAP45.2.00.G3, Ce704809221_163, Ce 1 1 76_A3, ZM247v1(Rev-), Ce0682_E4, 249M B10, 246F Cl G, and/or BF1266.431a; at, for example an IC₅₀ or IC₈₀ of less than or about 1 μg/ml (FIGS. 2-4, 9, Tables 7A, 7B, and/or 8A through 8M). It is further preferred that the binding agent not exhibit Glade-dependency. For instance, in some embodiments, the binding agent may exhibit the ability to neutralize pseudoviruses of HIV-1 Clades including but not limited to Glade A (T/F), Glade B, Glade B (T/F), Glade BC, Glade C, Glade C (T/F), Glade E (T/F), and/or Glade G. In some preferred embodiments, the binding agent may neutralize at least one pseudovirus in each of clades A, A(T/F), B, B (T/F), BC, C, C (T/F), and G at an IC₅₀ or IC₈₀ of less than or about 1 μg/ml. In some preferred embodiments, the binding agent may neutralize at least one pseudovirus in each of clades A, A(T/F), B, B (T/F), BC, C, C (T/F) at an IC₅₀ or IC₈₀ of less than or about 0.5 μg/ml. In some preferred embodiments, the binding agent may neutralize at least one pseudovirus in each of clades A, A(T/F), B, B (T/F), BC, C, and C (T/F) at an IC₈₀ of less than or about 1 μg/ml. In some embodiments, the binding agent comprises any one or more of these properties and one or more of the LN02M variable region and/or CDR and/or non-CDR amino acid sequences.

In some embodiments, the binding agents may be tested for neutralization capacity against HIV reference pseudoviruses (e.g., the above-described Global Panel of nine (9) HIV-1 reference pseudoviruses) using cells expressing or not expressing one or more types of Fc receptors (e.g., parental TZM-bl cells and TZM-bl cells expressing Fc-gamma receptor I (CD64) as in the examples; see e.g. Perez, et al. Utilization of immunoglobulin G Fc receptors by human immunodeficiency virus type 1: a specific role for antibodies against the membrane-proximal external region of gp41. J Virol 83, 7397-7410 (2009); NIH AIDS Reagent Program Catalog No. 11798). Enhanced neutralizing activity in cells expressing Fc receptors may provide antibodies a kinetic advantage for virus inhibition. This kinetic advantage could be unique to antibodies, whose epitopes are thought to be difficult to access or exposed for only a short time on intermediate conformations of the Env protein during an early stage of fusion. Fc-gamma receptors could also potentially facilitate HIV-1 neutralization is phagocytosis, thereby increasing neutralization capacity of the antibodies. To this point, HeLa cells, from which the TZM-bl cell line was constructed, are known to exhibit properties of nonprofessional phagocytes. Thus, it is possible that TZM-bl cells were converted to professional phagocytic cells by introducing Fc-gamma receptor on their surface. Any Fc-gamma-receptor-mediated antiviral effects on HIV-1 neutralizing antibodies, whether by entry inhibition or phagocytosis, might be beneficial in HIV treatment and vaccine regimens. Fc-gamma receptors are rarely expressed on CD4+ lymphocytes, but several other HIV-1-susceptible cell types express multiple Fc-gamma receptors and are involved in sexual transmission and the early establishment of long-lived viral reservoirs. In particular, macrophages are among the first infection-susceptible cells that the virus encounters after mucosal exposure, and are thought to serve as a long-lived virus reservoir in chronic infection. Macrophages, as well as certain subsets of monocytes and dendritic cells, are known to express multiple Fc-gamma receptors. It is also important to mention that Fc-gamma receptors play a role in regulating adaptive immunity and peripheral tolerance, by facilitating antigen uptake, antigen presentation, cell activation and B cell tolerance. Thus, is some embodiments, the binding agents described herein may be used in conjunction with agents that induce and/or enhance Fc receptor expression, including the introduction of nucleic acids encoding one or more Fc receptors with or in conjunction with treatment by the binding agents described herein.

The specificity of the binding agents described herein may be determined using any of the many techniques available to those of ordinary skill in the art. For instance, as shown in the examples herein, the specificity of a binding agent (e.g., IgG1 LN02 antibody), with respect to particular epitopes, may be ascertained using a panel of pseudoviruses (e.g., CAP45) that encode mutations in the HIV envelope gene. For instance, modifications can be made to HIV envelope protein (Env) to produce modified Env proteins (mEnv) and each binding agent (e.g., antibody) tested for its ability to bind to the various mEnv proteins. An exemplary HIV-1 envelope amino acid sequence that can be used is that of the CAP45 pseudovirus (GenBank Accession No μF203962; NCBI GenPept Accession No.

ABQ02701.1; SEQ ID NO.: 237) and/or the HXB2 Env sequence (SEQ ID NO.: 238 (GenBank Accession No. MF944225.1, protein_id=ATG88205.1)) as shown below (with exemplary amino acids that could be modified indicated in bold and underlined):

| (CAP45; SEQ ID NO. 237) | |
|---|---|
| 1 | MGVRGILGNW PQWWIWSILG FWMLIICRVM GNLWVTVYYG VPVWKEAKAT LFCASDARAY |
| 61 | EKEVHNVWAT HACVPTDPNP QEIYLG [symbol] VTE NFNMWKNDMV DQMHEDIISL WDQSLKPCVK |
| 121 | LTPLCVTLRC TNATINGSLT EEVKNCSFNI TTELRDKKQK AYALFYRPDV VPLNKNSPSG |
| 181 | NSSEYILINC NTSTITQACP KVSFDPIPIH YCAPAGYAIL KCNNKTFNGT GPCNNVSTVQ |
| 241 | CTHGIKPVVS TQLLLNGSLA EEDIIIKSEN LTNNIKTIIV HLNKSVEIVC RRPNNNTRKS |
| 301 | IRIGPGQAFY ATNDIIGDIR QAHCNINNST WNRTLEQIKK KLREHFLNRT IEFESPSGGD |
| 361 | LEVTTHSFNC GGEFFYCNTT RLFKWSSNVT NDTITIPCRI KQFINMWQGV GRAMYAPPIE |
| 421 | GNITCNSSIT GLLLTRDGGK TDRNDTEIFR PGGGNMKDNW RNELYKYKVV EIKPLGVAPT |
| 481 | [symbol] RR [symbol] V [symbol] [symbol] R [symbol] G [symbol] [symbol] GFLGAAGST MGAASITLTV QARQLLSGIV QQQSNLLRAI |
| 541 | EAQQHMLQLT VWGIKQLQTR VLAIERYLKD QQLLGLWGCS GKLICTTNVP WNSSW [symbol] NKS [symbol] |
| 601 | TDIWD [symbol] MTWI QWDREISNYS NTIYKLLEDS QNQQEQNEKD LLALDSWNNL WNWFNIT [symbol] WL |
| 661 | WYIKIFIMII GGLIGLRIIL GVLSIVKRVR QGYSPLSFQT LTPNPRGLDR LGRIEEEGGE |
| 721 | QDKDRSIRLV NGFLALAWED LRSLCLFSYH RLRDFILIAV RAVELLGSSS LRGLQRGWEA |
| 801 | LKYLGSLLQY WGLELKKSAI NLLDTVAIAV AEGTDRIIEL IQRICRAIRN IPRRIRQGFE |
| 861 | AALL |
| (HXB2; SEQ ID NO. 238) | |
| 1 | MRVKEKYQHL WRWGWRWGTM LLGMLMICSA TEKLWVTVYY GVPVWKEATT TLFCASDAKA |
| 61 | YDTEVHNVWA THACVPTDPN PQEVVLV [symbol] VT ENFNMWKNDM VEQMHEDIIS LWDQSLKPCV |
| 121 | KLTPLCVSLK CTDLKNDTNT NSSSGRMIME KGEIKNCSFN ISTSIRGKVQ KEYAFFYKLD |
| 181 | IIPIDNDTTS YTLTSCNTSV ITQACPKVSF EPIPIHYCAP AGFAILKCNN KTFNGTGPCT |
| 241 | NVSTVQCTHG IRPVVSTQLL LNGSLAEEEV VIRSVNFTDN AKTIIVQLNT SVEINCTRPN |
| 301 | NNTRKKIRIQ RGPGRAFVTI GKIGNMRQAH CNISRAKWNA TLKQIASKLR EQFGNNKTII |
| 361 | FKQSSGGDPE IVTHSFNCGG EFFYCNSTQL FNSTWFNSTW STEGSNNTEG SDTITLPCRI |
| 421 | KQFINMWQEV GKAMYAPPIS GQIRCSSNIT GLLLTRDGGN NNNGSEIFRP GGGDMRDNWR |
| 481 | SELYKYKVVK IEPLGVAPT [symbol] [symbol] KRR [symbol] V [symbol] R [symbol] G [symbol] GFLGAAGSTM GAASMTLTVQ |
| 541 | ARQLLSGIVQ QQNNLLRAIE AQQHLLQLTV WGIKQLQARI LAVERYLKDQ QLLGIWGCSG |
| 601 | KLICTTAVPW NASW [symbol] NKS [symbol] E QIWN [symbol] TTWME WDREINNYTS LIHSLIEESQ NQQEKNEQEL |
| 661 | LELDKWASLW NWFNIT [symbol] WLW YIKLFIMIVG GLVGLRIVFA VLSVVNRVRQ GYSPLSFQTH |
| 721 | LPIPRGPDRP EGIEEEGGER DRDRSIRLVN GSLALIWDDL RSLCLFSYHR LRDLLLIVTR |
| 781 | IVELLGRRGW EALKYWWNLL QYWSQELKNS AVSLLNATAI AVAEGTDRVI EVVQGACRAI |
| 841 | RHIPRRIRQG LERILL |

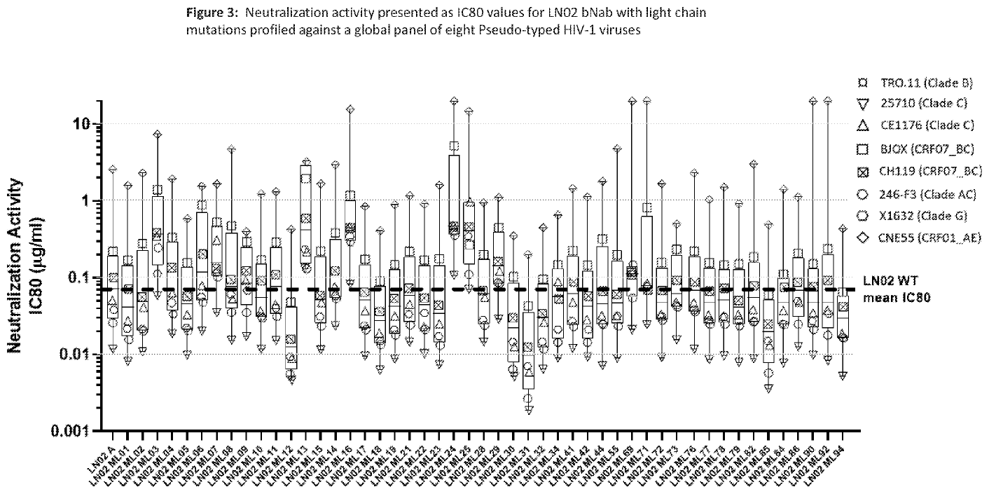

In some embodiments, a binding agent of this disclosure may comprise these binding specificities along with the neutralization characteristics described above (i.e., neutralization of HIV-1 pseudoviruses TRO.11 (Clade B), 25710 (Clade C), CE1176, BJOX (CRF07_BC), CH119 (CRF07_BC), 246-F3 (Clade AC), X1632 (Clade G), CNE55 (CRF01_AE), CD0217(Clade C) at a concentration is from 10⁻² to 10° μg/ml (i.e., 10 ng/ml to 1 μg/ml), or between 10⁰-10¹ μg/ml (i.e., 1-10 μg/ml), to at least about 50% (FIGS. 3, 4, 9; Tables 7A, 7B, and 8A through 8M), as well as the neutralization of a majority of HIV-1 pseudoviruses at an IC₅₀ or IC₈₀ of less than 25 μg/ml.

The specificity of a binding agent may also be tested for binding to soluble trimers representing HIV proteins (e.g., soluble, cleaved SOSIP.664 gp140 trimers based on the subtype A transmitted/founder strain, BG505 as used in the examples herein). Preferred trimers (such as those used in the examples herein) are those being highly stable, homogenous and closely resembling native virus spikes when visualized by negative stain electron microscopy (EM) (Sanders, R. W. et al. A next-generation cleaved, soluble HIV-1 Env trimer, BG505 SOSIP.664 gp140, expresses multiple epitopes for broadly neutralizing but not non-neutralizing antibodies. PLoS Pathog. 9, e1003618 (2013)). Typically, broadly neutralizing antibodies against multiple neutralizing epitopes on HIV-1 Env will be highly reactive with such trimers. Conversely, non-neutralizing antibodies (NAbs) to the CD4-binding site, CD4-induced epitopes or gp41 ectodomain would not (and did not in the example) react with the trimers, even when their epitopes were present on simpler forms of Env (e.g., gp120 monomers or dissociated gp41 subunits). The examples also included a test, which may be used in testing any of binding agents described herein, in which the MPER was also deleted to improve trimer solubility and reduce aggregate formation. The binding agents may also be tested for binding to such trimers in the presence or absence of soluble CD4 (sCD4). The examples herein describe the testing of the LN02 and PGT151 (binding to a site at the interface between gp120 and gp41 (Dingens et al. Cell Host Microbe. 2017 Jun. 14; 21(6):777-787.e4. doi: 10.1016/j.chom.2017.05.003. Epub 2017 Jun. 1)) antibodies for binding to the 426c WT SOSIP Env protein complex, measured by surface plasmon resonance (SPR)). As shown in the examples, the biotinylated IgG1 LN02 antibody was shown to bind the 426c WT SOSIP Env protein but was completely blocked from binding the Env protein that had been pre-incubated with an unlabeled LN02 antibody. In a similar experiment, the biotinylated interface binding bNab PGT151 bound tightly to 426c WT SOSIP Env protein but PGT151 bound more weakly to the LN02+426c WT SOSIP Env protein complex. The results presented in the examples therefore indicate that IgG1 LN02 antibody might recognize an epitope at the gp120/gp41 interface of HIV-1 Env. Given that PGT151 binding to 426c WT SOSIP was not completely blocked by LN02, it is possible that LN02 binds in the same region but not identical epitope compared to PGT151. Other assay systems including surface plasmon resonance may be used to test the binding agents contemplated herein. And similar tests may also be performed on any of the binding agents contemplated herein.

The term “binding affinity” and/or K_{D} refers to the dissociation rate of a particular antibody-antigen interaction. The K_{D} is the ratio of the rate of dissociation (“off-rate (k_{d})”) to the association rate (“on-rate (kₐ)). K_{D} therefore equals k_{d}/kₐ and is expressed as a molar concentration (M). Thus, the smaller the K_{D}, the stronger the affinity of binding. For example, a K_{D} of 1 mM indicates weak binding as compared to a K_{D} of 1 nM. K_{D} values for antibodies can be determined using methods well established in the art such as by using a Biacore® system. In some embodiments, the binding agents described herein may be compared with another binding agent with reference to the respective K_{D} values of each. These properties may be combined with other characteristics such as neutralization capacity and/or epitope specificity in order to compare binding agents to one another. Accordingly, binding agents having a similar K_{D} to those described herein, perhaps also sharing the neutralization capacity and epitope specificity described herein (e.g., as exhibited by LN02M), are also contemplated as part of this disclosure.

Any of the amino acid sequences of LN02M variable region and/or CDR and/or non-CDR amino acid sequences (and/or any one or more fragments and/or derivatives thereof) may be also substituted by any other amino acid as desired by one of ordinary skill in the art. For example, one of skill in the art may make conservative substitutions by replacing particular amino acids with others as shown in Table 5 below. The specific amino acid substitution selected may depend on the location of the site selected. An amino acid substitution may be said to “correspond to” where one of ordinary skill in the art could ascertain a significant amount of similarity between the amino acid sequences surrounding the amino acid being substituted. For instance, a particular amino acid sequence may correspond to another where two, three, four or more N-terminal and C-terminal amino acids surrounding the amino acid being substituted are the same or similar (e.g., as described in Table 5) in the polypeptides being compared. Conservative amino acid substitutions may involve a substitution of a native amino acid residue with a non-native residue such that there is little or no effect on the size, polarity, charge, hydrophobicity, or hydrophilicity of the amino acid residue at that position and, in particular, does not result in, e.g., decreased HIV neutralization capacity and/or different epitope specificity.

TABLE 5

| | | Preferred |
|---|---|---|
| Original | | Conservative |
| Amino | | Substitution of the |
| Acid | Exemplary Conservative Substitutions | Original Amino |
| Residue | of the Original Amino Acid Residue | Acid Residue |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

In certain embodiments, a nucleic acid molecule encoding one or more binding agents described herein may be inserted into one or more expression vectors, as discussed below in greater detail. In such embodiments, the binding agent may be encoded by nucleotides corresponding to the amino acid sequence. The particular combinations of nucleotides (codons) that encode the various amino acids (AA) are well known in the art, as described in various references used by those skilled in the art (e.g., Lewin, B. Genes V, Oxford University Press, 1994). The nucleotide sequences encoding the amino acids of said binding agents may be ascertained with reference to Table 6, for example. Nucleic acid variants may use any combination of nucleotides that encode the binding agent.

TABLE 6
Codons Encoding Amino Acids (AA)

| AA | Codon |
|---|---|
| Phe (F) | TTT |
| | TTC |
| Leu (L) | TTA |
| | TTG |
| | CTT |
| | CTC |
| | CTA |
| | CTG |
| Ile (I) | ATT |
| | ATC |
| | ATA |
| Met (M) | ATG |
| Val (V) | GTT |
| | GTC |
| | GTA |
| | GTG |
| Ser (S) | TCT |
| | TCC |
| | TCA |
| | TCG |
| Pro (P) | CCT |
| | CCC |
| | CCA |
| | CCG |
| Thr (T) | ACT |
| | ACC |
| | ACA |
| | ACG |
| Ala (A) | GCT |
| | GCC |
| | GCA |
| | GCG |
| Tyr (Y) | TAT |
| | TAC |
| TERM | TAA |
| | TAG |
| His (H) | CAT |
| | CAC |
| Gln (Q) | CAA |
| | CAG |
| Asn (N) | AAT |
| | AAC |
| Lys (K) | AAA |
| | AAG |
| Asp (D) | GAT |
| | GAC |
| Glu (E) | GAA |
| | GAG |
| Cys (C) | TGT |
| | TGC |
| TERM | TGA |
| Trp (W) | TGG |
| Arg (R) | CGT |
| | CGC |
| | CGA |
| | CGG |
| Ser (S) | AGT |
| | AGC |
| Arg (R) | AGA |
| | AGG |
| Gly (G) | GGT |
| | GGC |
| | GGA |
| | GGG |

Those of ordinary skill in the art understand that the nucleotide sequence encoding a particular amino acid sequence may be easily derived from the amino acid sequence of any of LN02M variable region and/or CDR and/or non-CDR amino acid sequences and the information presented in Table 6. For instance, it may be deduced from the amino acid sequence YGSISRHFWG (SEQ ID NO.: 1) and the information presented in Table 6 that the amino acid sequence may be encoded by the nucleotide sequence TATGGCAGCATTAGCCGCCATTTTTGGGGC (SEQ ID NO.: 34). Those of ordinary skill in the art would understand that nucleotide sequences encoding LN02M variable region and/or CDR and/or non-CDR amino acid sequences may be deduced in the same way, and such nucleotide sequences are contemplated herein. Expression vectors comprising such nucleic acid sequences are also contemplated by this disclosure. Where the binding agents are antibodies, nucleotide sequences encoding the variable regions thereof may also be isolated from the phage and/or hybridoma cells expressing the same cloned into expression vectors. Methods for producing such preparations are well-known in the art.

Nucleic acid molecules encoding one or more HIV binding agents may be contained within a viral and/or a non-viral vector. In one embodiment, a DNA vector is utilized to deliver nucleic acids encoding one or more HIV binding agents to the patient. In doing so, various strategies may be utilized to improve the efficiency of such mechanisms including, for example, the use of self-replicating viral replicons (Caley, et al. 1999. Vaccine, 17: 3124-2135; Dubensky, et al. 2000. Mol. Med. 6: 723-732; Leitner, et al. 2000. Cancer Res. 60: 51-55), codon optimization (Liu, et al. 2000. Mol. Ther., 1: 497-500; Dubensky, supra; Huang, et al. 2001. J. Virol. 75: 4947-4951), in vivo electroporation (Widera, et al. 2000. J. Immunol. 164: 4635-3640), incorporation of nucleic acids encoding co-stimulatory molecules, cytokines and/or chemokines (Xiang, et al. 1995. Immunity, 2: 129-135; Kim, et al. 1998. Eur. J. Immunol., 28: 1089-1103; Iwasaki, et al. 1997. J. Immunol. 158: 4591-3301; Sheerlinck, et al. 2001. Vaccine, 19: 2647-2656), incorporation of stimulatory motifs such as CpG (Gurunathan, supra; Leitner, supra), sequences for targeting of the endocytic or ubiquitin-processing pathways (Thomson, et al. 1998. J. Virol. 72: 2246-2252; Velders, et al. 2001. J. Immunol. 166: 5366-5373), prime-boost regimens (Gurunathan, supra; Sullivan, et al. 2000. Nature, 408: 605-609; Hanke, et al. 1998. Vaccine, 16: 439-445; Amara, et al. 2001. Science, 292: 69-74), proteasome-sensitive cleavage sites, and the use of mucosal delivery vectors such as Salmonella (Darji, et al. 1997. Cell, 91: 765-775; Woo, et al. 2001. Vaccine, 19: 2945-2954). Other methods are known in the art, some of which are described below. Various viral vectors that have been successfully utilized for introducing a nucleic acid to a host include retrovirus, adenovirus, adeno-associated virus (AAV), herpes virus, and poxvirus, among others. The vectors may be constructed using standard recombinant techniques widely available to one skilled in the art. Such techniques may be found in common molecular biology references such as Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), and PCR Protocols: A Guide to Methods and Applications (Innis, et al. 1990. Academic Press, San Diego, ca). “Non-viral” plasmid vectors may also be suitable in certain embodiments. Preferred plasmid vectors are compatible with bacterial, insect, and/or mammalian host cells. Such vectors include, for example, PCR-ii, PCR3, and pcDNA3.1 (Invitrogen, San Diego, Calif.), pBSii (Stratagene, La Jolla, Calif.), pet15 (Novagen, Madison, Wis.), pGEX (Pharmacia Biotech, Piscataway, N.J.), pEGFp-n2 (Clontech, Palo Alto, Calif.), pETI (Bluebacii, Invitrogen), pDSR-alpha (PCT pub. No. WO 90/14363) and pFASTBACdual (Gibco-BRL, Grand island, NY) as well as Bluescript® plasmid derivatives (a high copy number COLe1-based phagemid, Stratagene Cloning Systems, La Jolla, Calif.), PCR cloning plasmids designed for cloning TAQ-amplified PCR products (e.g., TOPO™ TA cloning® kit, PCR2.1® plasmid derivatives, Invitrogen, Carlsbad, Calif.). Bacterial vectors may also be used. These vectors include, for example, Shigella, Salmonella, Vibrio cholerae, Lactobacillus, Bacille Calmette Guérin (BCG), and Streptococcus (see for example, WO 88/6626; WO 90/0594; WO 91/13157; WO 92/1796; and WO 92/21376). Many other non-viral plasmid expression vectors and systems are known in the art and may be use. Other delivery techniques may also suffice including, for example, DNA-ligand complexes, adenovirus-ligand-DNA complexes, direct injection of DNA, CaPO₄ precipitation, gene gun techniques, electroporation, and colloidal dispersion systems. Colloidal dispersion systems include macromolecule complexes, nanocapsules, microspheres, beads, and lipid-based systems including oil-in-water emulsions, micelles, mixed micelles, and liposomes. The preferred colloidal system is a liposome, which are artificial membrane vesicles useful as delivery vehicles in vitro and in vivo. RNA, DNA and intact virions can be encapsulated within the aqueous interior and be delivered to cells in a biologically active form (Fraley, R., et al., 1981, Trends Biochem. Sci., 6: 77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

A cultured cell comprising the vector is also provided. The cultured cell may be a cultured cell transfected with the vector or a progeny of the cell, wherein the cell expresses the immunogenic polypeptide. Suitable cell lines are known to those of skill in the art and are commercially available, for example, through the American Type Culture Collection (ATCC). The transfected cells can be used in a method of producing an immunogenic polypeptide. The method comprises culturing a cell comprising the vector under conditions that allow expression of the immunogenic polypeptide, optionally under the control of an expression sequence. The immunogenic polypeptide can be isolated from the cell or the culture medium using standard protein purification methods. In some embodiments, the binding agents described herein may be conjugated to active agents to target and inhibit the function of and/or eliminate cell populations expressing HIV polypeptides and/or harboring HIV (and/or another antigen in the case of binding agents with multiple specificities). For instance, CD4⁺ T-cell populations containing replication competent HIV may be targeted and eliminated using binding agent/drug conjugates (e.g., antibody-drug conjugates (ADC)). Mono- and/or bi-specific candidate binding agents may be conjugated with one or more types of drugs (e.g., drugs damaging DNA, targeting microtubules). The binding agents described herein and/or derivatives thereof may also be adjoined to and/or conjugated to functional agents for in vitro and/or in vivo use. For instance, the binding agent may be adjoined to and/or conjugated to functional moieties such as cytotoxic drugs or toxins, and/or active fragments thereof such as diphtheria A chain, exotoxin A chain, ricin A chain, abrin A chain, curcin, crotin, phenomycin, enomycin, among others. Suitable functional moieties may also include radiochemicals. Binding agents, such as antibodies, may be adjoined to and/or conjugated to the one or more functional agents using standard techniques in the art.

In some embodiments, this disclosure provides binding agents with multiple specificities such that epitopes bound by LN02M binding agent and at least one other secondary antigen (e.g., a cell surface protein) may be bound by a single binding agent. In some embodiments, the secondary antigen may be one expressed by cells infected by an infectious agent. For instance, an exemplary secondary antigen may be HIV Env antigen other than gp41. Such binding agents may bind the secondary antigen and/or may serve to neutralize the infectious agent as may be determined using the assays described herein. Combinations of binding agents, such as one or more described herein with another available to those of ordinary skill in the art, are also contemplated herein. For instance, in some embodiments, the combinations may be identified to provide statistically significant differences from results (e.g., neutralization assays) obtained using only one or more of the binding agents and not others. In some embodiments, combinations exhibit additive and/or, preferably synergistic, neutralization of HIV, for example. In some embodiments, the combination may comprise a first binding agent having the characteristics of an LN02M binding agent (i.e., comprising a LN02M variable region and/or CDR and/or non-CDR amino acid sequences), and/or derivatives thereof, and any one or more of the antibodies described in any one or more of U.S. Pat. Nos. 5,087,557; 5,298,419; 5,459,060; 5,693,752; 5,731,189; 5,753,503; 5,756,674; 5,777,074; 5,804,440; 5,831,034; 6,008,044; 7,774,88762; U.S. Pat. Publications 2003/0118985A1, 2007/0292390A1, or 2014/0205612A1; WO 2002/032452A1 (e.g., binding the gp41 epitopes ELDKWA, ELEKWA, ELNKWA, ELDEWA); EP0335134B1 US 176077 (e.g, a humanized version of the mouse mAbs described therein); DE3932461A1 (mAb against the epitope Arg-Ile-Leu-Ala-Val-Glu-Arg-Leu-Lys-Try-Asp-Gln-Gln-Leu-Leu-Gly-Ile-Trp-Gly-Cys-Ser); Evans, et al. J. Immunol. 140(3): 941-3 (1988); Gorney, et al. Proc. Natl. Acad. Sci. USA, 86: 1624-28 (1989); Teeuwsen, et al. (1990) AIDS Res. Hum. Retroviruses 6, 381-392; Earl, et al. J. Virol. 71(4): 2674-2684 (1997); Jiang, et al. J. Virol. 72(12): 10213-17 (1998); Zwick, et al. J. Virol. 75(22): 10892-10905 (2001); Eckert et al. PNAS USA, 98(20): 11187-11192 (2001); Louis, et al. J. Biol. Chem. 278(22): 20278-20285 (2003); and/or Pietzsch, et al. J. Virol. 84(10): 5032-42 (2010); all of which are incorporated herein in their entirety. For instance, any of the binding agents described herein may be combined with (i.e., as a single composition, and/or used in conjunction with) one or more the antibodies commonly known as 2F5, 4E10 and/or Z13e1, and/or derivatives thereof, among others. The binding agents of such compositions may be different entities such as two or more different monoclonal antibodies or derivatives thereof, or may be found on the same entity such as a bi-functional antibody (a single antibody or derivative thereof comprising multiple binding specificities). Such combinations as described herein may also be combined with one or more other agents that may affect immune cell function such as antibodies against CTLA-4, and the like. One of ordinary skill in the art would recognize that many such combinations may be suitable for use as described herein.

As mentioned above, the HIV binding agents described herein may be used to treat and/or prevent and/or ameliorate the symptoms of infection by HIV. As is well-known in the art, HIV isolates are now classified into discrete genetic subtypes. HIV-1 is known to comprise at least ten subtypes (A1, A2, A3, A4, B, C, D, E, F1, F2, G, H, J and K) (Taylor et al, NEJM, 359(18):1965-1966 (2008)). HIV-2 is known to include at least five subtypes (A, B, C, D, and E). Subtype B has been associated with the HIV epidemic in homosexual men and intravenous drug users worldwide. Most HIV-1 immunogens, laboratory adapted isolates, reagents and mapped epitopes belong to subtype B. In sub-Saharan Africa, India and China, areas where the incidence of new HIV infections is high, HIV-1 subtype B accounts for only a small minority of infections, and subtype HIV-1 C appears to be the most common infecting subtype. Any of these types of isolates may be addressed using the binding agents described herein. One or more binding agents may also be administered with or in conjunction with one or more agents used to prevent, treat and/or ameliorate HIV such as for example, a protease inhibitor, an HIV entry inhibitor, a reverse transcriptase inhibitor, and/or an anti-retroviral nucleoside analog. Suitable compounds include, for example, Agenerase (amprenavir), Combivir (Retrovir/Epivir), Crixivan (indinavir), Emtriva (emtricitabine), Epivir (3tc/lamivudine), Epzicom, Fortovase/Invirase (saquinavir), Fuzeon (enfuvirtide), Hivid (ddc/zalcitabine), Kaletra (lopinavir), Lexiva (Fosamprenavir), Norvir (ritonavir), Rescriptor (delavirdine), Retrovir/AZT (zidovudine), Reyatax (atazanavir, BMS-232632), Sustiva (efavirenz), Trizivir (abacavir/zidovudine/lamivudine), Truvada (Emtricitabine/Tenofovir DF), Videx (ddl/didanosine), Videx EC (ddl, didanosine), Viracept (nevirapine), Viread (tenofovir disoproxil fumarate), Zerit (d4T/stavudine), and Ziagen (abacavir) may be utilized. Other suitable agents are known to those of skill in the art and may be suitable for use as described herein. Such agents may either be used prior to, during, or after administration of the binding agents and/or use of the methods described herein.

The skilled artisan has many suitable techniques for using the binding agents (e.g., antibodies) described herein to identify biological samples containing proteins that bind thereto. For instance, antibodies may be utilized to isolate HIV or cells containing HIV and/or expressing HIV antigens using, for example, immunoprecipitation or other capture-type assay. This well-known technique is performed by attaching the antibody to a solid support or chromatographic material (e.g., a bead coated with Protein A, Protein G and/or Protein L). The bound antibody is then introduced into a solution either containing or believed to contain HIV antigens (e.g., an HIV-infected cell). The HIV antigen(s) may then bind to the antibody and non-binding materials are washed away under conditions in which the HIV antigen(s) remains bound to the antibody. The bound protein may then be separated from the antibody and analyzed as desired. Similar methods for isolating a protein using an antibody are well-known in the art. The binding agents (e.g., antibodies) may also be utilized to detect HIV or HIV antigens within a biological sample. For instance, the antibodies may be used in assays such as, for example, flow cytometric analysis, ELISA, immunoblotting (e.g., western blot), in situ detection, immunocytochemistry, and/or immunohistochemistry. Methods of carrying out such assays are well-known in the art. In some embodiments, the binding agents may be adjoined to and/or conjugated to one or more detectable labels. For instance, suitable detectable labels may include, for instance, fluorosceins (e.g., DyLight, Cy3, Cy5, FITC, HiLyte Fluor 555, HiLyte Fluor 647; 5-carboxy-2,7-d ichlorofluorescein; 5-Carboxyfluorescein (5-FAM); 5-HAT (Hydroxy Tryptamine); 5-Hydroxy Tryptamine (HAT); 6-JOE; 6-carboxyfluorescein (6-FAM); FITC; 6-carboxy-1,4-dichloro-2′,7′-dichlorofluorescein (TET); 6-carboxy-1,4-dichloro-2′,4′, 5′, 7′-tetra-chlorofluorescein (HEX); 6-carboxy-4′,5′-dichloro-2′, 7′-dimethoxyfluorescein (JOE); Alexa fluors (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE)), rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethylrhodamine), Tetramethylrhodamine (TRITC), WT), Texas Red, and/or Texas Red-X. Other detectable labels known in the art may also be suitable for use. Binding agents, such as antibodies, may be adjoined to and/or conjugated to the one or more detectable labels using standard techniques in the art.

The binding agents described herein may be also be used to determine the presence of a disease state in a patient, to predict prognosis, or to determine the effectiveness of a chemotherapeutic or other treatment regimen. Expression profile assays, performed as described herein or as is otherwise known in the art, may be used to determine the relative level of expression of HIV in a cell, for instance. The level of expression may then be correlated with base (e.g., control) levels to determine whether a particular disease is present within the patient, the patient's prognosis, or whether a particular treatment regimen is effective. For example, if the patient is being treated with a particular anti-infective regimen, an increased or decreased level of expression of HIV in the patient's tissues (e.g., in plasma) may indicate the regimen is worsening or improving the load of HIV in that host. The increase or decrease in expression may indicate the regimen is having or not having the desired effect and another therapeutic modality may therefore be selected.

It is also possible to use the binding agents described herein as reagents in drug screening assays to test, for example, new drug candidates. The reagents may be used to ascertain the effect of a drug candidate on the expression of the immunogenic target in a cell line, or a cell or tissue of a patient. The expression profiling technique may be combined with high throughput screening techniques to allow rapid identification of useful compounds and monitor the effectiveness of treatment with a drug candidate (see, for example, Zlokarnik, et al., Science 279, 84-8 (1998)). Drug candidates may be chemical compounds, nucleic acids, proteins, antibodies, or derivatives therefrom, whether naturally occurring or synthetically derived. Drug candidates thus identified may be utilized, among other uses, as pharmaceutical compositions for administration to patients or for use in further screening assays.

In some embodiments, the binding agents are in purified form. A “purified” binding agent (e.g., antibody) may be one that is separated from at least about 50% of the proteins and/or other components with which it is initially found (e.g., as part of a hybridoma supernatant or ascites preparation in the case of a monoclonal antibody). A purified binding agent (e.g., antibody) may be one that is separated from at least about 50%, 60%, 75%, 90%, or 95% of the proteins and/or other components with which it is initially found.

The binding agents (e.g., polypeptides, antibodies) and nucleic acids described herein may also be combined with one or more pharmaceutically acceptable carriers prior to administration to a host. A pharmaceutically acceptable carrier is a material that is not biologically or otherwise undesirable, e.g., the material may be administered to a subject, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. Suitable pharmaceutical carriers and their formulations are described in, for example, Remington's: The Science and Practice of Pharmacy, 21St Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally from about 5 to about 8 or from about 7 to about 7.5. Other carriers include sustained-release preparations such as semipermeable matrices of solid hydrophobic polymers containing polypeptides or fragments thereof. Matrices may be in the form of shaped articles, e.g., films, liposomes or microparticles. It will be apparent to those persons skilled in the art that certain carriers may be more preferable depending upon, for instance, the route of administration and concentration of composition being administered. Carriers are those suitable for administration of polypeptides and/or fragments thereof to humans or other subjects. Pharmaceutical compositions may also include carriers, thickeners, diluents, buffers, preservatives, surface active agents, adjuvants, immunostimulants, in addition to the immunogenic polypeptide. Pharmaceutical compositions may also include one or more active ingredients such as antimicrobial agents, anti-inflammatory agents and anesthetics. The pharmaceutical composition may be administered orally, parentally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. A “pharmaceutical composition” is a composition comprising a therapeutically effective amount of a nucleic acid or polypeptide. The terms “effective amount” and “therapeutically effective amount” each refer to the amount of a binding agent, nucleic acid or the like used to observe the desired therapeutic effect (e.g., eliminating HIV).

Methods for treating one or more disease conditions (e.g., HIV or cancer) in a mammalian host comprising administering to the mammal at least one or more effective doses of one or more binding agents (and/or derivative(s) thereof) described herein are also provided. In some embodiments, the binding agent is a monoclonal antibody or fragment or derivative thereof comprising one or more of LN02M variable region and/or CDR and/or non-CDR amino acid sequences (i.e., comprising LN02M variable region and/or CDR and/or non-CDR amino acid sequences). The one or more binding agents may be administered in a dosage amount of about 1 to about 50 mg/kg, about 1 to about 30 mg/kg, or about 5 to about 30 mg/kg (e.g., about any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, or 40 mg/kg). In certain embodiments, the one or more binding agents may be administered to the mammal (e.g., intradermally, intravenously, orally, rectally) at about 10 mg/kg one or more times. When multiple doses are administered, the doses may comprise about the same or different amount of binding agent in each dose. The doses may also be separated in time from one another by the same or different intervals. For instance, the doses may be separated by about any of 6, 12, 24, 36, 48, 60, 72, 84, or 96 hours, one week, two weeks, three weeks, one month, two months, three months, four months, five months, six months, seven months, eight months, nine months, 10 months, 11 months, 12 months, 1.5 years, 2 years, 3 years, 4 years, 5 years, or any time period before, after, and/or between any of these time periods. In some embodiments, the binding agents may be administered in conjunction with other agents (e.g., anti-infective agents and/or chemotherapeutic agent). Such other agents may be administered about simultaneously with the binding agents, or at a different time and/or frequency. Other embodiments of such methods may also be appropriate as could be readily determined by one of ordinary skill in the art.

To assist the skilled artisan in using the binding agents such as antibodies described herein, the same may be provided in kit format. A kit including one or more of such binding agents and optionally other components necessary for using the same to detect cells expressing HIV is also provided. The binding agents of the kit may be provided in any suitable form, including frozen, lyophilized, or in a pharmaceutically acceptable buffer such as TBS or PBS. The kit may also include other reagents required for utilization of the binding agents in vitro or in vivo such as buffers (e.g., TBS, PBS), blocking agents (solutions including nonfat dry milk, normal sera, Tween-20 Detergent, BSA, or casein), and/or detection reagents (e.g., goat anti-mouse IgG biotin, streptavidin-HRP conjugates, allophycocyanin, B-phycoerythrin, R-phycoerythrin, peroxidase, detectable labels, and other labels and/or staining kits (e.g., ABC Staining Kit, Pierce)). The kits may also include other reagents and/or instructions for using the antibodies in commonly utilized assays described above such as, for example, flow cytometric analysis, ELISA, immunoblotting (e.g., western blot), in situ detection, immunocytochemistry, and/or immunohistochemistry. In one embodiment, the kit provides a binding agent in purified form. In another embodiment, the binding agent may be provided in biotinylated form either alone or along with an avidin-conjugated detection reagent (e.g., antibody). In another embodiment, the kit includes a binding agents comprising one or more detectable labels that may be used to directly detect HIV. Buffers and the like required for using any of these systems are well-known in the art and/or may be prepared by the end-user or provided as a component of the kit. The kit may also include a solid support containing positive- and negative-control protein and/or tissue samples. For example, kits for performing spotting or western blot-type assays may include control cell or tissue lysates for use in SDS-PAGE or nylon or other membranes containing pre-fixed control samples with additional space for experimental samples. Kits for visualization of HIV in cells on slides may include pre-formatted slides containing control cell or tissue samples with additional space for experimental samples. Other embodiments of kits are also contemplated herein as would be understood by those of ordinary skill in the art.

Thus, this disclosure provides binding agents such as the LN02 antibody with specificity for HIV (e.g., and/or an antigen thereof). In some embodiments, the binding agent is a polypeptide comprising at least one amino acid sequence selected from the group consisting of one or more LN02M variable region and/or CDR and/or non-CDR amino acid sequences. In some embodiments, the binding agent is a polypeptide comprising one or more combinations of LN02M variable region and/or CDR and/or non-CDR amino acid sequences. In some embodiments, the binding agent is an antibody. In some embodiments, the binding agent is a polypeptide such as an antibody comprising heavy and/or light chain CDRs and/or additional amino acid sequence shown for any of the binding agents (e.g., antibodies or derivatives thereof) of FIG. 1; a variable region shown in FIGS. 6A through 6E; heavy chain mutants of FIGS. 7A through 7D; light chain mutants of FIGS. 8A through 8F; any one or more of SEQ ID NOS. 3-92, 95-233, 248-482, or 491-699; and/or a conservatively substituted variant thereof; and/or a non-conservatively substituted variant thereof as described herein.

In some embodiments, the binding agents have specificity for an epitope comprising amino acid residues in the proximity of the gp120/gp41 interface of HIV-1 Env (corresponding residues underlined in SEQ ID NO. 237). In some embodiments, a binding agent of this disclosure may comprise any one or more of these binding specificities along with the neutralization characteristics described above but not of a control virus at a concentration is from 10²−10° ug/ml, or between 10⁰-10¹ ug/ml, to at least about 50%, and/or the ability to the neutralize HIV-1 pseudoviruses at an IC₅₀ or IC₈₀ of less than 25.

In some embodiments, the binding agent is derived from or related to (e.g., by sequence or derivation) a human antibody, human IgG, human IgG1, human IgG2, human IgG2a, human IgG2b, human IgG3, human IgG4, human IgM, human IgA, human IgA1, human IgA2, human IgD, human IgE, canine antibody, canine IgGA, canine IgGB, canine IgGC, canine IgGD, chicken antibody, chicken IgA, chicken IgD, chicken IgE, chicken IgG, chicken IgM, chicken IgY, goat antibody, goat IgG, mouse antibody, mouse IgG, pig antibody, and/or rat antibody, and/or a derivative thereof. In some embodiments, the derivative may be selected from the group consisting of an F_{ab}, F_{ab2}, Fab′ single chain antibody, Fᵥ, single chain, mono-specific antibody, bispecific antibody, trimeric antibody, multi-specific antibody, multivalent antibody, chimeric antibody, canine-human chimeric antibody, canine-mouse chimeric antibody, antibody comprising a canine Fc, humanized antibody, human antibody, caninized antibody, CDR-grafted antibody, shark antibody, nanobody, and/or camelid antibody. In some embodiments, the binding agent comprises at least a least a first and second specificity, the first being against HIV gp41 and the second being against a different antigen (e.g., an antigen of an infectious agent such as HIV (e.g., env) and/or a tumor antigen). In some embodiments, the binding agent and/or derivative thereof may comprise a detectable label fixably attached thereto. In some embodiments, the binding agent of any one and/or derivative thereof comprises an effector moiety (e.g., a cytotoxic drug, toxin, diphtheria A chain, exotoxin A chain, ricin A chain, abrin A chain, curcin, crotin, phenomycin, enomycin, and radiochemical) fixably attached thereto. In some embodiments, polynucleotides encoding one or more binding agents are also provided (e.g., as an expression vector). Host cells comprising and/or expressing the polypeptide products of such polynucleotides are also provided. In some embodiments, compositions comprising at least one binding agent or derivative; at least one isolated polynucleotide; at least one expression vector; and/or, at least one host cell; or a combination thereof; and, a pharmaceutically acceptable carrier are also provided.

This disclosure also provides methods for detecting HIV on a cell, the method comprising contacting a test biological sample with a binding agent or derivative described herein and detecting the binding agent bound to the biological sample or components thereof. Such methods may be an in vivo method or an in vitro method. In some embodiments, the method may comprise comparing the amount of binding to the test biological sample or components thereof to the amount of binding to a control biological sample or components thereof, wherein increased binding to the test biological sample or components thereof relative to the control biological sample or components thereof indicates the presence of a cell expressing HIV polypeptides in the test biological sample (e.g., mammalian blood). In some embodiments, a kit for detecting the expression of HIV in or on a cell, the kit comprising a binding agent or derivative thereof and instructions for use. In some embodiments, the binding agent and/or derivative thereof is in lyophilized form. In some embodiments, this disclosure provides methods for treating, preventing and/or ameliorating an infectious disease, cancer and/or autoimmunity in a mammal comprising administering to the mammal at least one effective dose of a pharmaceutical composition comprising a binding agent or derivative thereof. In some embodiments, the infectious disease is human immunodeficiency virus (HIV). In some embodiments, multiple doses are administered to the animal. In some embodiments, the binding agent and/or derivative thereof may be administered in a dosage amount of about 1 to 50 mg/kg.

In some embodiments, this disclosure provides a binding agent(s) comprising a variable region shown in FIGS. 6A through 6E; amino acid sequence of any mutant of FIGS. 7A through 7D and/or FIGS. 8A through 8F, and any effective (e.g., HIV neutralization) combination thereof; any one or more of SEQ ID NOS. 3-92, 95-233, 248-482, or 491-699, and any effective (e.g., HIV neutralization) combination thereof; a combination of light and heavy chains shown in Table 9 (i.e., ML085, Mx152, MX067, MX129, MX130, ML126, Mx175, Mx176, and Mx181); a combination of light and heavy chains shown in Tables 10A through 10C, 11, 12A through 12D, 13A through 13D, or 14; as well as variants thereof. This disclosure provides a binding agent (e.g., a polypeptide such as an antibody), or combination thereof, the binding comprising: a) at least one CDR illustrated in FIG. 1 (i.e., the amino acid sequences corresponding to CDR1, CDR2, and/or CDR3 of the LN02 bNab Heavy Chain or LN02 bNab Light Chain shown therein, including one or more, or all, of the amino acid substitutions illustrated therein); b) an amino acid sequence selected from the group consisting of SEQ ID NOS. 3-92 or 491-699, preferably including one or more CDR thereof (the CDRs being underlined in the LN02_light chain amino acid sequence of SEQ ID NO. 1 in FIG. 6D); an amino acid sequence selected from the group consisting of SEQ ID NOS. 95-233 or 248-482, preferably including one or more CDR thereof (the CDRs being underlined in the LN02_HEAVY CHAIN amino acid sequence of SEQ ID NO. 93 in FIG. 6A); a variable heavy chain region comprising MH01 (SEQ ID NO. 95), MH16 (SEQ ID NO. 110), MH22 (SEQ ID NO. 116), MH26 (SEQ ID NO. 120), MH30 (SEQ ID NO. 124), MH32 (SEQ ID NO. 126), MH35 (SEQ ID NO. 129), MH36 (SEQ ID NO. 130), MH37 (SEQ ID NO. 131), MH43 (SEQ ID NO. 136), MH44 (SEQ ID NO. 137), MH48 (SEQ ID NO. 141), MH49 (SEQ ID NO. 142), MH50 (SEQ ID NO. 143), MH51 (SEQ ID NO. 144), MH53 (SEQ ID NO. 146), MH59 (SEQ ID NO. 151), MH61 (SEQ ID NO. 153), MH64 (SEQ ID NO. 156), MH68 (SEQ ID NO. 159), MH73 (SEQ ID NO. 163), MH84 (SEQ ID NO. 174), MH89 (SEQ ID NO. 177), MH91 (SEQ ID NO. 178), MH92 (SEQ ID NO. 179), MH106 (SEQ ID NO. 193), MH107 (SEQ ID NO. 194), MH108 (SEQ ID NO. 195), MH111 (SEQ ID NO. 198), MH112 (SEQ ID NO. 199), MH115 (SEQ ID NO. 202), MH119 (SEQ ID NO. 206), MH120 (SEQ ID NO. 207), MH124 (SEQ ID NO. 211), MH 131 (SEQ ID NO. 218), MH135 (SEQ ID NO. 222), MH136 (SEQ ID NO. 223), MH138 (SEQ ID NO. 225), and/or MH146 (SEQ ID NO. 232); a variable light chain region comprising ML01 (SEQ ID NO. 3), ML02 (SEQ ID NO. 4), ML05 (SEQ ID NO. 7), ML08 (SEQ ID NO. 10), ML10 (SEQ ID NO. 12), ML11 (SEQ ID NO. 13), ML12 (SEQ ID NO. 14), ML31 (SEQ ID NO. 31), ML32 (SEQ ID NO. 32), ML44 (SEQ ID NO. 42), ML49 (SEQ ID NO. 47), ML51 (SEQ ID NO. 48), ML52 (SEQ ID NO. 49), ML60 (SEQ ID NO. 56), ML71 (SEQ ID NO. 66), ML73 (SEQ ID NO. 68), ML74 (SEQ ID NO. 69), ML79 (SEQ ID NO. 74), ML84 (SEQ ID NO. 79), ML85 (SEQ ID NO. 80), ML92 (SEQ ID NO. 87), or ML94 (SEQ ID NO. 89); f) a combination CDRs, amino acid sequences, variable heavy chain regions, and/or variable light chain regions of a), b), c), d), or e) above; g) a combination of a LN02M variable light chain (“Light chain mutant”) and a LN02M variable heavy chain (“Heavy chain mutant”) described in Tables 4, 9, 10A through 10C, 11, 12A through 12D, 13A through 13D, or 14; h) a combination of a LN02M variable light chain comprising ML01 (SEQ ID NO. 3) with LN02M variable heavy chain comprising MH02 (SEQ ID NO. 96), MH04 (SEQ ID NO. 98), MH22 (SEQ ID NO. 116), MH23 (SEQ ID NO. 117), MH30 (SEQ ID NO. 124), MH31 (SEQ ID NO. 125), MH35 (SEQ ID NO. 129), MH36 (SEQ ID NO. 130), and/or MH37 (SEQ ID NO. 131); i) a combination of a LN02M variable light chain ML12 (SEQ ID NO. 14) with an LN02M variable heavy chain comprising MH02 (SEQ ID NO. 96), MH04 (SEQ ID NO. 98), MH22 (SEQ ID NO. 116), MH23 (SEQ ID NO. 117) MH30 (SEQ ID NO. 124), MH31 (SEQ ID NO. 125), MH35 (SEQ ID NO. 129), MH36 (SEQ ID NO. 130), and/or MH37 (SEQ ID NO. 131); j) a combination of a LN02M variable light chain ML23 (SEQ ID NO. 24) with LN02M variable heavy chain comprising MH31 (SEQ ID NO. 125), MH43 (SEQ ID NO. 136), MH48 (SEQ ID NO. 141), and MH51 (SEQ ID NO. 144); k) a combination of a LN02M variable light chain ML30 (SEQ ID NO. 30) with LN02M variable heavy chain comprising MH31 (SEQ ID NO. 125), MH43 (SEQ ID NO. 136), MH48 (SEQ ID NO. 141), or MH51 (SEQ ID NO. 144); I) a combination of a LN02M variable light chain ML31 (SEQ ID NO. 31) with LN02M variable heavy chain comprising MH02 (SEQ ID NO. 96), MH04 (SEQ ID NO. 98), MH22 (SEQ ID NO. 116), MH23 (SEQ ID NO. 117), MH30 (SEQ ID NO. 124), MH31 (SEQ ID NO. 125), MH35 (SEQ ID NO. 129), MH36 (SEQ ID NO. 130), MH37 (SEQ ID NO. 131), MH43 (SEQ ID NO. 136), MH48 (SEQ ID NO. 141), or MH51 (SEQ ID NO. 144); m) a combination of a LN02M variable light chain ML32 (SEQ ID NO. 32) with LN02M variable heavy chain MH31 (SEQ ID NO. 125); n) a combination of a LN02M variable light chain ML85 (SEQ ID NO. 80) with a LN02M variable heavy chain comprising MH31 (SEQ ID NO. 125), MH35 (SEQ ID NO. 129), MH43 (SEQ ID NO. 136), MH49 (SEQ ID NO. 142), MH60 (SEQ ID NO. 152), MH76 (SEQ ID NO. 166), MH111 (SEQ ID NO. 198), or MH112 (SEQ ID NO. 199); o) a combination of light and heavy chain substitutions shown in Table 9, optionally selected from the group consisting of the binding agents ML085 comprising the K93Y substitution on the light chain and wild-type LN02 heavy chain; Mx152 comprising the comprising the K93Y and E95Q substitutions to wild-type LN02 on the light chain and the S19H substitution to wild-type LN02 on the heavy chain; MX067 comprising the K93Y substitution to wild-type LN02 on the light chain and the S19H substitution to wild-type LN02 on the heavy chain; MX129 comprising the K93Y and T29S substitutions to wild-type LN02 on the light chain and the S19H substitution to wild-type LN02 on the heavy chain; MX130 comprising the K93Y and T29S substitutions to wild-type LN02 on the light chain and the T21Y substitution to wild-type LN02 heavy chain; ML126 comprising the K93Y and E95Q substitutions to wild-type LN02 on the light chain and the wild-type LN02 heavy chain; Mx175 comprising the K93Y and 197V substitutions to wild-type LN02 on the light chain and the S40A, Q42R, and T44G substitutions to wild-type LN02 on the heavy chain; Mx176 comprising the K93Y and 197V substitutions to wild-type LN02 on the light chain and the S40P, Q42R, G43K, and T44G substitutions to wild-type LN02 on the heavy chain; and, Mx181 comprising the K93Y and 197V substitutions to wild-type LN02 on the light chain and the S40P, Q42R, G43K, and T44G substitutions to wild-type LN02 on the heavy chain; and/or, p) a conservatively substituted variant of any of a) through o); and/or, q) a non-conservatively substituted variant comprising one or more amino acid substitutions outside of a CDR amino acid sequence of any of a) through p), or one to three substitutions within a CDR amino acid sequence of any of a) through p); a binding agent, preferably an antibody, including any of a) through p) above, exhibiting at least a 2-fold improvement in neutralization activity compared to LN02 and an equivalent or improved potency compared to ML085. In preferred embodiments, such a binding agent neutralizes human immunodeficiency virus (HIV) in an in vitro HIV neutralization assay and/or in vivo. In some embodiments, the binding agent exhibits neutralization of HIV-1 pseudoviruses BJOX (CRF07_BC), CE1176, TRO.11 (B), X1632 (G), CH119 (CRF07_BC), CNE55 (CRF01_AE), 25710 (C), CD0217(C) at a concentration is from 10²−10° ug/ml, or between 10⁰-10¹ μg/ml in an in vitro HIV neutralization assay. In some embodiments, the percent neutralization is at least about 50% or more. In some embodiments, the binding agent neutralizes a majority of the HIV-1 pseudoviruses tested at an IC₅₀ or IC₈₀ of less than 25 μg/ml. In some embodiments, the binding agent is an antibody, in preferred embodiments a monoclonal antibody, and even more preferred embodiments a human monoclonal antibody; or a derivative thereof. In some embodiments, the antibody isotype is IgG1 or IgG3. In some preferred embodiments, the binding agent comprises at least one heavy chain CDR amino acid sequence illustrated in FIG. 1 (i.e., corresponding to CDR1, CDR2, or CDR3 of LN02 bNab Heavy Chain chain underlined, and the substitutions thereto, shown therein) and/or described in any of SEQ ID NOS. 95-233; and/or a conservatively substituted variant thereof. In some preferred embodiments, the binding agent comprises at least one light chain CDR amino acid sequence in FIG. 1 (i.e., corresponding to CDR1, CDR2, or CDR3 of LN02 bNab Light chain underlined, and the substitutions thereto, shown therein) and/or described in any of SEQ ID NOS. 3-92; and/or a conservatively substituted variant thereof; and/or a non-conservatively substituted variant thereof as described above. In some preferred embodiments, the binding agent comprises at least one variable chain amino acid sequence selected from the group consisting of SEQ ID NOS. 3-92 and/or 95-233; and/or a conservatively substituted variant thereof; and/or a non-conservatively substituted variant thereof as described above. In some embodiments, the binding agent is derived from or based upon (e.g., includes the framework sequences of) a human antibody, human IgG, human IgG1, human IgG2, human IgG3, human IgG4, human IgM, human IgA, human IgA1, human IgA2, human IgD, human IgE, canine antibody, canine IgGA, canine IgGB, canine IgGC, canine IgGD, chicken antibody, chicken IgA, chicken IgD, chicken IgE, chicken IgG, chicken IgM, chicken IgY, goat antibody, goat IgG, mouse antibody, mouse IgG, pig antibody, rat antibody, or a camelid antibody. In some embodiments, this disclosure provides derivatives of such binding agents, such as one selected from the group consisting of an F_{ab}, F_{ab2}, Fab′ single chain antibody, Fᵥ, single chain, mono-specific antibody, bispecific antibody, trimeric antibody, multi-specific antibody, multivalent antibody, chimeric antibody, canine-human chimeric antibody, canine-mouse chimeric antibody, antibody comprising a canine Fc, humanized antibody, human antibody, caninized antibody, CDR-grafted antibody, shark antibody, nanobody, and camelid antibody. In some embodiments, the binding agent or derivative thereof comprises at least a least a first and second specificity, the first being against gp41 and the second being against a different antigen. In some embodiments, the binding agent or derivative thereof comprises one or more detectable labels fixably attached thereto (e.g., selected from the group consisting of fluorescein, DyLight, Cy3, Cy5, FITC, HiLyte Fluor 555, HiLyte Fluor 647, 5-carboxy-2,7-dichlorofluorescein, 5-carboxyfluorescein, 5-FAM, hydroxy tryptamine, 5-hydroxy tryptamine (5-HAT), 6-carboxyfluorescein (6-FAM), FITC, 6-carboxy-1,4-dichloro-2′,7′-dichlorofluorescein (TET), 6-carboxy-1,4-dichloro-2′,4′,5′,7′-tetrachlorofluorescein (HEX), 6-carboxy-4′,5′-dichloro-2′,7′-dimethoxyfluorescein (6-JOE), an Alexa fluor, Alexa fluor 350, Alexa fluor 405, Alexa fluor 430, Alexa fluor 488, Alexa fluor 500, Alexa fluor 514, Alexa fluor 532, Alexa fluor 546, Alexa fluor 555, Alexa fluor 568, Alexa fluor 594, Alexa fluor 610, Alexa fluor 633, Alexa fluor 635, Alexa fluor 647, Alexa fluor 660, Alexa fluor 680, Alexa fluor 700, Alexa fluor 750, a BODIPY fluorophores, BODIPY 492/515, BODIPY 493/503, BODIPY 500/510, BODIPY 505/515, BODIPY 530/550, BODIPY 542/563, BODIPY 558/568, BODIPY 564/570, BODIPY 576/589, BODIPY 581/591, BODIPY 630/650-X, BODIPY 650/665-X, BODIPY 665/676, FL, FL ATP, FI-Ceramide, R6G SE, TMR, TMR-X conjugate, TMR-X, SE, TR, TR ATP, TR-X SE, a rhodamine, rhodamine 110, rhodamine 123, rhodamine B, rhodamine B 200, rhodamine BB, rhodamine BG, rhodamine B extra, 5-carboxytetramethylrhodamine (5-TAMRA), 5 GLD, 6-carboxyrhodamine 6G, Lissamine, Lissamine Rhodamine B, Phallicidine, Phalloidine, rhodamine red, Rhod-2, 6-carboxy-X-rhodamine (ROX), carboxy-X-rhodamine (5-ROX), Sulphorhodamine B can C, Sulphorhodamine G Extra, 6-carboxytetramethylrhodamine (TAMRA), tetramethylrhodamine (TRITC), rhodamine WT, Texas Red, and Texas Red-X). In some embodiments, the binding agent or derivative thereof comprises one or more effector moieties fixably attached thereto (e.g., selected from the group consisting of a cytotoxic drug, toxin, diphtheria A chain, exotoxin A chain, ricin A chain, abrin A chain, curcin, crotin, phenomycin, enomycin, and radiochemical).

In some embodiments, this disclosure provides an isolated polynucleotide encoding any such binding agent(s), expression vectors comprising the same, and/or a host cell comprising the same. In some embodiments, this disclosure provides a composition comprising at least one such binding agent and/or or derivative thereof, at least one isolated polynucleotide encoding the same; at least one expression vector encoding the same, and/or, at least one host cell capable of producing the same (e.g., comprising at least one such polynucleotide and/or expression vector), and/or a combination thereof; and, a pharmaceutically acceptable carrier. In some embodiments, this disclosure also provides methods for manufacturing a binding agent and/or derivative thereof. In some embodiments, such methods for manufacturing comprising expressing one or more polynucleotides encoding a binding agent and/or derivative thereof of this disclosure in a host cell and purifying (e.g., to 90%, 95%, 99%, or 100% purity as may be determined by those of ordinary skill in the art using standard techniques) the same from the host cell, cell culture supernatant thereof, or the like, using standard techniques.

In some embodiments, this disclosure provides methods for detecting HIV on a cell, the method comprising contacting a test biological sample with a binding agent or derivative of this disclosure, and detecting the binding agent bound to the biological sample or components thereof. In some embodiments, such methods comprise comparing the amount of binding to the test biological sample or components thereof to the amount of binding to a control biological sample or components thereof, wherein increased binding to the test biological sample or components thereof relative to the control biological sample or components thereof indicates the presence of a cell expressing HIV in the test biological sample. In some embodiments, the test biological sample comprises, is, or is derived from mammalian blood or a component thereof. In some embodiments, the method is an in vivo method or the method is an in vitro method. In some embodiments, this disclosure provides methods for treating, preventing and/or ameliorating HIV infection and/or AIDS in a mammal comprising administering to the mammal at least one effective dose of a pharmaceutical composition comprising a binding agent and/or derivative thereof of this disclosure. In some embodiments, multiple doses of such a pharmaceutical composition are administered to the animal. In some embodiments, the binding agent and/or derivative thereof can be administered in a dosage amount of about 1 to 50 mg/kg. In some embodiments, this disclosure provides kits for detecting the expression of HIV in or on a cell, the kit comprising a binding agent and/or derivative thereof of this disclosure and optionally instructions for use. In some such embodiments, the binding agent, antibody, or derivative can be in lyophilized form.

The terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto.

As used herein, a subject or a host is meant to be an individual. The subject can include domesticated animals, such as cats and dogs, livestock (e.g., cattle, horses, pigs, sheep, and goats), laboratory animals (e.g., mice, rabbits, rats, guinea pigs) and birds. In one aspect, the subject is a mammal such as a primate or a human.

Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination).

Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed.

The term “combined” or “in combination” or “in conjunction” may refer to a physical combination of agents that are administered together or the use of two or more agents in a regimen (e.g., administered separately, physically and/or in time) for treating, preventing and/or ameliorating a particular disease.

When the terms treat, prevent, and/or ameliorate or derivatives thereof are used herein in connection with a given treatment for a given condition (e.g., preventing cancer infection by HIV), it is meant to convey that the treated patient either does not develop a clinically observable level of the condition at all, or develops it more slowly and/or to a lesser degree than he/she would have absent the treatment. These terms are not limited solely to a situation in which the patient experiences no aspect of the condition whatsoever. For example, a treatment will be said to have prevented the condition if it is given during exposure of a patient to a stimulus that would have been expected to produce a given manifestation of the condition, and results in the patient's experiencing fewer and/or milder symptoms of the condition than otherwise expected. For instance, a treatment can “prevent” infection by resulting in the patient's displaying only mild overt symptoms of the infection; it does not imply that there must have been no penetration of any cell by the infecting microorganism.

Similarly, reduce, reducing, and reduction as used herein in connection with prevention, treatment and/or amelioration of a given condition by a particular treatment typically refers to a subject developing an infection more slowly or to a lesser degree as compared to a control or basal level of developing an infection in the absence of a treatment (e.g., administration of one or more HIV binding agents). A reduction in the risk of infection may result in the patient's displaying only mild overt symptoms of the infection or delayed symptoms of infection; it does not imply that there must have been no penetration of any cell by the infecting microorganism.

All references cited within this disclosure are hereby incorporated by reference in their entirety. Certain embodiments are further described in the following examples. These embodiments are provided as examples only and are not intended to limit the scope of the claims in any way.

### EXAMPLES

### Example 1

### Lymph Node Donors

Selection of HIV-1 lymph node donors for the isolation of broadly neutralizing antibodies. As described in more detail elsewhere, in order to isolate the broadly neutralizing LN02 antibodies capable to broadly neutralize multi-Glade HIV-1 isolates in 107 plasma samples from chronically infected patients naïve to antiretroviral therapy were screened for the presence of high titers of antibodies able to neutralize a panel of nine (9) HIV-1 pseudoviruses from the Global Panel of HIV-1 reference strains (DeCamp, A. et al. Global panel of HIV-1 Env reference strains for standardized assessments of vaccine-elicited neutralizing antibodies. J Virol 88, 2489-2507 (2014)). This analysis resulted in the identification of eight (8) patients (FIG. 1) as lymph node donors for the subsequent isolation and characterization of potent broadly neutralizing antibodies. In particular, donor SA090 was identified as having high virus neutralizing activity (and for the lack of background activity against the negative control MLV pseudovirus). Germinal center and Memory IgG B cells from donor SA090 were sorted separately according to IgG (i.e. IgA and IgM negative cells), CD19 and CD38 expression (germinal center B cells are CD38 positive, which is not present on memory B cells) and interrogated for the production of HIV-1 neutralizing antibodies. In particular, highly pure IgG memory B cells and IgG germinal cells were seeded in separate plates as single cell micro-cultures on human feeder cells in the presence of Epstein-Barr Virus (EBV) (which also stimulate polyclonally memory B cells) and a cocktail composed TLR9 agonist CpG-2006, IL-2 (1000 IU/ml), IL-6 (10 ng/ml), IL-21 (10 ng/ml), and anti-BCR goat antibodies (BCR triggering). Supernatants from day 14 cultures were then tested in a primary screening using a 384-well based HIV-1 pseudoviruses neutralization assay (using in parallel two strains, CE1176 and BJOX2000, representative of Glade C and CRF07). Neutralization assays were undertaken on TZM-bl cells. In a 384-well plate, HIV-1 pseudoviruses that resulted in an output of 50-100×10⁴ relative light units (RLU) were incubated with B cell culture supernatants for 1 h at 37% (5% CO2) before the addition of 3000 TZM-bl cells. These were incubated for a further 72 h, after which supernatant was removed and 15 μl Steadylite reagent (Perkin Elmer) was added. Luciferase activity was detected 5 min later by reading the plates on a Synergy microplate luminometer (BioTek). The supernatants derived from germinal center B cells found to produce antibodies that cross-neutralize one or more of the HIV strains. The supernatants from these two cultures were further harvested and tested for their ability to neutralize pseudoviruses. One of these produced an antibody designated “LN02” and was found to neutralize HIV.

The LN02 antibody was characterized by determining the amino acid and nucleotide sequences of its variable regions and the complementarity determining regions (CDRs) ascertained. Accordingly, the binding agent termed “LN02” is an IgG1-type fully human monoclonal antibody having the CDR, VH and VL sequences shown in FIGS. 5 and 6 (e.g., SEQ ID NOS. 1, 93, 234 and 235). The LN02 antibody was also determined to be derived from the IGHV4-4*02 and IGLV3-21*01 germline genes and highly somatically mutated in variable genes of both heavy chain (31.2%) and kappa light chain (31.6%) compared to germ line. Recombinant LN02 antibody was produced and tested against the Global Panel of nine (9) HIV-1 reference pseudoviruses on TZM-bl cells, and found to be capable of neutralizing a majority of HIV-1 pseudoviruses.

Modified LN02 (LN02M) antibodies comprising modified CDR and non-CDR amino acid sequences were also produced using recombinant techniques. In order to identify LN02M broadly neutralizing antibodies with improved viral neutralization properties, a panel of single or multiple amino acid substitutions in LN02 were generated by site directed mutagenesis in expression vectors encoding the heavy or light chain sequences of the LN02 antibody. LN02M antibodies were produced by transient transfection of CHO cells with the wild type vector of the light chain co-transfected with one of the mutant vector for the heavy chain (Table 1, FIG. 6) or the wild type vector for the LN02 heavy chain co-transfected with one of the mutant light chain expression vectors (Table 2, FIG. 6). The transfected CHO cells were maintained in culture for 6 days, the medium was harvested and the mutant LN02 antibodies were purified from the cell culture supernatant with a protein A affinity column using standard protocols. The neutralization activity of the resulting LN02M antibodies was then evaluated in an antibody concentration response inhibition assay using the HIV-1 BaL virus in a TZM-bl luciferase reporter assay. In addition to neutralization activity, the protein production and concentration of mutant LN02 variants in Table 1 and 2 were evaluated to identify mutations that confer benefits in terms of improve antibody production and antibody stability that are needed for therapeutic antibodies. The amino acid sequences of exemplary LN02M variable regions produced and tested in this manner are illustrated in FIGS. 5, 6A through 6E, 7A through 7E, 8A through 8F, as well as SEQ ID NOS. 3-92, 95-233, 248-482, and 491-699.

Table 1 describes the neutralization activity of antibodies comprising the LN02M variable heavy chain amino acid sequences of SEQ ID NOS. 3-92 (identified in Table 1 and FIG. 6 as LN02 MH01 through MH147). Table 2 describes the neutralization activity of antibodies comprising the LN02M variable light chain amino acid sequences of SEQ ID NOS. 95-233 (identified in Table 2 and FIG. 6 as LN02 ML01 through ML94). Tables 1 and 2 compare the 1050 (mg/ml) against HIV pseudoviruses, and as a ratio to the neutralizing activity of LN02 (i.e., wild-type (WT) LN02 monoclonal antibody). The inhibitory concentration required for 50% neutralization (1050) of the BaL virus is indicated for the LN02 bNabs with heavy chain substitutions (Table 1) or light chain substitutions (Table 2) along with the ratio of wild type LN02 bNab 1050 tested in parallel divided by the mutant LN02 variant 1050. The latter value is used to limit inter-assay variability between viral neutralization assays performed on different days and to identify mutations that could confer a small but significant advantage in terms of neutralization activity to the LN02M relative to the wild type LN02 control. Additional neutralization data for LN02M antibodies are shown in FIGS. 9A through 9I as well as Tables 7A-7B, 8A through 8M, 10A through 10C, 11, 12A through 12D, 13A through 13D, and 14.

Surprisingly, several of the LN02M antibodies showed higher neutralizing activity than LN02; these include, for instance, the LN02M variable heavy chain regions MH01 (1.59), MH16 (1.69), MH22 (1.18), MH26 (1.40), MH30 (3.37), MH32 (1.32), MH35 (1.91), MH36 (1.37), MH37 (1.75), MH43 (1.90), MH44 (1.38), MH48 (2.12), MH49 (1.71), MH50 (2.74), MH51 (2.46), MH53 (1.45), MH59 (1.31), MH61 (1.43), MH64 (1.52), MH68 (1.12), MH73 (1.83), MH84 (1.16), MH89 (2.26), MH91 (1.36), MH92 (1.45), MH106 (1.16), MH107 (2.19), MH108 (1.91), MH111 (3.34), MH112 (2.77), MH115 (1.41), MH119 (1.32), MH120 (1.55), MH124 (1.67), MH 131 (1.55), MH135 (1.60), MH136 (1.84), MH138 (1.20), and MH146 (1.65); as well as the LN02M variable light chain regions ML01 (1.29), ML02 (1.93), ML05 (1.45), ML08 (2.31), ML10 (1.51), ML11 (1.25), ML12 (3.90), ML31 (5.74), ML32 (1.38), ML44 (1.57), ML49 (1.40), ML51 (1.10), ML52 (1.36), ML60 (1.17), ML71 (1.38), ML73 (1.20), ML74 (1.10), ML79 (1.46), ML84 (1.59), ML85 (9.94), and ML94 (6.42). Of note, LN02H antibodies comprising mutations LN02 MH30, LN02 MH111, LN02 ML12, LN02 ML31, LN02 ML85, LN02 ML92 and LN02 ML94 in Table 1 and 2 all demonstrate greater than 3-fold improved neutralization potency against the BaL virus relative the LN02 wild type control. FIG. 1 provides a summary of the amino acid substitutions in either the heavy or light chain of LN02 that confer an approximately >1.4-fold improved neutralization potency, a minimal effect (1.4- to 0.7-fold difference relative to LN02 WT) on neutralization activity or a <0.7-fold difference relative to wild type LN02 corresponding to mutations that induce a loss in neutralization activity.

### Example 2

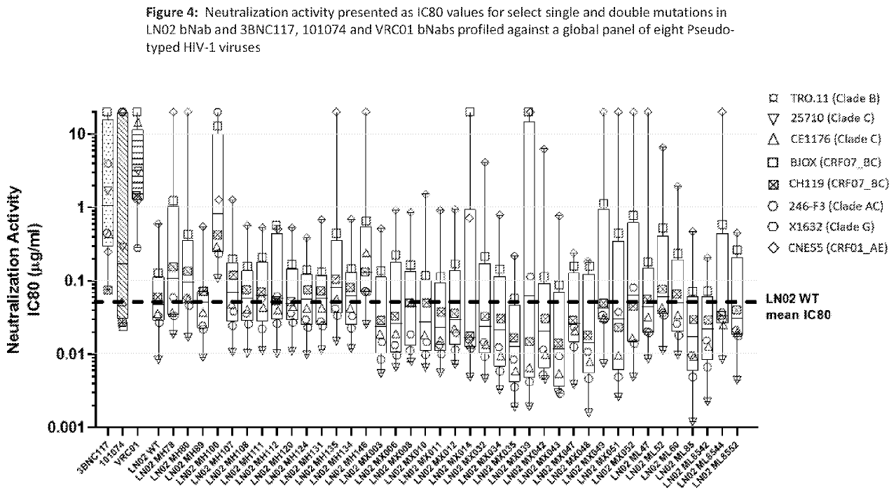

Neutralization of LN02 bNab and LN02 mutant variants against a global panel of eight pseudo-typed HIV-1 viral strains. A preliminary evaluation of the neutralization breadth of a select panel of LN02 bNabs variants with mutations in the heavy and/or light chain of LN02 was performed using a panel of eight pseudo-typed HIV-1 viruses. A summary of the 80% inhibitory concentration (IC₈₀) for each of the LN02 mutants (MH for heavy chain mutations, ML for light chain mutants and MX for mutations in both the heavy and light chain) with each of the eight pseudo-typed viruses (TRO.11, 25710, CD1176, BJOX, CH119, 246-F3, X1632, and CNE55) is shown in FIGS. 2-4. As a reference, FIG. 4 also shows the IC80 values for 3BNC117, 10-1074 and VRC01 against our global panel of pseudo-typed viruses. Representative concentration response viral neutralization curves are shown in FIG. 5 for LN02 mutants including LN02 ML85, LN02 ML8542 and LN02 MX48 that have significantly improved potency relative to the wild type LN02 and an overall improved neutralization profile compared to 10-1074 and 3BNC117 tested in parallel Table 3. Profiling of LN02M against the SVA-MLV pseudotyped virus control in FIG. 5 also demonstrates that the LN02M bNabs do not exhibit non-specific inhibition.

While certain embodiments have been described in terms of the preferred embodiments, it is understood that variations and modifications will occur to those skilled in the art. Therefore, it is intended that the appended claims cover all such equivalent variations that come within the scope of the following claims.

What is claimed is:

1. A binding agent comprising:
a) at least one CDR illustrated in FIG. 1, FIGS. 6A through 6E, FIGS. 7A-7E, or FIGS. 8A-F;
b) an amino acid sequence selected from the group consisting of SEQ ID NOS. 3-92 or 491-699;
c) an amino acid sequence selected from the group consisting of SEQ ID NOS. 95-233 or 248-482;
d) a variable heavy chain region comprising MH01 (SEQ ID NO. 95), MH16 (SEQ ID NO. 110), MH22 (SEQ ID NO. 116), MH26 (SEQ ID NO. 120), MH30 (SEQ ID NO. 124), MH32 (SEQ ID NO. 126), MH35 (SEQ ID NO. 129), MH36 (SEQ ID NO. 130), MH37 (SEQ ID NO. 131), MH43 (SEQ ID NO. 136), MH44 (SEQ ID NO. 137), MH48 (SEQ ID NO. 141), MH49 (SEQ ID NO. 142), MH50 (SEQ ID NO. 143), MH51 (SEQ ID NO. 144), MH53 (SEQ ID NO. 146), MH59 (SEQ ID NO. 151), MH61 (SEQ ID NO. 153), MH64 (SEQ ID NO. 156), MH68 (SEQ ID NO. 159), MH73 (SEQ ID NO. 163), MH84 (SEQ ID NO. 174), MH89 (SEQ ID NO. 177), MH91 (SEQ ID NO. 178), MH92 (SEQ ID NO. 179), MH106 (SEQ ID NO. 193), MH107 (SEQ ID NO. 194), MH108 (SEQ ID NO. 195), MH111 (SEQ ID NO. 198), MH112 (SEQ ID NO. 199), MH115 (SEQ ID NO. 202), MH119 (SEQ ID NO. 206), MH120 (SEQ ID NO. 207), MH124 (SEQ ID NO. 211), MH 131 (SEQ ID NO. 218), MH135 (SEQ ID NO. 222), MH136 (SEQ ID NO. 223), MH138 (SEQ ID NO. 225), and/or MH146 (SEQ ID NO. 232);
e) a variable light chain region comprising ML01 (SEQ ID NO. 3), ML02 (SEQ ID NO. 4), ML05 (SEQ ID NO. 7), ML08 (SEQ ID NO. 10), ML10 (SEQ ID NO. 12), ML11 (SEQ ID NO. 13), ML12 (SEQ ID NO. 14), ML31 (SEQ ID NO. 31), ML32 (SEQ ID NO. 32), ML44 (SEQ ID NO. 42), ML49 (SEQ ID NO. 47), ML51 (SEQ ID NO. 48), ML52 (SEQ ID NO. 49), ML60 (SEQ ID NO. 56), ML71 (SEQ ID NO. 66), ML73 (SEQ ID NO. 68), ML74 (SEQ ID NO. 69), ML79 (SEQ ID NO. 74), ML84 (SEQ ID NO. 79), ML85 (SEQ ID NO. 80), ML92 (SEQ ID NO. 87), or ML94 (SEQ ID NO. 89);
f) a combination CDRs, amino acid sequences, variable heavy chain regions, and/or variable light chain regions of a), b), c), d), or e);
g) a combination of a LN02M variable light chain and a LN02M variable heavy chain described in any of Tables 4, 9, 10A through 10C, 11, 12A through 12D, 13A through 13D, or 14;
h) a combination of a LN02M variable light chain comprising ML01 (SEQ ID NO. 3) with LN02M variable heavy chain comprising MH02 (SEQ ID NO. 96), MH04 (SEQ ID NO. 98), MH22 (SEQ ID NO. 116), MH23 (SEQ ID NO. 117), MH30 (SEQ ID NO. 124), MH31 (SEQ ID NO. 125), MH35 (SEQ ID NO. 129), MH36 (SEQ ID NO. 130), and/or MH37 (SEQ ID NO. 131);
i) a combination of a LN02M variable light chain ML12 (SEQ ID NO. 14) with an LN02M variable heavy chain comprising MH02 (SEQ ID NO. 96), MH04 (SEQ ID NO. 98), MH22 (SEQ ID NO. 116), MH23 (SEQ ID NO. 117) MH30 (SEQ ID NO. 124), MH31 (SEQ ID NO. 125), MH35 (SEQ ID NO. 129), MH36 (SEQ ID NO. 130), and/or MH37 (SEQ ID NO. 131);
j) a combination of a LN02M variable light chain ML23 (SEQ ID NO. 24) with LN02M variable heavy chain comprising MH31 (SEQ ID NO. 125), MH43 (SEQ ID NO. 136), MH48 (SEQ ID NO. 141), and MH51 (SEQ ID NO. 144);
k) a combination of a LN02M variable light chain ML30 (SEQ ID NO. 30) with LN02M variable heavy chain comprising MH31 (SEQ ID NO. 125), MH43 (SEQ ID NO. 136), MH48 (SEQ ID NO. 141), or MH51 (SEQ ID NO. 144);
l) a combination of a LN02M variable light chain ML31 (SEQ ID NO. 31) with LN02M variable heavy chain comprising MH02 (SEQ ID NO. 96), MH04 (SEQ ID NO. 98), MH22 (SEQ ID NO. 116), MH23 (SEQ ID NO. 117), MH30 (SEQ ID NO. 124), MH31 (SEQ ID NO. 125), MH35 (SEQ ID NO. 129), MH36 (SEQ ID NO. 130), MH37 (SEQ ID NO. 131), MH43 (SEQ ID NO. 136), MH48 (SEQ ID NO. 141), or MH51 (SEQ ID NO. 144);
m) a combination of a LN02M variable light chain ML32 (SEQ ID NO. 32) with LN02M variable heavy chain MH31 (SEQ ID NO. 125);
n) a combination of a LN02M variable light chain ML85 (SEQ ID NO. 80) with a LN02M variable heavy chain comprising MH31 (SEQ ID NO. 125), MH35 (SEQ ID NO. 129), MH43 (SEQ ID NO. 136), MH49 (SEQ ID NO. 142), MH60 (SEQ ID NO. 152), MH76 (SEQ ID NO. 166), MH111 (SEQ ID NO. 198), or MH112 (SEQ ID NO. 199);
o) a combination of light and heavy chain substitutions shown in Table 9, optionally selected from the group consisting of the binding agents ML085 comprising the K93Y substitution on the light chain and wild-type LN02 heavy chain; Mx152 comprising the comprising the K93Y and E95Q substitutions to wild-type LN02 on the light chain and the S19H substitution to wild-type LN02 on the heavy chain; MX067 comprising the K93Y substitution to wild-type LN02 on the light chain and the S19H substitution to wild-type LN02 on the heavy chain; MX129 comprising the K93Y and T29S substitutions to wild-type LN02 on the light chain and the S19H substitution to wild-type LN02 on the heavy chain; MX130 comprising the K93Y and T29S substitutions to wild-type LN02 on the light chain and the T21Y substitution to wild-type LN02 heavy chain; ML126 comprising the K93Y and E95Q substitutions to wild-type LN02 on the light chain and the wild-type LN02 heavy chain; Mx175 comprising the K93Y and 197V substitutions to wild-type LN02 on the light chain and the S40A, Q42R, and T44G substitutions to wild-type LN02 on the heavy chain; Mx176 comprising the K93Y and 197V substitutions to wild-type LN02 on the light chain and the S40P, Q42R, G43K, and T44G substitutions to wild-type LN02 on the heavy chain; and, Mx181 comprising the K93Y and 197V substitutions to wild-type LN02 on the light chain and the S40P, Q42R, G43K, and T44G substitutions to wild-type LN02 on the heavy chain; and,
p) and conservatively substituted variant of a) through o); or,
a binding agent, preferably an antibody, including any of a) through p) above, exhibiting at least a 2-fold improvement in neutralization activity compared to LN02 and an equivalent or improved potency compared to ML085.

2. The binding agent of claim 1 wherein the binding agent neutralizes human immunodeficiency virus (HIV) in an in vitro HIV neutralization assay and/or in vivo.

3. The binding agent of claim 1 or 2 wherein the binding agent exhibits neutralization of HIV-1 pseudoviruses BJOX (CRF07_BC), CE1176, TRO.11 (B), X1632 (G), CH119 (CRF07_BC), CNE55 (CRF01_AE), 25710 (C), CD0217(C) at a concentration is from 10²−10° ug/ml, or between 10⁰-10¹ μg/ml.

4. The binding agent of claim 2 or 3 wherein the percent neutralization is at least about 50%.

5. The binding agent of any one of claims 1-4 wherein the binding agent neutralizes a majority of the HIV-1 pseudoviruses tested at an IC₅₀ or IC₈₀ of less than 25 μg/ml.

6. The binding agent of any one of claims 1-5 that is an antibody.

7. The binding agent of claim 6 that is an isolated monoclonal antibody.

8. The binding agent of claim 6 wherein the monoclonal antibody is a human monoclonal antibody.

9. The binding agent of claim 7 or 8 wherein the antibody isotype is IgG1 or IgG3.

10. The binding agent of claim 1 comprising at least one heavy chain CDR amino acid sequence illustrated in FIG. 1 and/or described in any of SEQ ID NOS. 95-233; and/or a conservatively substituted variant thereof.

11. The binding agent of claim 1 comprising at least one light chain CDR amino acid sequence in FIG. 1 and/or described in any of SEQ ID NOS. 3-92; and/or a conservatively substituted variant thereof.

12. The binding agent of claim 1 comprising at least one variable chain amino acid sequence selected from the group consisting of SEQ ID NOS. 3-92 and/or 95-233; and/or a conservatively substituted variant thereof.

13. The binding agent of any one of claims 1-12 derived from a human antibody, human IgG, human IgG1, human IgG2, human IgG3, human IgG4, human IgM, human IgA, human IgA1, human IgA2, human IgD, human IgE, canine antibody, canine IgGA, canine IgGB, canine IgGC, canine IgGD, chicken antibody, chicken IgA, chicken IgD, chicken IgE, chicken IgG, chicken IgM, chicken IgY, goat antibody, goat IgG, mouse antibody, mouse IgG, pig antibody, and rat antibody.

14. A derivative of a binding agent of any one of claims 1-13.

15. The derivative of claim 14 selected from the group consisting of an F_{ab}, F_{ab2}, Fab′ single chain antibody, Fᵥ, single chain, mono-specific antibody, bispecific antibody, trimeric antibody, multi-specific antibody, multivalent antibody, chimeric antibody, canine-human chimeric antibody, canine-mouse chimeric antibody, antibody comprising a canine Fc, humanized antibody, human antibody, caninized antibody, CDR-grafted antibody, shark antibody, nanobody, and camelid antibody.

16. The binding agent or derivative of any one of claims 1-15 comprising at least a least a first and second specificity, the first being against gp41 and the second being against a different antigen.

17. The binding agent or derivative of any one of claims 1-16 comprising a detectable label fixably attached thereto.

18. The binding agent of claim 17 wherein the detectable label is selected from the group consisting of fluorescein, DyLight, Cy3, Cy5, FITC, HiLyte Fluor 555, HiLyte Fluor 647, 5-carboxy-2,7-dichlorofluorescein, 5-carboxyfluorescein, 5-FAM, hydroxy tryptamine, 5-hydroxy tryptamine (5-HAT), 6-carboxyfluorescein (6-FAM), FITC, 6-carboxy-1,4-dichloro-2′,7′-dichlorofluorescein (TET), 6-carboxy-1,4-dichloro-2′,4′,5′,7′-tetra-chlorofluorescein (HEX), 6-carboxy-4′,5′-dichloro-2′,7′-dimethoxyfluorescein (6-JOE), an Alexa fluor, Alexa fluor 350, Alexa fluor 405, Alexa fluor 430, Alexa fluor 488, Alexa fluor 500, Alexa fluor 514, Alexa fluor 532, Alexa fluor 546, Alexa fluor 555, Alexa fluor 568, Alexa fluor 594, Alexa fluor 610, Alexa fluor 633, Alexa fluor 635, Alexa fluor 647, Alexa fluor 660, Alexa fluor 680, Alexa fluor 700, Alexa fluor 750, a BODIPY fluorophores, BODIPY 492/515, BODIPY 493/503, BODIPY 500/510, BODIPY 505/515, BODIPY 530/550, BODIPY 542/563, BODIPY 558/568, BODIPY 564/570, BODIPY 576/589, BODIPY 581/591, BODIPY 630/650-X, BODIPY 650/665-X, BODIPY 665/676, FL, FL ATP, FI-Ceramide, R6G SE, TMR, TMR-X conjugate, TMR-X, SE, TR, TR ATP, TR-X SE, a rhodamine, rhodamine 110, rhodamine 123, rhodamine B, rhodamine B 200, rhodamine BB, rhodamine BG, rhodamine B extra, 5-carboxytetramethylrhodamine (5-TAMRA), 5 GLD, 6-carboxyrhodamine 6G, Lissamine, Lissamine Rhodamine B, Phallicidine, Phalloidine, rhodamine red, Rhod-2, 6-carboxy-X-rhodamine (ROX), carboxy-X-rhodamine (5-ROX), Sulphorhodamine B can C, Sulphorhodamine G Extra, 6-carboxytetramethylrhodamine (TAMRA), tetramethylrhodamine (TRITC), rhodamine WT, Texas Red, and Texas Red-X.

19. The binding agent or derivative of any one of claims 1-18 or derivative thereof comprising an effector moiety fixably attached thereto.

20. The binding agent or derivative of claim 19 wherein the effector moiety is selected from the group consisting of a cytotoxic drug, toxin, diphtheria A chain, exotoxin A chain, ricin A chain, abrin A chain, curcin, crotin, phenomycin, enomycin, and radiochemical.

21. An isolated polynucleotide encoding a binding agent of any one of claims 1-15.

22. An expression vector comprising one or more polynucleotides of claim 21.

23. A host cell comprising the isolated polynucleotide of claim 21 and/or the expression vector of claim 22.

24. A composition comprising at least one binding agent or derivative of any one of claims 1-20; at least one isolated polynucleotide of claim 21; or at least one expression vector of claim 23; and/or, at least one host cell of claim 23; or a combination thereof; and, a pharmaceutically acceptable carrier.

25. A method for detecting HIV on a cell, the method comprising contacting a test biological sample with a binding agent or derivative of any one of claims 1-20 and detecting the binding agent bound to the biological sample or components thereof.

26. The method of claim 25, further comprising comparing the amount of binding to the test biological sample or components thereof to the amount of binding to a control biological sample or components thereof, wherein increased binding to the test biological sample or components thereof relative to the control biological sample or components thereof indicates the presence of a cell expressing HIV in the test biological sample.

27. The method of claim 25 or 26 wherein the test biological sample is mammalian blood.

28. The method of any one of claims 25-27 wherein the method is an in vivo method.

29. The method of any one of claims 25-27 wherein the method is an in vitro method.

30. A method for treating, preventing and/or ameliorating HIV infection and/or AIDS in a mammal comprising administering to the mammal at least one effective dose of a pharmaceutical composition comprising a binding agent or derivative of any one of claims 1-20.

31. The method of claim 30 wherein multiple doses are administered to the animal.

32. The method of claim 30 or 31 wherein the binding agent is administered in a dosage amount of about 1 to 50 mg/kg.

33. A kit for detecting the expression of HIV in or on a cell, the kit comprising a binding agent or derivative of any one of claims 1-20 and instructions for use.

34. The kit of claim 33 wherein the binding agent, antibody, or derivative is in lyophilized form.
